SEQUENCE LISTING (810 sequences)

SEQ ID NO: 1 - DNA, 20 bp, synthetic construct
  source 1..20: mol_type = other DNA
cacacctgatcaaatttctc

SEQ ID NO: 2 - DNA, 20 bp, synthetic construct
  source 1..20: mol_type = other DNA
cgaggttttgttccgaagcc

SEQ ID NO: 3 - DNA, 20 bp, synthetic construct
  source 1..20: mol_type = other DNA
ggggtaatgtgacgaggttt

SEQ ID NO: 4 - DNA, 20 bp, synthetic construct
  source 1..20: mol_type = other DNA
ttaatattcgcgcggcttcg

SEQ ID NO: 5 - DNA, 20 bp, synthetic construct
  source 1..20: mol_type = other DNA
tttcctttaatattcgcgcg

SEQ ID NO: 6 - DNA, 20 bp, synthetic construct
  source 1..20: mol_type = other DNA
gtttcctttaatattcgcgc

SEQ ID NO: 7 - DNA, 20 bp, synthetic construct
  source 1..20: mol_type = other DNA
ttcggctcacgctgggttgt

SEQ ID NO: 8 - DNA, 20 bp, synthetic construct
  source 1..20: mol_type = other DNA
gggtaatgtgacgaggtttt

SEQ ID NO: 9 - DNA, 20 bp, synthetic construct
  source 1..20: mol_type = other DNA
gcacacctgatcaaatttct

SEQ ID NO: 10 - DNA, 20 bp, synthetic construct
  source 1..20: mol_type = other DNA
ctgctcagtcttcagaaaat

SEQ ID NO: 11 - DNA, 20 bp, synthetic construct
  source 1..20: mol_type = other DNA
agtttaagagggtctgtgtg

SEQ ID NO: 12 - DNA, 20 bp, synthetic construct
  source 1..20: mol_type = other DNA
aagtttaagagggtctgtgt

SEQ ID NO: 13 - DNA, 20 bp, synthetic construct
  source 1..20: mol_type = other DNA
tgttggcagaggatggtcaa

SEQ ID NO: 14 - DNA, 20 bp, synthetic construct
  source 1..20: mol_type = other DNA
actgttggcagaggatggtc

SEQ ID NO: 15 - DNA, 20 bp, synthetic construct
  source 1..20: mol_type = other DNA
ggttctgatgtttgtagtgt

SEQ ID NO: 16 - DNA, 20 bp, synthetic construct
  source 1..20: mol_type = other DNA
tggttctgatgtttgtagtg

SEQ ID NO: 17 - DNA, 20 bp, synthetic construct
  source 1..20: mol_type = other DNA
gggcttggttctgatgtttg

SEQ ID NO: 18 - DNA, 20 bp, synthetic construct
  source 1..20: mol_type = other DNA
cgggcttggttctgatgttt

SEQ ID NO: 19 - DNA, 20 bp, synthetic construct
  source 1..20: mol_type = other DNA
tggggtcatatgttcctggc

SEQ ID NO: 20 - DNA, 20 bp, synthetic construct
  source 1..20: mol_type = other DNA
ttggggtcatatgttcctgg

SEQ ID NO: 21 - DNA, 20 bp, synthetic construct
  source 1..20: mol_type = other DNA
aatattcgcgcggcttcggc

SEQ ID NO: 22 - DNA, 20 bp, synthetic construct
  source 1..20: mol_type = other DNA
taatattcgcgcggcttcgg

SEQ ID NO: 23 - DNA, 20 bp, synthetic construct
  source 1..20: mol_type = other DNA
ttaatattcgcgcggcttcg

SEQ ID NO: 24 - DNA, 20 bp, synthetic construct
  source 1..20: mol_type = other DNA
tttaatattcgcgcggcttc

SEQ ID NO: 25 - DNA, 20 bp, synthetic construct
  source 1..20: mol_type = other DNA
ctttaatattcgcgcggctt

SEQ ID NO: 26 - DNA, 20 bp, synthetic construct
  source 1..20: mol_type = other DNA
tcctttaatattcgcgcggc

SEQ ID NO: 27 - DNA, 20 bp, synthetic construct
  source 1..20: mol_type = other DNA
ttcctttaatattcgcgcgg

SEQ ID NO: 28 - DNA, 20 bp, synthetic construct
  source 1..20: mol_type = other DNA
tttcctttaatattcgcgcg

SEQ ID NO: 29 - DNA, 20 bp, synthetic construct
  source 1..20: mol_type = other DNA
gtttcctttaatattcgcgc

SEQ ID NO: 30 - DNA, 20 bp, synthetic construct
  source 1..20: mol_type = other DNA
ggttccaagcatcttgccct

SEQ ID NO: 31 - DNA, 20 bp, synthetic construct
  source 1..20: mol_type = other DNA
cggttccaagcatcttgccc

SEQ ID NO: 32 - DNA, 20 bp, synthetic construct
  source 1..20: mol_type = other DNA
ccggttccaagcatcttgcc

SEQ ID NO: 33 - DNA, 20 bp, synthetic construct
  source 1..20: mol_type = other DNA
tccggttccaagcatcttgc

SEQ ID NO: 34 - DNA, 20 bp, synthetic construct
  source 1..20: mol_type = other DNA
gtccggttccaagcatcttg

SEQ ID NO: 35 - DNA, 20 bp, synthetic construct
  source 1..20: mol_type = other DNA
ggtccggttccaagcatctt

SEQ ID NO: 36 - DNA, 20 bp, synthetic construct
  source 1..20: mol_type = other DNA
aggtccggttccaagcatct

SEQ ID NO: 37 - DNA, 20 bp, synthetic construct
  source 1..20: mol_type = other DNA
caggtccggttccaagcatc

SEQ ID NO: 38 - DNA, 20 bp, synthetic construct
  source 1..20: mol_type = other DNA
gcaggtccggttccaagcat

SEQ ID NO: 39 - DNA, 20 bp, synthetic construct
  source 1..20: mol_type = other DNA
tcattccggctctttaggcc

SEQ ID NO: 40 - DNA, 20 bp, synthetic construct
  source 1..20: mol_type = other DNA
ctcattccggctctttaggc

SEQ ID NO: 41 - DNA, 20 bp, synthetic construct
  source 1..20: mol_type = other DNA
cctcattccggctctttagg

SEQ ID NO: 42 - DNA, 20 bp, synthetic construct
  source 1..20: mol_type = other DNA
tcctcattccggctctttag

SEQ ID NO: 43 - DNA, 20 bp, synthetic construct
  source 1..20: mol_type = other DNA
ttcctcattccggctcttta

SEQ ID NO: 44 - DNA, 20 bp, synthetic construct
  source 1..20: mol_type = other DNA
tttcctcattccggctcttt

SEQ ID NO: 45 - DNA, 20 bp, synthetic construct
  source 1..20: mol_type = other DNA
gtttcctcattccggctctt

SEQ ID NO: 46 - DNA, 20 bp, synthetic construct
  source 1..20: mol_type = other DNA
ggtttcctcattccggctct

SEQ ID NO: 47 - DNA, 20 bp, synthetic construct
  source 1..20: mol_type = other DNA
tggtttcctcattccggctc

SEQ ID NO: 48 - DNA, 20 bp, synthetic construct
  source 1..20: mol_type = other DNA
ctggtttcctcattccggct

SEQ ID NO: 49 - DNA, 20 bp, synthetic construct
  source 1..20: mol_type = other DNA
cctggtttcctcattccggc

SEQ ID NO: 50 - DNA, 20 bp, synthetic construct
  source 1..20: mol_type = other DNA
ccctggtttcctcattccgg

SEQ ID NO: 51 - DNA, 20 bp, synthetic construct
  source 1..20: mol_type = other DNA
gccctggtttcctcattccg

SEQ ID NO: 52 - DNA, 20 bp, synthetic construct
  source 1..20: mol_type = other DNA
ggccctggtttcctcattcc

SEQ ID NO: 53 - DNA, 20 bp, synthetic construct
  source 1..20: mol_type = other DNA
ctttcctctcattggcatct

SEQ ID NO: 54 - DNA, 20 bp, synthetic construct
  source 1..20: mol_type = other DNA
cctttcctctcattggcatc

SEQ ID NO: 55 - DNA, 20 bp, synthetic construct
  source 1..20: mol_type = other DNA
acctttcctctcattggcat

SEQ ID NO: 56 - DNA, 20 bp, synthetic construct
  source 1..20: mol_type = other DNA
cacgctgggttgtgagaatc

SEQ ID NO: 57 - DNA, 20 bp, synthetic construct
  source 1..20: mol_type = other DNA
tcacgctgggttgtgagaat

SEQ ID NO: 58 - DNA, 20 bp, synthetic construct
  source 1..20: mol_type = other DNA
ctcacgctgggttgtgagaa

SEQ ID NO: 59 - DNA, 20 bp, synthetic construct
  source 1..20: mol_type = other DNA
gctcacgctgggttgtgaga

SEQ ID NO: 60 - DNA, 20 bp, synthetic construct
  source 1..20: mol_type = other DNA
ttcggctcacgctgggttgt

SEQ ID NO: 61 - DNA, 20 bp, synthetic construct
  source 1..20: mol_type = other DNA
gattcatgcccttctctttg

SEQ ID NO: 62 - DNA, 20 bp, synthetic construct
  source 1..20: mol_type = other DNA
cgattcatgcccttctcttt

SEQ ID NO: 63 - DNA, 20 bp, synthetic construct
  source 1..20: mol_type = other DNA
ccgattcatgcccttctctt

SEQ ID NO: 64 - DNA, 20 bp, synthetic construct
  source 1..20: mol_type = other DNA
cccgattcatgcccttctct

SEQ ID NO: 65 - DNA, 20 bp, synthetic construct
  source 1..20: mol_type = other DNA
tcccgattcatgcccttctc

SEQ ID NO: 66 - DNA, 20 bp, synthetic construct
  source 1..20: mol_type = other DNA
atcccgattcatgcccttct

SEQ ID NO: 67 - DNA, 20 bp, synthetic construct
  source 1..20: mol_type = other DNA
catcccgattcatgcccttc

SEQ ID NO: 68 - DNA, 20 bp, synthetic construct
  source 1..20: mol_type = other DNA
tcatcccgattcatgccctt

SEQ ID NO: 69 - DNA, 20 bp, synthetic construct
  source 1..20: mol_type = other DNA
atcatcccgattcatgccct

SEQ ID NO: 70 - DNA, 20 bp, synthetic construct
  source 1..20: mol_type = other DNA
gatcatcccgattcatgccc

SEQ ID NO: 71 - DNA, 20 bp, synthetic construct
  source 1..20: mol_type = other DNA
cgatcatcccgattcatgcc

SEQ ID NO: 72 - DNA, 20 bp, synthetic construct
  source 1..20: mol_type = other DNA
ccatggatccgatcatcccg

SEQ ID NO: 73 - DNA, 20 bp, synthetic construct
  source 1..20: mol_type = other DNA
tccatggatccgatcatccc

SEQ ID NO: 74 - DNA, 20 bp, synthetic construct
  source 1..20: mol_type = other DNA
gtcgtgtaccagctgctgct

SEQ ID NO: 75 - DNA, 20 bp, synthetic construct
  source 1..20: mol_type = other DNA
tgtcgtgtaccagctgctgc

SEQ ID NO: 76 - DNA, 20 bp, synthetic construct
  source 1..20: mol_type = other DNA
ttgtcgtgtaccagctgctg

SEQ ID NO: 77 - DNA, 20 bp, synthetic construct
  source 1..20: mol_type = other DNA
cttgtcgtgtaccagctgct

SEQ ID NO: 78 - DNA, 20 bp, synthetic construct
  source 1..20: mol_type = other DNA
acttgtcgtgtaccagctgc

SEQ ID NO: 79 - DNA, 20 bp, synthetic construct
  source 1..20: mol_type = other DNA
gcagtacttgtcgtgtacca

SEQ ID NO: 80 - DNA, 20 bp, synthetic construct
  source 1..20: mol_type = other DNA
tgcagtacttgtcgtgtacc

SEQ ID NO: 81 - DNA, 20 bp, synthetic construct
  source 1..20: mol_type = other DNA
ttgcagtacttgtcgtgtac

SEQ ID NO: 82 - DNA, 20 bp, synthetic construct
  source 1..20: mol_type = other DNA
tttgcagtacttgtcgtgta

SEQ ID NO: 83 - DNA, 20 bp, synthetic construct
  source 1..20: mol_type = other DNA
ctttgcagtacttgtcgtgt

SEQ ID NO: 84 - DNA, 20 bp, synthetic construct
  source 1..20: mol_type = other DNA
tctttgcagtacttgtcgtg

SEQ ID NO: 85 - DNA, 20 bp, synthetic construct
  source 1..20: mol_type = other DNA
atctttgcagtacttgtcgt

SEQ ID NO: 86 - DNA, 20 bp, synthetic construct
  source 1..20: mol_type = other DNA
gatctttgcagtacttgtcg

SEQ ID NO: 87 - DNA, 20 bp, synthetic construct
  source 1..20: mol_type = other DNA
ggttttgttccgaagcccat

SEQ ID NO: 88 - DNA, 20 bp, synthetic construct
  source 1..20: mol_type = other DNA
aggttttgttccgaagccca

SEQ ID NO: 89 - DNA, 20 bp, synthetic construct
  source 1..20: mol_type = other DNA
gaggttttgttccgaagccc

SEQ ID NO: 90 - DNA, 20 bp, synthetic construct
  source 1..20: mol_type = other DNA
cgaggttttgttccgaagcc

SEQ ID NO: 91 - DNA, 20 bp, synthetic construct
  source 1..20: mol_type = other DNA
acgaggttttgttccgaagc

SEQ ID NO: 92 - DNA, 20 bp, synthetic construct
  source 1..20: mol_type = other DNA
gacgaggttttgttccgaag

SEQ ID NO: 93 - DNA, 20 bp, synthetic construct
  source 1..20: mol_type = other DNA
tgacgaggttttgttccgaa

SEQ ID NO: 94 - DNA, 20 bp, synthetic construct
  source 1..20: mol_type = other DNA
gtgacgaggttttgttccga

SEQ ID NO: 95 - DNA, 20 bp, synthetic construct
  source 1..20: mol_type = other DNA
tgtgacgaggttttgttccg

SEQ ID NO: 96 - DNA, 20 bp, synthetic construct
  source 1..20: mol_type = other DNA
gtaatgtgacgaggttttgt

SEQ ID NO: 97 - DNA, 20 bp, synthetic construct
  source 1..20: mol_type = other DNA
ggtaatgtgacgaggttttg

SEQ ID NO: 98 - DNA, 20 bp, synthetic construct
  source 1..20: mol_type = other DNA
gggtaatgtgacgaggtttt

SEQ ID NO: 99 - DNA, 20 bp, synthetic construct
  source 1..20: mol_type = other DNA
ggggtaatgtgacgaggttt

SEQ ID NO: 100 - DNA, 20 bp, synthetic construct
  source 1..20: mol_type = other DNA
gggggtaatgtgacgaggtt

SEQ ID NO: 101 - DNA, 20 bp, synthetic construct
  source 1..20: mol_type = other DNA
agggggtaatgtgacgaggt

SEQ ID NO: 102 - DNA, 20 bp, synthetic construct
  source 1..20: mol_type = other DNA
aagggggtaatgtgacgagg

SEQ ID NO: 103 - DNA, 20 bp, synthetic construct
  source 1..20: mol_type = other DNA
gaagggggtaatgtgacgag

SEQ ID NO: 104 - DNA, 20 bp, synthetic construct
  source 1..20: mol_type = other DNA
tgaagggggtaatgtgacga

SEQ ID NO: 105 - DNA, 20 bp, synthetic construct
  source 1..20: mol_type = other DNA
aggtcccaaatcccatgagg

SEQ ID NO: 106 - DNA, 20 bp, synthetic construct
  source 1..20: mol_type = other DNA
gaggtcccaaatcccatgag

SEQ ID NO: 107 - DNA, 20 bp, synthetic construct
  source 1..20: mol_type = other DNA
ggaggtcccaaatcccatga

SEQ ID NO: 108 - DNA, 20 bp, synthetic construct
  source 1..20: mol_type = other DNA
cacctgatcaaatttctcct

SEQ ID NO: 109 - DNA, 20 bp, synthetic construct
  source 1..20: mol_type = other DNA
acacctgatcaaatttctcc

SEQ ID NO: 110 - DNA, 20 bp, synthetic construct
  source 1..20: mol_type = other DNA
cacacctgatcaaatttctc

SEQ ID NO: 111 - DNA, 20 bp, synthetic construct
  source 1..20: mol_type = other DNA
gcacacctgatcaaatttct

SEQ ID NO: 112 - DNA, 20 bp, synthetic construct
  source 1..20: mol_type = other DNA
ggcacacctgatcaaatttc

SEQ ID NO: 113 - DNA, 20 bp, synthetic construct
  source 1..20: mol_type = other DNA
tggcacacctgatcaaattt

SEQ ID NO: 114 - DNA, 20 bp, synthetic construct
  source 1..20: mol_type = other DNA
ctggcacacctgatcaaatt

SEQ ID NO: 115 - DNA, 20 bp, synthetic construct
  source 1..20: mol_type = other DNA
actggcacacctgatcaaat

SEQ ID NO: 116 - DNA, 20 bp, synthetic construct
  source 1..20: mol_type = other DNA
cactggcacacctgatcaaa

SEQ ID NO: 117 - DNA, 20 bp, synthetic construct
  source 1..20: mol_type = other DNA
ccactggcacacctgatcaa

SEQ ID NO: 118 - DNA, 20 bp, synthetic construct
  source 1..20: mol_type = other DNA
cccactggcacacctgatca

SEQ ID NO: 119 - DNA, 20 bp, synthetic construct
  source 1..20: mol_type = other DNA
tttggatcagcgagttcttg

SEQ ID NO: 120 - DNA, 20 bp, synthetic construct
  source 1..20: mol_type = other DNA
atttggatcagcgagttctt

SEQ ID NO: 121 - DNA, 20 bp, synthetic construct
  source 1..20: mol_type = other DNA
catttggatcagcgagttct

SEQ ID NO: 122 - DNA, 20 bp, synthetic construct
  source 1..20: mol_type = other DNA
ttaggacatggttcatggta

SEQ ID NO: 123 - DNA, 20 bp, synthetic construct
  source 1..20: mol_type = other DNA
cttaggacatggttcatggt

SEQ ID NO: 124 - DNA, 20 bp, synthetic construct
  source 1..20: mol_type = other DNA
gcttaggacatggttcatgg

SEQ ID NO: 125 - DNA, 20 bp, synthetic construct
  source 1..20: mol_type = other DNA
agcttaggacatggttcatg

SEQ ID NO: 126 - DNA, 20 bp, synthetic construct
  source 1..20: mol_type = other DNA
cagcttaggacatggttcat

SEQ ID NO: 127 - DNA, 20 bp, synthetic construct
  source 1..20: mol_type = other DNA
acagcttaggacatggttca

SEQ ID NO: 128 - DNA, 20 bp, synthetic construct
  source 1..20: mol_type = other DNA
cacagcttaggacatggttc

SEQ ID NO: 129 - DNA, 20 bp, synthetic construct
  source 1..20: mol_type = other DNA
acacagcttaggacatggtt

SEQ ID NO: 130 - DNA, 20 bp, synthetic construct
  source 1..20: mol_type = other DNA
gacacagcttaggacatggt

SEQ ID NO: 131 - DNA, 20 bp, synthetic construct
  source 1..20: mol_type = other DNA
tgacacagcttaggacatgg

SEQ ID NO: 132 - DNA, 20 bp, synthetic construct
  source 1..20: mol_type = other DNA
ttgacacagcttaggacatg

SEQ ID NO: 133 - DNA, 20 bp, synthetic construct
  source 1..20: mol_type = other DNA
cttgacacagcttaggacat

SEQ ID NO: 134 - DNA, 20 bp, synthetic construct
  source 1..20: mol_type = other DNA
atgggcgaagtcctttgggg

SEQ ID NO: 135 - DNA, 20 bp, synthetic construct
  source 1..20: mol_type = other DNA
tatgggcgaagtcctttggg

SEQ ID NO: 136 - DNA, 20 bp, synthetic construct
  source 1..20: mol_type = other DNA
ttatgggcgaagtcctttgg

SEQ ID NO: 137 - DNA, 20 bp, synthetic construct
  source 1..20: mol_type = other DNA
tccttgatatcctgctggat

SEQ ID NO: 138 - DNA, 20 bp, synthetic construct
  source 1..20: mol_type = other DNA
ctccttgatatcctgctgga

SEQ ID NO: 139 - DNA, 20 bp, synthetic construct
  source 1..20: mol_type = other DNA
gctccttgatatcctgctgg

SEQ ID NO: 140 - DNA, 20 bp, synthetic construct
  source 1..20: mol_type = other DNA
agctccttgatatcctgctg

SEQ ID NO: 141 - DNA, 20 bp, synthetic construct
  source 1..20: mol_type = other DNA
cagctccttgatatcctgct

SEQ ID NO: 142 - DNA, 20 bp, synthetic construct
  source 1..20: mol_type = other DNA
gcagctccttgatatcctgc

SEQ ID NO: 143 - DNA, 20 bp, synthetic construct
  source 1..20: mol_type = other DNA
agcagctccttgatatcctg

SEQ ID NO: 144 - DNA, 20 bp, synthetic construct
  source 1..20: mol_type = other DNA
cagcagctccttgatatcct

SEQ ID NO: 145 - DNA, 20 bp, synthetic construct
  source 1..20: mol_type = other DNA
ccagcagctccttgatatcc

SEQ ID NO: 146 - DNA, 20 bp, synthetic construct
  source 1..20: mol_type = other DNA
tccagcagctccttgatatc

SEQ ID NO: 147 - DNA, 20 bp, synthetic construct
  source 1..20: mol_type = other DNA
ctccagcagctccttgatat

SEQ ID NO: 148 - DNA, 20 bp, synthetic construct
  source 1..20: mol_type = other DNA
gctccagcagctccttgata

SEQ ID NO: 149 - DNA, 20 bp, synthetic construct
  source 1..20: mol_type = other DNA
ggctccagcagctccttgat

SEQ ID NO: 150 - DNA, 20 bp, synthetic construct
  source 1..20: mol_type = other DNA
agtgagggcagggcttagtc

SEQ ID NO: 151 - DNA, 20 bp, synthetic construct
  source 1..20: mol_type = other DNA
cagtgagggcagggcttagt

SEQ ID NO: 152 - DNA, 20 bp, synthetic construct
  source 1..20: mol_type = other DNA
tgcagtgagggcagggctta

SEQ ID NO: 153 - DNA, 20 bp, synthetic construct
  source 1..20: mol_type = other DNA
gttcggagggcaagagtgat

SEQ ID NO: 154 - DNA, 20 bp, synthetic construct
  source 1..20: mol_type = other DNA
cgttcggagggcaagagtga

SEQ ID NO: 155 - DNA, 20 bp, synthetic construct
  source 1..20: mol_type = other DNA
agcgttcggagggcaagagt

SEQ ID NO: 156 - DNA, 20 bp, synthetic construct
  source 1..20: mol_type = other DNA
aagcgttcggagggcaagag

SEQ ID NO: 157 - DNA, 20 bp, synthetic construct
  source 1..20: mol_type = other DNA
gggtcagagagtggccttca

SEQ ID NO: 158 - DNA, 20 bp, synthetic construct
  source 1..20: mol_type = other DNA
tgggtcagagagtggccttc

SEQ ID NO: 159 - DNA, 20 bp, synthetic construct
  source 1..20: mol_type = other DNA
ttgggtcagagagtggcctt

SEQ ID NO: 160 - DNA, 20 bp, synthetic construct
  source 1..20: mol_type = other DNA
attgggtcagagagtggcct

SEQ ID NO: 161 - DNA, 20 bp, synthetic construct
  source 1..20: mol_type = other DNA
aattgggtcagagagtggcc

SEQ ID NO: 162 - DNA, 20 bp, synthetic construct
  source 1..20: mol_type = other DNA
aaattgggtcagagagtggc

SEQ ID NO: 163 - DNA, 20 bp, synthetic construct
  source 1..20: mol_type = other DNA
caaattgggtcagagagtgg

SEQ ID NO: 164 - DNA, 20 bp, synthetic construct
  source 1..20: mol_type = other DNA
acaaattgggtcagagagtg

SEQ ID NO: 165 - DNA, 20 bp, synthetic construct
  source 1..20: mol_type = other DNA
aacaaattgggtcagagagt

SEQ ID NO: 166 - DNA, 20 bp, synthetic construct
  source 1..20: mol_type = other DNA
gaacaaattgggtcagagag

SEQ ID NO: 167 - DNA, 20 bp, synthetic construct
  source 1..20: mol_type = other DNA
gctcactgttcaggaaatga

SEQ ID NO: 168 - DNA, 20 bp, synthetic construct
  source 1..20: mol_type = other DNA
tgctcactgttcaggaaatg

SEQ ID NO: 169 - DNA, 20 bp, synthetic construct
  source 1..20: mol_type = other DNA
gtgctcactgttcaggaaat

SEQ ID NO: 170 - DNA, 20 bp, synthetic construct
  source 1..20: mol_type = other DNA
tgtgctcactgttcaggaaa

SEQ ID NO: 171 - DNA, 20 bp, synthetic construct
  source 1..20: mol_type = other DNA
ttgtgctcactgttcaggaa

SEQ ID NO: 172 - DNA, 20 bp, synthetic construct
  source 1..20: mol_type = other DNA
cttgtgctcactgttcagga

SEQ ID NO: 173 - DNA, 20 bp, synthetic construct
  source 1..20: mol_type = other DNA
ccttgtgctcactgttcagg

SEQ ID NO: 174 - DNA, 20 bp, synthetic construct
  source 1..20: mol_type = other DNA
tccttgtgctcactgttcag

SEQ ID NO: 175 - DNA, 20 bp, synthetic construct
  source 1..20: mol_type = other DNA
ctccttgtgctcactgttca

SEQ ID NO: 176 - DNA, 20 bp, synthetic construct
  source 1..20: mol_type = other DNA
tctccttgtgctcactgttc

SEQ ID NO: 177 - DNA, 20 bp, synthetic construct
  source 1..20: mol_type = other DNA
atctccttgtgctcactgtt

SEQ ID NO: 178 - DNA, 20 bp, synthetic construct
  source 1..20: mol_type = other DNA
gatctccttgtgctcactgt

SEQ ID NO: 179 - DNA, 20 bp, synthetic construct
  source 1..20: mol_type = other DNA
gaggtggatggagggtgtga

SEQ ID NO: 180 - DNA, 20 bp, synthetic construct
  source 1..20: mol_type = other DNA
tgaggtggatggagggtgtg

SEQ ID NO: 181 - DNA, 20 bp, synthetic construct
  source 1..20: mol_type = other DNA
atgaggtggatggagggtgt

SEQ ID NO: 182 - DNA, 20 bp, synthetic construct
  source 1..20: mol_type = other DNA
gatgaggtggatggagggtg

SEQ ID NO: 183 - DNA, 20 bp, synthetic construct
  source 1..20: mol_type = other DNA
tgatgaggtggatggagggt

SEQ ID NO: 184 - DNA, 20 bp, synthetic construct
  source 1..20: mol_type = other DNA
ctgatgaggtggatggaggg

SEQ ID NO: 185 - DNA, 20 bp, synthetic construct
  source 1..20: mol_type = other DNA
actgatgaggtggatggagg

SEQ ID NO: 186 - DNA, 20 bp, synthetic construct
  source 1..20: mol_type = other DNA
gtagcgaatgtcagggtcag

SEQ ID NO: 187 - DNA, 20 bp, synthetic construct
  source 1..20: mol_type = other DNA
agtagcgaatgtcagggtca

SEQ ID NO: 188 - DNA, 20 bp, synthetic construct
  source 1..20: mol_type = other DNA
cagtagcgaatgtcagggtc

SEQ ID NO: 189 - DNA, 20 bp, synthetic construct
  source 1..20: mol_type = other DNA
acagtagcgaatgtcagggt

SEQ ID NO: 190 - DNA, 20 bp, synthetic construct
  source 1..20: mol_type = other DNA
cacagtagcgaatgtcaggg

SEQ ID NO: 191 - DNA, 20 bp, synthetic construct
  source 1..20: mol_type = other DNA
ggttcatgctactgagtcgg

SEQ ID NO: 192 - DNA, 20 bp, synthetic construct
  source 1..20: mol_type = other DNA
gggttcatgctactgagtcg

SEQ ID NO: 193 - DNA, 20 bp, synthetic construct
  source 1..20: mol_type = other DNA
agggttcatgctactgagtc

SEQ ID NO: 194 - DNA, 20 bp, synthetic construct
  source 1..20: mol_type = other DNA
cagggttcatgctactgagt

SEQ ID NO: 195 - DNA, 20 bp, synthetic construct
  source 1..20: mol_type = other DNA
gcagggttcatgctactgag

SEQ ID NO: 196 - DNA, 20 bp, synthetic construct
  source 1..20: mol_type = other DNA
ggcagggttcatgctactga

SEQ ID NO: 197 - DNA, 20 bp, synthetic construct
  source 1..20: mol_type = other DNA
aggcagggttcatgctactg

SEQ ID NO: 198 - DNA, 20 bp, synthetic construct
  source 1..20: mol_type = other DNA
aaggcagggttcatgctact

SEQ ID NO: 199 - DNA, 20 bp, synthetic construct
  source 1..20: mol_type = other DNA
aaaggcagggttcatgctac

SEQ ID NO: 200 - DNA, 20 bp, synthetic construct
  source 1..20: mol_type = other DNA
caaaggcagggttcatgcta

SEQ ID NO: 201 - DNA, 20 bp, synthetic construct
  source 1..20: mol_type = other DNA
acaaaggcagggttcatgct

SEQ ID NO: 202 - DNA, 20 bp, synthetic construct
  source 1..20: mol_type = other DNA
gacaaaggcagggttcatgc

SEQ ID NO: 203 - DNA, 20 bp, synthetic construct
  source 1..20: mol_type = other DNA
gtttggatcagggtctggat

SEQ ID NO: 204 - DNA, 20 bp, synthetic construct
  source 1..20: mol_type = other DNA
ggtttggatcagggtctgga

SEQ ID NO: 205 - DNA, 20 bp, synthetic construct
  source 1..20: mol_type = other DNA
gggtttggatcagggtctgg

SEQ ID NO: 206 - DNA, 20 bp, synthetic construct
  source 1..20: mol_type = other DNA
tgggtttggatcagggtctg

SEQ ID NO: 207 - DNA, 20 bp, synthetic construct
  source 1..20: mol_type = other DNA
ctgggtttggatcagggtct

SEQ ID NO: 208 - DNA, 20 bp, synthetic construct
  source 1..20: mol_type = other DNA
cctgggtttggatcagggtc

SEQ ID NO: 209 - DNA, 20 bp, synthetic construct
  source 1..20: mol_type = other DNA
acctgggtttggatcagggt

SEQ ID NO: 210 - DNA, 20 bp, synthetic construct
  source 1..20: mol_type = other DNA
cacctgggtttggatcaggg

SEQ ID NO: 211 - DNA, 20 bp, synthetic construct
  source 1..20: mol_type = other DNA
acacctgggtttggatcagg

SEQ ID NO: 212 - DNA, 20 bp, synthetic construct
  source 1..20: mol_type = other DNA
gctcagtcttcagaaaattc

SEQ ID NO: 213 - DNA, 20 bp, synthetic construct
  source 1..20: mol_type = other DNA
ctgctcagtcttcagaaaat

SEQ ID NO: 214 - DNA, 20 bp, synthetic construct
  source 1..20: mol_type = other DNA
tctgctcagtcttcagaaaa

SEQ ID NO: 215 - DNA, 20 bp, synthetic construct
  source 1..20: mol_type = other DNA
ttctgctcagtcttcagaaa

SEQ ID NO: 216 - DNA, 20 bp, synthetic construct
  source 1..20: mol_type = other DNA
gttctgctcagtcttcagaa

SEQ ID NO: 217 - DNA, 20 bp, synthetic construct
  source 1..20: mol_type = other DNA
ggttctgctcagtcttcaga

SEQ ID NO: 218 - DNA, 20 bp, synthetic construct
  source 1..20: mol_type = other DNA
tggttctgctcagtcttcag

SEQ ID NO: 219 - DNA, 20 bp, synthetic construct
  source 1..20: mol_type = other DNA
ctggttctgctcagtcttca

SEQ ID NO: 220 - DNA, 20 bp, synthetic construct
  source 1..20: mol_type = other DNA
cctggttctgctcagtcttc

SEQ ID NO: 221 - DNA, 20 bp, synthetic construct
  source 1..20: mol_type = other DNA
ggcaagtttcccatgttgac

SEQ ID NO: 222 - DNA, 20 bp, synthetic construct
  source 1..20: mol_type = other DNA
aggcaagtttcccatgttga

SEQ ID NO: 223 - DNA, 20 bp, synthetic construct
  source 1..20: mol_type = other DNA
gaggcaagtttcccatgttg

SEQ ID NO: 224 - DNA, 20 bp, synthetic construct
  source 1..20: mol_type = other DNA
agaggcaagtttcccatgtt

SEQ ID NO: 225 - DNA, 20 bp, synthetic construct
  source 1..20: mol_type = other DNA
cagaggcaagtttcccatgt

SEQ ID NO: 226 - DNA, 20 bp, synthetic construct
  source 1..20: mol_type = other DNA
agagagtgactggtctcgga

SEQ ID NO: 227 - DNA, 20 bp, synthetic construct
  source 1..20: mol_type = other DNA
gagagagtgactggtctcgg

SEQ ID NO: 228 - DNA, 20 bp, synthetic construct
  source 1..20: mol_type = other DNA
atgagagagtgactggtctc

SEQ ID NO: 229 - DNA, 20 bp, synthetic construct
  source 1..20: mol_type = other DNA
gatgagagagtgactggtct

SEQ ID NO: 230 - DNA, 20 bp, synthetic construct
  source 1..20: mol_type = other DNA
tgatgagagagtgactggtc

SEQ ID NO: 231 - DNA, 20 bp, synthetic construct
  source 1..20: mol_type = other DNA
atgatgagagagtgactggt

SEQ ID NO: 232 - DNA, 20 bp, synthetic construct
  source 1..20: mol_type = other DNA
gatgatgagagagtgactgg

SEQ ID NO: 233 - DNA, 20 bp, synthetic construct
  source 1..20: mol_type = other DNA
tgatgatgagagagtgactg

SEQ ID NO: 234 - DNA, 20 bp, synthetic construct
  source 1..20: mol_type = other DNA
gtgatgatgagagagtgact

SEQ ID NO: 235 - DNA, 20 bp, synthetic construct
  source 1..20: mol_type = other DNA
tgtgatgatgagagagtgac

SEQ ID NO: 236 - DNA, 20 bp, synthetic construct
  source 1..20: mol_type = other DNA
gtgtgatgatgagagagtga

SEQ ID NO: 237 - DNA, 20 bp, synthetic construct
  source 1..20: mol_type = other DNA
ggtgtgatgatgagagagtg

SEQ ID NO: 238 - DNA, 20 bp, synthetic construct
  source 1..20: mol_type = other DNA
tggtgtgatgatgagagagt

SEQ ID NO: 239 - DNA, 20 bp, synthetic construct
  source 1..20: mol_type = other DNA
atggtgtgatgatgagagag

SEQ ID NO: 240 - DNA, 20 bp, synthetic construct
  source 1..20: mol_type = other DNA
catggtgtgatgatgagaga

SEQ ID NO: 241 - DNA, 20 bp, synthetic construct
  source 1..20: mol_type = other DNA
tggacaaccatggtgtgatg

SEQ ID NO: 242 - DNA, 20 bp, synthetic construct
  source 1..20: mol_type = other DNA
aggtgatggcctggacaacc

SEQ ID NO: 243 - DNA, 20 bp, synthetic construct
  source 1..20: mol_type = other DNA
aaggtgatggcctggacaac

SEQ ID NO: 244 - DNA, 20 bp, synthetic construct
  source 1..20: mol_type = other DNA
gaaggtgatggcctggacaa

SEQ ID NO: 245 - DNA, 20 bp, synthetic construct
  source 1..20: mol_type = other DNA
tgaaggtgatggcctggaca

SEQ ID NO: 246 - DNA, 20 bp, synthetic construct
  source 1..20: mol_type = other DNA
atgaaggtgatggcctggac

SEQ ID NO: 247 - DNA, 20 bp, synthetic construct
  source 1..20: mol_type = other DNA
gatgaaggtgatggcctgga

SEQ ID NO: 248 - DNA, 20 bp, synthetic construct
  source 1..20: mol_type = other DNA
agatgaaggtgatggcctgg

SEQ ID NO: 249 - DNA, 20 bp, synthetic construct
  source 1..20: mol_type = other DNA
gaagatgaaggtgatggcct

SEQ ID NO: 250 - DNA, 20 bp, synthetic construct
  source 1..20: mol_type = other DNA
tgaagatgaaggtgatggcc

SEQ ID NO: 251 - DNA, 20 bp, synthetic construct
  source 1..20: mol_type = other DNA
ttgaagatgaaggtgatggc

SEQ ID NO: 252 - DNA, 20 bp, synthetic construct
  source 1..20: mol_type = other DNA
cttgaagatgaaggtgatgg

SEQ ID NO: 253 - DNA, 20 bp, synthetic construct
  source 1..20: mol_type = other DNA
acttgaagatgaaggtgatg

SEQ ID NO: 254 - DNA, 20 bp, synthetic construct
  source 1..20: mol_type = other DNA
gacttgaagatgaaggtgat

SEQ ID NO: 255 - DNA, 20 bp, synthetic construct
  source 1..20: mol_type = other DNA
ggacttgaagatgaaggtga

SEQ ID NO: 256 - DNA, 20 bp, synthetic construct
  source 1..20: mol_type = other DNA
gggacttgaagatgaaggtg

SEQ ID NO: 257 - DNA, 20 bp, synthetic construct
  source 1..20: mol_type = other DNA
agggacttgaagatgaaggt

SEQ ID NO: 258 - DNA, 20 bp, synthetic construct
  source 1..20: mol_type = other DNA
cagggacttgaagatgaagg

SEQ ID NO: 259 - DNA, 20 bp, synthetic construct
  source 1..20: mol_type = other DNA
atgacctggggcaggaactg

SEQ ID NO: 260 - DNA, 20 bp, synthetic construct
  source 1..20: mol_type = other DNA
catgacctggggcaggaact

SEQ ID NO: 261 - DNA, 20 bp, synthetic construct
  source 1..20: mol_type = other DNA
cgaatgacgttaaggaacgt

SEQ ID NO: 262 - DNA, 20 bp, synthetic construct
  source 1..20: mol_type = other DNA
tcgaatgacgttaaggaacg

SEQ ID NO: 263 - DNA, 20 bp, synthetic construct
  source 1..20: mol_type = other DNA
ctcgaatgacgttaaggaac

SEQ ID NO: 264 - DNA, 20 bp, synthetic construct
  source 1..20: mol_type = other DNA
actcgaatgacgttaaggaa

SEQ ID NO: 265 - DNA, 20 bp, synthetic construct
  source 1..20: mol_type = other DNA
gactcgaatgacgttaagga

SEQ ID NO: 266 - DNA, 20 bp, synthetic construct
  source 1..20: mol_type = other DNA
agactcgaatgacgttaagg

SEQ ID NO: 267 - DNA, 20 bp, synthetic construct
  source 1..20: mol_type = other DNA
gcatgtgtgggatcagctgg

SEQ ID NO: 268 - DNA, 20 bp, synthetic construct
  source 1..20: mol_type = other DNA
agcatgtgtgggatcagctg

SEQ ID NO: 269 - DNA, 20 bp, synthetic construct
  source 1..20: mol_type = other DNA
cagcatgtgtgggatcagct

SEQ ID NO: 270 - DNA, 20 bp, synthetic construct
  source 1..20: mol_type = other DNA
gcagcatgtgtgggatcagc

SEQ ID NO: 271 - DNA, 20 bp, synthetic construct
  source 1..20: mol_type = other DNA
cgcagcatgtgtgggatcag

SEQ ID NO: 272 - DNA, 20 bp, synthetic construct
  source 1..20: mol_type = other DNA
acgcagcatgtgtgggatca

SEQ ID NO: 273 - DNA, 20 bp, synthetic construct
  source 1..20: mol_type = other DNA
cacgcagcatgtgtgggatc

SEQ ID NO: 274 - DNA, 20 bp, synthetic construct
  source 1..20: mol_type = other DNA
acacgcagcatgtgtgggat

SEQ ID NO: 275 - DNA, 20 bp, synthetic construct
  source 1..20: mol_type = other DNA
gacacgcagcatgtgtggga

SEQ ID NO: 276 - DNA, 20 bp, synthetic construct
  source 1..20: mol_type = other DNA
agacacgcagcatgtgtggg

SEQ ID NO: 277 - DNA, 20 bp, synthetic construct
  source 1..20: mol_type = other DNA
ggagcttcaggggcatcaaa

SEQ ID NO: 278 - DNA, 20 bp, synthetic construct
  source 1..20: mol_type = other DNA
tggagcttcaggggcatcaa

SEQ ID NO: 279 - DNA, 20 bp, synthetic construct
  source 1..20: mol_type = other DNA
gtggagcttcaggggcatca

SEQ ID NO: 280 - DNA, 20 bp, synthetic construct
  source 1..20: mol_type = other DNA
agtggagcttcaggggcatc

SEQ ID NO: 281 - DNA, 20 bp, synthetic construct
  source 1..20: mol_type = other DNA
cagtggagcttcaggggcat

SEQ ID NO: 282 - DNA, 20 bp, synthetic construct
  source 1..20: mol_type = other DNA
atggcagtggagcttcaggg

SEQ ID NO: 283 - DNA, 20 bp, synthetic construct
  source 1..20: mol_type = other DNA
gatggcagtggagcttcagg

SEQ ID NO: 284 - DNA, 20 bp, synthetic construct
  source 1..20: mol_type = other DNA
gagatggcagtggagcttca

SEQ ID NO: 285 - DNA, 20 bp, synthetic construct
  source 1..20: mol_type = other DNA
cgagatggcagtggagcttc

SEQ ID NO: 286 - DNA, 20 bp, synthetic construct
  source 1..20: mol_type = other DNA
tcgagatggcagtggagctt

SEQ ID NO: 287 - DNA, 20 bp, synthetic construct
  source 1..20: mol_type = other DNA
ttcgagatggcagtggagct

SEQ ID NO: 288 - DNA, 20 bp, synthetic construct
  source 1..20: mol_type = other DNA
tttcgagatggcagtggagc

SEQ ID NO: 289 - DNA, 20 bp, synthetic construct
  source 1..20: mol_type = other DNA
ctttcgagatggcagtggag

SEQ ID NO: 290 - DNA, 20 bp, synthetic construct
  source 1..20: mol_type = other DNA
cctttcgagatggcagtgga

SEQ ID NO: 291 - DNA, 20 bp, synthetic construct
  source 1..20: mol_type = other DNA
ggaggcatagtcagtgaaat

SEQ ID NO: 292 - DNA, 20 bp, synthetic construct
  source 1..20: mol_type = other DNA
gggaggcatagtcagtgaaa

SEQ ID NO: 293 - DNA, 20 bp, synthetic construct
  source 1..20: mol_type = other DNA
cgggaggcatagtcagtgaa

SEQ ID NO: 294 - DNA, 20 bp, synthetic construct
  source 1..20: mol_type = other DNA
ccgggaggcatagtcagtga

SEQ ID NO: 295 - DNA, 20 bp, synthetic construct
  source 1..20: mol_type = other DNA
tccgggaggcatagtcagtg

SEQ ID NO: 296 - DNA, 20 bp, synthetic construct
  source 1..20: mol_type = other DNA
atccgggaggcatagtcagt

SEQ ID NO: 297 - DNA, 20 bp, synthetic construct
  source 1..20: mol_type = other DNA
gatccgggaggcatagtcag

SEQ ID NO: 298 - DNA, 20 bp, synthetic construct
  source 1..20: mol_type = other DNA
agggtgaatgatccgggagg

SEQ ID NO: 299 - DNA, 20 bp, synthetic construct
  source 1..20: mol_type = other DNA
tagggtgaatgatccgggag

SEQ ID NO: 300 - DNA, 20 bp, synthetic construct
  source 1..20: mol_type = other DNA
atagggtgaatgatccggga

SEQ ID NO: 301 - DNA, 20 bp, synthetic construct
  source 1..20: mol_type = other DNA
aatagggtgaatgatccggg

SEQ ID NO: 302 - DNA, 20 bp, synthetic construct
  source 1..20: mol_type = other DNA
atcgggttgtaggcctgtgc

SEQ ID NO: 303 - DNA, 20 bp, synthetic construct
  source 1..20: mol_type = other DNA
catcgggttgtaggcctgtg

SEQ ID NO: 304 - DNA, 20 bp, synthetic construct
  source 1..20: mol_type = other DNA
ccatcgggttgtaggcctgt

SEQ ID NO: 305 - DNA, 20 bp, synthetic construct
  source 1..20: mol_type = other DNA
tcattcagttcagaccagca

SEQ ID NO: 306 - DNA, 20 bp, synthetic construct
  source 1..20: mol_type = other DNA
ttcattcagttcagaccagc

SEQ ID NO: 307 - DNA, 20 bp, synthetic construct
  source 1..20: mol_type = other DNA
cttcattcagttcagaccag

SEQ ID NO: 308 - DNA, 20 bp, synthetic construct
  source 1..20: mol_type = other DNA
tcttcattcagttcagacca

SEQ ID NO: 309 - DNA, 20 bp, synthetic construct
  source 1..20: mol_type = other DNA
atcttcattcagttcagacc

SEQ ID NO: 310 - DNA, 20 bp, synthetic construct
  source 1..20: mol_type = other DNA
gatcttcattcagttcagac

SEQ ID NO: 311 - DNA, 20 bp, synthetic construct
  source 1..20: mol_type = other DNA
gtgacttcagcgatgtcttg

SEQ ID NO: 312 - DNA, 20 bp, synthetic construct
  source 1..20: mol_type = other DNA
tgtgacttcagcgatgtctt

SEQ ID NO: 313 - DNA, 20 bp, synthetic construct
  source 1..20: mol_type = other DNA
gtgtgacttcagcgatgtct

SEQ ID NO: 314 - DNA, 20 bp, synthetic construct
  source 1..20: mol_type = other DNA
tgtgtgacttcagcgatgtc

SEQ ID NO: 315 - DNA, 20 bp, synthetic construct
  source 1..20: mol_type = other DNA
ctgtgtgacttcagcgatgt

SEQ ID NO: 316 - DNA, 20 bp, synthetic construct
  source 1..20: mol_type = other DNA
ggtctgtgtgacttcagcga

SEQ ID NO: 317 - DNA, 20 bp, synthetic construct
  source 1..20: mol_type = other DNA
gggtctgtgtgacttcagcg

SEQ ID NO: 318 - DNA, 20 bp, synthetic construct
  source 1..20: mol_type = other DNA
agggtctgtgtgacttcagc

SEQ ID NO: 319 - DNA, 20 bp, synthetic construct
  source 1..20: mol_type = other DNA
gagggtctgtgtgacttcag

SEQ ID NO: 320 - DNA, 20 bp, synthetic construct
  source 1..20: mol_type = other DNA
agagggtctgtgtgacttca

SEQ ID NO: 321 - DNA, 20 bp, synthetic construct
  source 1..20: mol_type = other DNA
aagagggtctgtgtgacttc

SEQ ID NO: 322 - DNA, 20 bp, synthetic construct
  source 1..20: mol_type = other DNA
taagagggtctgtgtgactt

SEQ ID NO: 323 - DNA, 20 bp, synthetic construct
  source 1..20: mol_type = other DNA
ttaagagggtctgtgtgact

SEQ ID NO: 324 - DNA, 20 bp, synthetic construct
  source 1..20: mol_type = other DNA
tttaagagggtctgtgtgac

SEQ ID NO: 325 - DNA, 20 bp, synthetic construct
  source 1..20: mol_type = other DNA
gtttaagagggtctgtgtga

SEQ ID NO: 326 - DNA, 20 bp, synthetic construct
  source 1..20: mol_type = other DNA
agtttaagagggtctgtgtg

SEQ ID NO: 327 - DNA, 20 bp, synthetic construct
  source 1..20: mol_type = other DNA
aagtttaagagggtctgtgt

SEQ ID NO: 328 - DNA, 20 bp, synthetic construct
  source 1..20: mol_type = other DNA
caagtttaagagggtctgtg

SEQ ID NO: 329 - DNA, 20 bp, synthetic construct
  source 1..20: mol_type = other DNA
ccaagtttaagagggtctgt

SEQ ID NO: 330 - DNA, 20 bp, synthetic construct
  source 1..20: mol_type = other DNA
gccaagtttaagagggtctg

SEQ ID NO: 331 - DNA, 20 bp, synthetic construct
  source 1..20: mol_type = other DNA
agccaagtttaagagggtct

SEQ ID NO: 332 - DNA, 20 bp, synthetic construct
  source 1..20: mol_type = other DNA
cagccaagtttaagagggtc

SEQ ID NO: 333 - DNA, 20 bp, synthetic construct
  source 1..20: mol_type = other DNA
tcagccaagtttaagagggt

SEQ ID NO: 334 - DNA, 20 bp, synthetic construct
  source 1..20: mol_type = other DNA
ttcagccaagtttaagaggg

SEQ ID NO: 335 - DNA, 20 bp, synthetic construct
  source 1..20: mol_type = other DNA
tgtagtgctttggcatatgc

SEQ ID NO: 336 - DNA, 20 bp, synthetic construct
  source 1..20: mol_type = other DNA
gtgtagtgctttggcatatg

SEQ ID NO: 337 - DNA, 20 bp, synthetic construct
  source 1..20: mol_type = other DNA
agtgtagtgctttggcatat

SEQ ID NO: 338 - DNA, 20 bp, synthetic construct
  source 1..20: mol_type = other DNA
tagtgtagtgctttggcata

SEQ ID NO: 339 - DNA, 20 bp, synthetic construct
  source 1..20: mol_type = other DNA
gtagtgtagtgctttggcat

SEQ ID NO: 340 - DNA, 20 bp, synthetic construct
  source 1..20: mol_type = other DNA
tgtagtgtagtgctttggca

SEQ ID NO: 341 - DNA, 20 bp, synthetic construct
  source 1..20: mol_type = other DNA
ttgtagtgtagtgctttggc

SEQ ID NO: 342 - DNA, 20 bp, synthetic construct
  source 1..20: mol_type = other DNA
tttgtagtgtagtgctttgg

SEQ ID NO: 343 - DNA, 20 bp, synthetic construct
  source 1..20: mol_type = other DNA
ctttgtagtgtagtgctttg

SEQ ID NO: 344 - DNA, 20 bp, synthetic construct
  source 1..20: mol_type = other DNA
gttctttgtagtgtagtgct

SEQ ID NO: 345 - DNA, 20 bp, synthetic construct
  source 1..20: mol_type = other DNA
agttctttgtagtgtagtgc

SEQ ID NO: 346 - DNA, 20 bp, synthetic construct
  source 1..20: mol_type = other DNA
cagttctttgtagtgtagtg

SEQ ID NO: 347 - DNA, 20 bp, synthetic construct
  source 1..20: mol_type = other DNA
ccagttctttgtagtgtagt

SEQ ID NO: 348 - DNA, 20 bp, synthetic construct
  source 1..20: mol_type = other DNA
tccagttctttgtagtgtag

SEQ ID NO: 349 - DNA, 20 bp, synthetic construct
  source 1..20: mol_type = other DNA
ctccagttctttgtagtgta

SEQ ID NO: 350 - DNA, 20 bp, synthetic construct
  source 1..20: mol_type = other DNA
ggcatattctaacactccgg

SEQ ID NO: 351 - DNA, 20 bp, synthetic construct
  source 1..20: mol_type = other DNA
tggcatattctaacactccg

SEQ ID NO: 352 - DNA, 20 bp, synthetic construct
  source 1..20: mol_type = other DNA
atggcatattctaacactcc

SEQ ID NO: 353 - DNA, 20 bp, synthetic construct
  source 1..20: mol_type = other DNA
catggcatattctaacactc

SEQ ID NO: 354 - DNA, 20 bp, synthetic construct
  source 1..20: mol_type = other DNA
ctccaaagtgtttcatggca

SEQ ID NO: 355 - DNA, 20 bp, synthetic construct
  source 1..20: mol_type = other DNA
tctccaaagtgtttcatggc

SEQ ID NO: 356 - DNA, 20 bp, synthetic construct
  source 1..20: mol_type = other DNA
tgcagtttctcataccaggt

SEQ ID NO: 357 - DNA, 20 bp, synthetic construct
  source 1..20: mol_type = other DNA
gtgcagtttctcataccagg

SEQ ID NO: 358 - DNA, 20 bp, synthetic construct
  source 1..20: mol_type = other DNA
cgtgcagtttctcataccag

SEQ ID NO: 359 - DNA, 20 bp, synthetic construct
  source 1..20: mol_type = other DNA
tcgtgcagtttctcatacca

SEQ ID NO: 360 - DNA, 20 bp, synthetic construct
  source 1..20: mol_type = other DNA
ctcgtgcagtttctcatacc

SEQ ID NO: 361 - DNA, 20 bp, synthetic construct
  source 1..20: mol_type = other DNA
actcgtgcagtttctcatac

SEQ ID NO: 362 - DNA, 20 bp, synthetic construct
  source 1..20: mol_type = other DNA
cactcgtgcagtttctcata

SEQ ID NO: 363 - DNA, 20 bp, synthetic construct
  source 1..20: mol_type = other DNA
ccactcgtgcagtttctcat

SEQ ID NO: 364 - DNA, 20 bp, synthetic construct
  source 1..20: mol_type = other DNA
cccactcgtgcagtttctca

SEQ ID NO: 365 - DNA, 20 bp, synthetic construct
  source 1..20: mol_type = other DNA
tcccactcgtgcagtttctc

SEQ ID NO: 366 - DNA, 20 bp, synthetic construct
  source 1..20: mol_type = other DNA
ctcccactcgtgcagtttct

SEQ ID NO: 367 - DNA, 20 bp, synthetic construct
  source 1..20: mol_type = other DNA
cctcccactcgtgcagtttc

SEQ ID NO: 368 - DNA, 20 bp, synthetic construct
  source 1..20: mol_type = other DNA
tcctcccactcgtgcagttt

SEQ ID NO: 369 - DNA, 20 bp, synthetic construct
  source 1..20: mol_type = other DNA
atcctcccactcgtgcagtt

SEQ ID NO: 370 - DNA, 20 bp, synthetic construct
  source 1..20: mol_type = other DNA
catcctcccactcgtgcagt

SEQ ID NO: 371 - DNA, 20 bp, synthetic construct
  source 1..20: mol_type = other DNA
gtccattttcttgtcatagg

SEQ ID NO: 372 - DNA, 20 bp, synthetic construct
  source 1..20: mol_type = other DNA
ggtgtccattttcttgtcat

SEQ ID NO: 373 - DNA, 20 bp, synthetic construct
  source 1..20: mol_type = other DNA
tggtgtccattttcttgtca

SEQ ID NO: 374 - DNA, 20 bp, synthetic construct
  source 1..20: mol_type = other DNA
ttggtgtccattttcttgtc

SEQ ID NO: 375 - DNA, 20 bp, synthetic construct
  source 1..20: mol_type = other DNA
gttggtgtccattttcttgt

SEQ ID NO: 376 - DNA, 20 bp, synthetic construct
  source 1..20: mol_type = other DNA
tgttggtgtccattttcttg

SEQ ID NO: 377 - DNA, 20 bp, synthetic construct
  source 1..20: mol_type = other DNA
cttgttggtgtccattttct

SEQ ID NO: 378 - DNA, 20 bp, synthetic construct
  source 1..20: mol_type = other DNA
ccttgttggtgtccattttc

SEQ ID NO: 379 - DNA, 20 bp, synthetic construct
  source 1..20: mol_type = other DNA
gtctcatcattaaccagggt

SEQ ID NO: 380 - DNA, 20 bp, synthetic construct
  source 1..20: mol_type = other DNA
ggtctcatcattaaccaggg

SEQ ID NO: 381 - DNA, 20 bp, synthetic construct
  source 1..20: mol_type = other DNA
gggtctcatcattaaccagg

SEQ ID NO: 382 - DNA, 20 bp, synthetic construct
  source 1..20: mol_type = other DNA
tgggtctcatcattaaccag

SEQ ID NO: 383 - DNA, 20 bp, synthetic construct
  source 1..20: mol_type = other DNA
ttgggtctcatcattaacca

SEQ ID NO: 384 - DNA, 20 bp, synthetic construct
  source 1..20: mol_type = other DNA
cttgggtctcatcattaacc

SEQ ID NO: 385 - DNA, 20 bp, synthetic construct
  source 1..20: mol_type = other DNA
gcttgggtctcatcattaac

SEQ ID NO: 386 - DNA, 20 bp, synthetic construct
  source 1..20: mol_type = other DNA
ttggcttgggtctcatcatt

SEQ ID NO: 387 - DNA, 20 bp, synthetic construct
  source 1..20: mol_type = other DNA
cttggcttgggtctcatcat

SEQ ID NO: 388 - DNA, 20 bp, synthetic construct
  source 1..20: mol_type = other DNA
tcttggcttgggtctcatca

SEQ ID NO: 389 - DNA, 20 bp, synthetic construct
  source 1..20: mol_type = other DNA
tttgtactggataacctcct

SEQ ID NO: 390 - DNA, 20 bp, synthetic construct
  source 1..20: mol_type = other DNA
gtttgtactggataacctcc

SEQ ID NO: 391 - DNA, 20 bp, synthetic construct
  source 1..20: mol_type = other DNA
agtttgtactggataacctc

SEQ ID NO: 392 - DNA, 20 bp, synthetic construct
  source 1..20: mol_type = other DNA
ggatggtcaagttgccgaga

SEQ ID NO: 393 - DNA, 20 bp, synthetic construct
  source 1..20: mol_type = other DNA
aggatggtcaagttgccgag

SEQ ID NO: 394 - DNA, 20 bp, synthetic construct
  source 1..20: mol_type = other DNA
gaggatggtcaagttgccga

SEQ ID NO: 395 - DNA, 20 bp, synthetic construct
  source 1..20: mol_type = other DNA
agaggatggtcaagttgccg

SEQ ID NO: 396 - DNA, 20 bp, synthetic construct
  source 1..20: mol_type = other DNA
cagaggatggtcaagttgcc

SEQ ID NO: 397 - DNA, 20 bp, synthetic construct
  source 1..20: mol_type = other DNA
gcagaggatggtcaagttgc

SEQ ID NO: 398 - DNA, 20 bp, synthetic construct
  source 1..20: mol_type = other DNA
ggcagaggatggtcaagttg

SEQ ID NO: 399 - DNA, 20 bp, synthetic construct
  source 1..20: mol_type = other DNA
tggcagaggatggtcaagtt

SEQ ID NO: 400 - DNA, 20 bp, synthetic construct
  source 1..20: mol_type = other DNA
ttggcagaggatggtcaagt

SEQ ID NO: 401 - DNA, 20 bp, synthetic construct
  source 1..20: mol_type = other DNA
gttggcagaggatggtcaag

SEQ ID NO: 402 - DNA, 20 bp, synthetic construct
  source 1..20: mol_type = other DNA
tgttggcagaggatggtcaa

SEQ ID NO: 403 - DNA, 20 bp, synthetic construct
  source 1..20: mol_type = other DNA
ctgttggcagaggatggtca

SEQ ID NO: 404 - DNA, 20 bp, synthetic construct
  source 1..20: mol_type = other DNA
actgttggcagaggatggtc

SEQ ID NO: 405 - DNA, 20 bp, synthetic construct
  source 1..20: mol_type = other DNA
aactgttggcagaggatggt

SEQ ID NO: 406 - DNA, 20 bp, synthetic construct
  source 1..20: mol_type = other DNA
gaactgttggcagaggatgg

SEQ ID NO: 407 - DNA, 20 bp, synthetic construct
  source 1..20: mol_type = other DNA
tgaactgttggcagaggatg

SEQ ID NO: 408 - DNA, 20 bp, synthetic construct
  source 1..20: mol_type = other DNA
gtgaactgttggcagaggat

SEQ ID NO: 409 - DNA, 20 bp, synthetic construct
  source 1..20: mol_type = other DNA
ggtgaactgttggcagagga

SEQ ID NO: 410 - DNA, 20 bp, synthetic construct
  source 1..20: mol_type = other DNA
gggtgaactgttggcagagg

SEQ ID NO: 411 - DNA, 20 bp, synthetic construct
  source 1..20: mol_type = other DNA
agggtgaactgttggcagag

SEQ ID NO: 412 - DNA, 20 bp, synthetic construct
  source 1..20: mol_type = other DNA
gagggtgaactgttggcaga

SEQ ID NO: 413 - DNA, 20 bp, synthetic construct
  source 1..20: mol_type = other DNA
tgagggtgaactgttggcag

SEQ ID NO: 414 - DNA, 20 bp, synthetic construct
  source 1..20: mol_type = other DNA
ctgagggtgaactgttggca

SEQ ID NO: 415 - DNA, 20 bp, synthetic construct
  source 1..20: mol_type = other DNA
ctgcatggtctggacaaaat

SEQ ID NO: 416 - DNA, 20 bp, synthetic construct
  source 1..20: mol_type = other DNA
gctgcatggtctggacaaaa

SEQ ID NO: 417 - DNA, 20 bp, synthetic construct
  source 1..20: mol_type = other DNA
tgctgcatggtctggacaaa

SEQ ID NO: 418 - DNA, 20 bp, synthetic construct
  source 1..20: mol_type = other DNA
ttgctgcatggtctggacaa

SEQ ID NO: 419 - DNA, 20 bp, synthetic construct
  source 1..20: mol_type = other DNA
gttgctgcatggtctggaca

SEQ ID NO: 420 - DNA, 20 bp, synthetic construct
  source 1..20: mol_type = other DNA
tgttgctgcatggtctggac

SEQ ID NO: 421 - DNA, 20 bp, synthetic construct
  source 1..20: mol_type = other DNA
ctgttgctgcatggtctgga

SEQ ID NO: 422 - DNA, 20 bp, synthetic construct
  source 1..20: mol_type = other DNA
ggattgtgctctcattgatg

SEQ ID NO: 423 - DNA, 20 bp, synthetic construct
  source 1..20: mol_type = other DNA
gggattgtgctctcattgat

SEQ ID NO: 424 - DNA, 20 bp, synthetic construct
  source 1..20: mol_type = other DNA
ggggattgtgctctcattga

SEQ ID NO: 425 - DNA, 20 bp, synthetic construct
  source 1..20: mol_type = other DNA
tggggattgtgctctcattg

SEQ ID NO: 426 - DNA, 20 bp, synthetic construct
  source 1..20: mol_type = other DNA
ttggggattgtgctctcatt

SEQ ID NO: 427 - DNA, 20 bp, synthetic construct
  source 1..20: mol_type = other DNA
tttggggattgtgctctcat

SEQ ID NO: 428 - DNA, 20 bp, synthetic construct
  source 1..20: mol_type = other DNA
ctttggggattgtgctctca

SEQ ID NO: 429 - DNA, 20 bp, synthetic construct
  source 1..20: mol_type = other DNA
actttggggattgtgctctc

SEQ ID NO: 430 - DNA, 20 bp, synthetic construct
  source 1..20: mol_type = other DNA
cactttggggattgtgctct

SEQ ID NO: 431 - DNA, 20 bp, synthetic construct
  source 1..20: mol_type = other DNA
gcactttggggattgtgctc

SEQ ID NO: 432 - DNA, 20 bp, synthetic construct
  source 1..20: mol_type = other DNA
agcactttggggattgtgct

SEQ ID NO: 433 - DNA, 20 bp, synthetic construct
  source 1..20: mol_type = other DNA
cagcactttggggattgtgc

SEQ ID NO: 434 - DNA, 20 bp, synthetic construct
  source 1..20: mol_type = other DNA
gcagcactttggggattgtg

SEQ ID NO: 435 - DNA, 20 bp, synthetic construct
  source 1..20: mol_type = other DNA
tgcagcactttggggattgt

SEQ ID NO: 436 - DNA, 20 bp, synthetic construct
  source 1..20: mol_type = other DNA
ctgcagcactttggggattg

SEQ ID NO: 437 - DNA, 20 bp, synthetic construct
  source 1..20: mol_type = other DNA
actgcagcactttggggatt

SEQ ID NO: 438 - DNA, 20 bp, synthetic construct
  source 1..20: mol_type = other DNA
tgtagtgtagcacagcttcg

SEQ ID NO: 439 - DNA, 20 bp, synthetic construct
  source 1..20: mol_type = other DNA
ttgtagtgtagcacagcttc

SEQ ID NO: 440 - DNA, 20 bp, synthetic construct
  source 1..20: mol_type = other DNA
tttgtagtgtagcacagctt

SEQ ID NO: 441 - DNA, 20 bp, synthetic construct
  source 1..20: mol_type = other DNA
gtttgtagtgtagcacagct

SEQ ID NO: 442 - DNA, 20 bp, synthetic construct
  source 1..20: mol_type = other DNA
tgtttgtagtgtagcacagc

SEQ ID NO: 443 - DNA, 20 bp, synthetic construct
  source 1..20: mol_type = other DNA
atgtttgtagtgtagcacag

SEQ ID NO: 444 - DNA, 20 bp, synthetic construct
  source 1..20: mol_type = other DNA
gatgtttgtagtgtagcaca

SEQ ID NO: 445 - DNA, 20 bp, synthetic construct
  source 1..20: mol_type = other DNA
tgatgtttgtagtgtagcac

SEQ ID NO: 446 - DNA, 20 bp, synthetic construct
  source 1..20: mol_type = other DNA
ctgatgtttgtagtgtagca

SEQ ID NO: 447 - DNA, 20 bp, synthetic construct
  source 1..20: mol_type = other DNA
tctgatgtttgtagtgtagc

SEQ ID NO: 448 - DNA, 20 bp, synthetic construct
  source 1..20: mol_type = other DNA
ggttctgatgtttgtagtgt

SEQ ID NO: 449 - DNA, 20 bp, synthetic construct
  source 1..20: mol_type = other DNA
tggttctgatgtttgtagtg

SEQ ID NO: 450 - DNA, 20 bp, synthetic construct
  source 1..20: mol_type = other DNA
cttggttctgatgtttgtag

SEQ ID NO: 451 - DNA, 20 bp, synthetic construct
  source 1..20: mol_type = other DNA
gcttggttctgatgtttgta

SEQ ID NO: 452 - DNA, 20 bp, synthetic construct
  source 1..20: mol_type = other DNA
ggcttggttctgatgtttgt

SEQ ID NO: 453 - DNA, 20 bp, synthetic construct
  source 1..20: mol_type = other DNA
gggcttggttctgatgtttg

SEQ ID NO: 454 - DNA, 20 bp, synthetic construct
  source 1..20: mol_type = other DNA
cgggcttggttctgatgttt

SEQ ID NO: 455 - DNA, 20 bp, synthetic construct
  source 1..20: mol_type = other DNA
gcgggcttggttctgatgtt

SEQ ID NO: 456 - DNA, 20 bp, synthetic construct
  source 1..20: mol_type = other DNA
cgcgggcttggttctgatgt

SEQ ID NO: 457 - DNA, 20 bp, synthetic construct
  source 1..20: mol_type = other DNA
tcgcgggcttggttctgatg

SEQ ID NO: 458 - DNA, 20 bp, synthetic construct
  source 1..20: mol_type = other DNA
atcgcgggcttggttctgat

SEQ ID NO: 459 - DNA, 20 bp, synthetic construct
  source 1..20: mol_type = other DNA
catcgcgggcttggttctga

SEQ ID NO: 460 - DNA, 20 bp, synthetic construct
  source 1..20: mol_type = other DNA
tcatcgcgggcttggttctg

SEQ ID NO: 461 - DNA, 20 bp, synthetic construct
  source 1..20: mol_type = other DNA
ctcatcgcgggcttggttct

SEQ ID NO: 462 - DNA, 20 bp, synthetic construct
  source 1..20: mol_type = other DNA
tctcatcgcgggcttggttc

SEQ ID NO: 463 - DNA, 20 bp, synthetic construct
  source 1..20: mol_type = other DNA
ttctcatcgcgggcttggtt

SEQ ID NO: 464 - DNA, 20 bp, synthetic construct
  source 1..20: mol_type = other DNA
cttctcatcgcgggcttggt

SEQ ID NO: 465 - DNA, 20 bp, synthetic construct
  source 1..20: mol_type = other DNA
gtatcctggaggttgttgcc

SEQ ID NO: 466 - DNA, 20 bp, synthetic construct
  source 1..20: mol_type = other DNA
tgtatcctggaggttgttgc

SEQ ID NO: 467 - DNA, 20 bp, synthetic construct
  source 1..20: mol_type = other DNA
gtgtatcctggaggttgttg

SEQ ID NO: 468 - DNA, 20 bp, synthetic construct
  source 1..20: mol_type = other DNA
agtgtatcctggaggttgtt

SEQ ID NO: 469 - DNA, 20 bp, synthetic construct
  source 1..20: mol_type = other DNA
gagtgtatcctggaggttgt

SEQ ID NO: 470 - DNA, 20 bp, synthetic construct
  source 1..20: mol_type = other DNA
ttgcaatgagctgaggtata

SEQ ID NO: 471 - DNA, 20 bp, synthetic construct
  source 1..20: mol_type = other DNA
gtgagaagctggtgaatgag

SEQ ID NO: 472 - DNA, 20 bp, synthetic construct
  source 1..20: mol_type = other DNA
tgtgagaagctggtgaatga

SEQ ID NO: 473 - DNA, 20 bp, synthetic construct
  source 1..20: mol_type = other DNA
ctgtgagaagctggtgaatg

SEQ ID NO: 474 - DNA, 20 bp, synthetic construct
  source 1..20: mol_type = other DNA
tctgtgagaagctggtgaat

SEQ ID NO: 475 - DNA, 20 bp, synthetic construct
  source 1..20: mol_type = other DNA
gtctgtgagaagctggtgaa

SEQ ID NO: 476 - DNA, 20 bp, synthetic construct
  source 1..20: mol_type = other DNA
tgtctgtgagaagctggtga

SEQ ID NO: 477 - DNA, 20 bp, synthetic construct
  source 1..20: mol_type = other DNA
atgtctgtgagaagctggtg

SEQ ID NO: 478 - DNA, 20 bp, synthetic construct
  source 1..20: mol_type = other DNA
aatgtctgtgagaagctggt

SEQ ID NO: 479 - DNA, 20 bp, synthetic construct
  source 1..20: mol_type = other DNA
caatgtctgtgagaagctgg

SEQ ID NO: 480 - DNA, 20 bp, synthetic construct
  source 1..20: mol_type = other DNA
ccaatgtctgtgagaagctg

SEQ ID NO: 481 - DNA, 20 bp, synthetic construct
  source 1..20: mol_type = other DNA
accaatgtctgtgagaagct

SEQ ID NO: 482 - DNA, 20 bp, synthetic construct
  source 1..20: mol_type = other DNA
gaccaatgtctgtgagaagc

SEQ ID NO: 483 - DNA, 20 bp, synthetic construct
  source 1..20: mol_type = other DNA
cgaccaatgtctgtgagaag

SEQ ID NO: 484 - DNA, 20 bp, synthetic construct
  source 1..20: mol_type = other DNA
gctgtcgtggtagacttaga

SEQ ID NO: 485 - DNA, 20 bp, synthetic construct
  source 1..20: mol_type = other DNA
ggctgtcgtggtagacttag

SEQ ID NO: 486 - DNA, 20 bp, synthetic construct
  source 1..20: mol_type = other DNA
gggctgtcgtggtagactta

SEQ ID NO: 487 - DNA, 20 bp, synthetic construct
  source 1..20: mol_type = other DNA
cgggctgtcgtggtagactt

SEQ ID NO: 488 - DNA, 20 bp, synthetic construct
  source 1..20: mol_type = other DNA
tgttggctgcattgtgccgg

SEQ ID NO: 489 - DNA, 20 bp, synthetic construct
  source 1..20: mol_type = other DNA
ttgttggctgcattgtgccg

SEQ ID NO: 490 - DNA, 20 bp, synthetic construct
  source 1..20: mol_type = other DNA
cttgttggctgcattgtgcc

SEQ ID NO: 491 - DNA, 20 bp, synthetic construct
  source 1..20: mol_type = other DNA
tcttgttggctgcattgtgc

SEQ ID NO: 492 - DNA, 20 bp, synthetic construct
  source 1..20: mol_type = other DNA
atcttgttggctgcattgtg

SEQ ID NO: 493 - DNA, 20 bp, synthetic construct
  source 1..20: mol_type = other DNA
aatcttgttggctgcattgt

SEQ ID NO: 494 - DNA, 20 bp, synthetic construct
  source 1..20: mol_type = other DNA
gaatcttgttggctgcattg

SEQ ID NO: 495 - DNA, 20 bp, synthetic construct
  source 1..20: mol_type = other DNA
agaatcttgttggctgcatt

SEQ ID NO: 496 - DNA, 20 bp, synthetic construct
  source 1..20: mol_type = other DNA
cagaatcttgttggctgcat

SEQ ID NO: 497 - DNA, 20 bp, synthetic construct
  source 1..20: mol_type = other DNA
gctgtgctcacacatgttct

SEQ ID NO: 498 - DNA, 20 bp, synthetic construct
  source 1..20: mol_type = other DNA
tgctgtgctcacacatgttc

SEQ ID NO: 499 - DNA, 20 bp, synthetic construct
  source 1..20: mol_type = other DNA
ttgctgtgctcacacatgtt

SEQ ID NO: 500 - DNA, 20 bp, synthetic construct
  source 1..20: mol_type = other DNA
gttgctgtgctcacacatgt

SEQ ID NO: 501 - DNA, 20 bp, synthetic construct
  source 1..20: mol_type = other DNA
tgttgctgtgctcacacatg

SEQ ID NO: 502 - DNA, 20 bp, synthetic construct
  source 1..20: mol_type = other DNA
gtgttgctgtgctcacacat

SEQ ID NO: 503 - DNA, 20 bp, synthetic construct
  source 1..20: mol_type = other DNA
ggtgttgctgtgctcacaca

SEQ ID NO: 504 - DNA, 20 bp, synthetic construct
  source 1..20: mol_type = other DNA
gggtgttgctgtgctcacac

SEQ ID NO: 505 - DNA, 20 bp, synthetic construct
  source 1..20: mol_type = other DNA
agggtgttgctgtgctcaca

SEQ ID NO: 506 - DNA, 20 bp, synthetic construct
  source 1..20: mol_type = other DNA
cagggtgttgctgtgctcac

SEQ ID NO: 507 - DNA, 20 bp, synthetic construct
  source 1..20: mol_type = other DNA
ccagggtgttgctgtgctca

SEQ ID NO: 508 - DNA, 20 bp, synthetic construct
  source 1..20: mol_type = other DNA
accagggtgttgctgtgctc

SEQ ID NO: 509 - DNA, 20 bp, synthetic construct
  source 1..20: mol_type = other DNA
gaccagggtgttgctgtgct

SEQ ID NO: 510 - DNA, 20 bp, synthetic construct
  source 1..20: mol_type = other DNA
tggaccagggtgttgctgtg

SEQ ID NO: 511 - DNA, 20 bp, synthetic construct
  source 1..20: mol_type = other DNA
ctggaccagggtgttgctgt

SEQ ID NO: 512 - DNA, 20 bp, synthetic construct
  source 1..20: mol_type = other DNA
tgctggaccagggtgttgct

SEQ ID NO: 513 - DNA, 20 bp, synthetic construct
  source 1..20: mol_type = other DNA
gtttccttcagagtctgggg

SEQ ID NO: 514 - DNA, 20 bp, synthetic construct
  source 1..20: mol_type = other DNA
tgtttccttcagagtctggg

SEQ ID NO: 515 - DNA, 20 bp, synthetic construct
  source 1..20: mol_type = other DNA
ccctgatttcatgtacttcc

SEQ ID NO: 516 - DNA, 20 bp, synthetic construct
  source 1..20: mol_type = other DNA
tccctgatttcatgtacttc

SEQ ID NO: 517 - DNA, 20 bp, synthetic construct
  source 1..20: mol_type = other DNA
cattccctgatttcatgtac

SEQ ID NO: 518 - DNA, 20 bp, synthetic construct
  source 1..20: mol_type = other DNA
cttgacattccctgatttca

SEQ ID NO: 519 - DNA, 20 bp, synthetic construct
  source 1..20: mol_type = other DNA
ccttgacattccctgatttc

SEQ ID NO: 520 - DNA, 20 bp, synthetic construct
  source 1..20: mol_type = other DNA
tccttgacattccctgattt

SEQ ID NO: 521 - DNA, 20 bp, synthetic construct
  source 1..20: mol_type = other DNA
gtccttgacattccctgatt

SEQ ID NO: 522 - DNA, 20 bp, synthetic construct
  source 1..20: mol_type = other DNA
ggtccttgacattccctgat

SEQ ID NO: 523 - DNA, 20 bp, synthetic construct
  source 1..20: mol_type = other DNA
aggtccttgacattccctga

SEQ ID NO: 524 - DNA, 20 bp, synthetic construct
  source 1..20: mol_type = other DNA
gaggtccttgacattccctg

SEQ ID NO: 525 - DNA, 20 bp, synthetic construct
  source 1..20: mol_type = other DNA
tgaggtccttgacattccct

SEQ ID NO: 526 - DNA, 20 bp, synthetic construct
  source 1..20: mol_type = other DNA
catatgttcctggcacagcc

SEQ ID NO: 527 - DNA, 20 bp, synthetic construct
  source 1..20: mol_type = other DNA
tcatatgttcctggcacagc

SEQ ID NO: 528 - DNA, 20 bp, synthetic construct
  source 1..20: mol_type = other DNA
gtcatatgttcctggcacag

SEQ ID NO: 529 - DNA, 20 bp, synthetic construct
  source 1..20: mol_type = other DNA
ggtcatatgttcctggcaca

SEQ ID NO: 530 - DNA, 20 bp, synthetic construct
  source 1..20: mol_type = other DNA
gggtcatatgttcctggcac

SEQ ID NO: 531 - DNA, 20 bp, synthetic construct
  source 1..20: mol_type = other DNA
ggggtcatatgttcctggca

SEQ ID NO: 532 - DNA, 20 bp, synthetic construct
  source 1..20: mol_type = other DNA
tggggtcatatgttcctggc

SEQ ID NO: 533 - DNA, 20 bp, synthetic construct
  source 1..20: mol_type = other DNA
ttggggtcatatgttcctgg

SEQ ID NO: 534 - DNA, 20 bp, synthetic construct
  source 1..20: mol_type = other DNA
gttggggtcatatgttcctg

SEQ ID NO: 535 - DNA, 20 bp, synthetic construct
  source 1..20: mol_type = other DNA
ggttggggtcatatgttcct

SEQ ID NO: 536 - DNA, 20 bp, synthetic construct
  source 1..20: mol_type = other DNA
tggttggggtcatatgttcc

SEQ ID NO: 537 - DNA, 20 bp, synthetic construct
  source 1..20: mol_type = other DNA
ctggttggggtcatatgttc

SEQ ID NO: 538 - DNA, 20 bp, synthetic construct
  source 1..20: mol_type = other DNA
gctggttggggtcatatgtt

SEQ ID NO: 539 - DNA, 20 bp, synthetic construct
  source 1..20: mol_type = other DNA
ggctggttggggtcatatgt

SEQ ID NO: 540 - DNA, 20 bp, synthetic construct
  source 1..20: mol_type = other DNA
gatgagggcgtgcagtgtgt

SEQ ID NO: 541 - DNA, 20 bp, synthetic construct
  source 1..20: mol_type = other DNA
tccctgtagtcccggatgag

SEQ ID NO: 542 - DNA, 20 bp, synthetic construct
  source 1..20: mol_type = other DNA
ctccctgtagtcccggatga

SEQ ID NO: 543 - DNA, 20 bp, synthetic construct
  source 1..20: mol_type = other DNA
tctccctgtagtcccggatg

SEQ ID NO: 544 - DNA, 20 bp, synthetic construct
  source 1..20: mol_type = other DNA
cttcttcttctccctgtagt

SEQ ID NO: 545 - DNA, 20 bp, synthetic construct
  source 1..20: mol_type = other DNA
tcttcttcttctccctgtag

SEQ ID NO: 546 - DNA, 20 bp, synthetic construct
  source 1..20: mol_type = other DNA
atcttcttcttctccctgta

SEQ ID NO: 547 - DNA, 20 bp, synthetic construct
  source 1..20: mol_type = other DNA
gatcttcttcttctccctgt

SEQ ID NO: 548 - DNA, 20 bp, synthetic construct
  source 1..20: mol_type = other DNA
ggatcttcttcttctccctg

SEQ ID NO: 549 - DNA, 20 bp, synthetic construct
  source 1..20: mol_type = other DNA
aggatcttcttcttctccct

SEQ ID NO: 550 - DNA, 20 bp, synthetic construct
  source 1..20: mol_type = other DNA
gttgagaaggatcttcttct

SEQ ID NO: 551 - DNA, 20 bp, synthetic construct
  source 1..20: mol_type = other DNA
tgttgagaaggatcttcttc

SEQ ID NO: 552 - DNA, 20 bp, synthetic construct
  source 1..20: mol_type = other DNA
gatgttgagaaggatcttct

SEQ ID NO: 553 - DNA, 20 bp, synthetic construct
  source 1..20: mol_type = other DNA
cgatgttgagaaggatcttc

SEQ ID NO: 554 - DNA, 20 bp, synthetic construct
  source 1..20: mol_type = other DNA
tcgatgttgagaaggatctt

SEQ ID NO: 555 - DNA, 20 bp, synthetic construct
  source 1..20: mol_type = other DNA
gatgcgatgctcgatgttga

SEQ ID NO: 556 - DNA, 20 bp, synthetic construct
  source 1..20: mol_type = other DNA
tgatgcgatgctcgatgttg

SEQ ID NO: 557 - DNA, 20 bp, synthetic construct
  source 1..20: mol_type = other DNA
atgatgcgatgctcgatgtt

SEQ ID NO: 558 - DNA, 20 bp, synthetic construct
  source 1..20: mol_type = other DNA
catgatgcgatgctcgatgt

SEQ ID NO: 559 - DNA, 20 bp, synthetic construct
  source 1..20: mol_type = other DNA
gttggatgggtgtctatctc

SEQ ID NO: 560 - DNA, 20 bp, synthetic construct
  source 1..20: mol_type = other DNA
ggttggatgggtgtctatct

SEQ ID NO: 561 - DNA, 20 bp, synthetic construct
  source 1..20: mol_type = other DNA
aggttggatgggtgtctatc

SEQ ID NO: 562 - DNA, 20 bp, synthetic construct
  source 1..20: mol_type = other DNA
caggttggatgggtgtctat

SEQ ID NO: 563 - DNA, 20 bp, synthetic construct
  source 1..20: mol_type = other DNA
tcaggttggatgggtgtcta

SEQ ID NO: 564 - DNA, 20 bp, synthetic construct
  source 1..20: mol_type = other DNA
atcaggttggatgggtgtct

SEQ ID NO: 565 - DNA, 20 bp, synthetic construct
  source 1..20: mol_type = other DNA
catcaggttggatgggtgtc

SEQ ID NO: 566 - DNA, 20 bp, synthetic construct
  source 1..20: mol_type = other DNA
gcatcaggttggatgggtgt

SEQ ID NO: 567 - DNA, 20 bp, synthetic construct
  source 1..20: mol_type = other DNA
tgtgcaggatcttcccactc

SEQ ID NO: 568 - DNA, 20 bp, synthetic construct
  source 1..20: mol_type = other DNA
atgtgcaggatcttcccact

SEQ ID NO: 569 - DNA, 20 bp, synthetic construct
  source 1..20: mol_type = other DNA
aatgtgcaggatcttcccac

SEQ ID NO: 570 - DNA, 20 bp, synthetic construct
  source 1..20: mol_type = other DNA
caatgtgcaggatcttccca

SEQ ID NO: 571 - DNA, 20 bp, synthetic construct
  source 1..20: mol_type = other DNA
tcaatgtgcaggatcttccc

SEQ ID NO: 572 - DNA, 20 bp, synthetic construct
  source 1..20: mol_type = other DNA
gtcaatgtgcaggatcttcc

SEQ ID NO: 573 - DNA, 20 bp, synthetic construct
  source 1..20: mol_type = other DNA
agtcaatgtgcaggatcttc

SEQ ID NO: 574 - DNA, 20 bp, synthetic construct
  source 1..20: mol_type = other DNA
aagtcaatgtgcaggatctt

SEQ ID NO: 575 - DNA, 20 bp, synthetic construct
  source 1..20: mol_type = other DNA
aaagtcaatgtgcaggatct

SEQ ID NO: 576 - DNA, 20 bp, synthetic construct
  source 1..20: mol_type = other DNA
caaagtcaatgtgcaggatc

SEQ ID NO: 577 - DNA, 20 bp, synthetic construct
  source 1..20: mol_type = other DNA
ccaaagtcaatgtgcaggat

SEQ ID NO: 578 - DNA, 20 bp, synthetic construct
  source 1..20: mol_type = other DNA
ttctgtagttgccatccagg

SEQ ID NO: 579 - DNA, 20 bp, synthetic construct
  source 1..20: mol_type = other DNA
attctgtagttgccatccag

SEQ ID NO: 580 - DNA, 20 bp, synthetic construct
  source 1..20: mol_type = other DNA
gattctgtagttgccatcca

SEQ ID NO: 581 - DNA, 20 bp, synthetic construct
  source 1..20: mol_type = other DNA
tgattctgtagttgccatcc

SEQ ID NO: 582 - DNA, 20 bp, synthetic construct
  source 1..20: mol_type = other DNA
gtgattctgtagttgccatc

SEQ ID NO: 583 - DNA, 20 bp, synthetic construct
  source 1..20: mol_type = other DNA
tgtgattctgtagttgccat

SEQ ID NO: 584 - DNA, 20 bp, synthetic construct
  source 1..20: mol_type = other DNA
atgtgattctgtagttgcca

SEQ ID NO: 585 - DNA, 20 bp, synthetic construct
  source 1..20: mol_type = other DNA
catgtgattctgtagttgcc

SEQ ID NO: 586 - DNA, 20 bp, synthetic construct
  source 1..20: mol_type = other DNA
gcatgtgattctgtagttgc

SEQ ID NO: 587 - DNA, 20 bp, synthetic construct
  source 1..20: mol_type = other DNA
ggcatgtgattctgtagttg

SEQ ID NO: 588 - DNA, 20 bp, synthetic construct
  source 1..20: mol_type = other DNA
tggcatgtgattctgtagtt

SEQ ID NO: 589 - DNA, 20 bp, synthetic construct
  source 1..20: mol_type = other DNA
gtggcatgtgattctgtagt

SEQ ID NO: 590 - DNA, 20 bp, synthetic construct
  source 1..20: mol_type = other DNA
tgtggcatgtgattctgtag

SEQ ID NO: 591 - DNA, 20 bp, synthetic construct
  source 1..20: mol_type = other DNA
gtgtggcatgtgattctgta

SEQ ID NO: 592 - DNA, 20 bp, synthetic construct
  source 1..20: mol_type = other DNA
tgtgtggcatgtgattctgt

SEQ ID NO: 593 - DNA, 20 bp, synthetic construct
  source 1..20: mol_type = other DNA
ctgtgtggcatgtgattctg

SEQ ID NO: 594 - DNA, 20 bp, synthetic construct
  source 1..20: mol_type = other DNA
actgtgtggcatgtgattct

SEQ ID NO: 595 - DNA, 20 bp, synthetic construct
  source 1..20: mol_type = other DNA
ttgcctttggtatttgtgtc

SEQ ID NO: 596 - DNA, 20 bp, synthetic construct
  source 1..20: mol_type = other DNA
gttgcctttggtatttgtgt

SEQ ID NO: 597 - DNA, 20 bp, synthetic construct
  source 1..20: mol_type = other DNA
tgttgcctttggtatttgtg

SEQ ID NO: 598 - DNA, 20 bp, synthetic construct
  source 1..20: mol_type = other DNA
cttgttgcctttggtatttg

SEQ ID NO: 599 - DNA, 20 bp, synthetic construct
  source 1..20: mol_type = other DNA
gcttgttgcctttggtattt

SEQ ID NO: 600 - DNA, 20 bp, synthetic construct
  source 1..20: mol_type = other DNA
cgcttgttgcctttggtatt

SEQ ID NO: 601 - DNA, 20 bp, synthetic construct
  source 1..20: mol_type = other DNA
tcgcttgttgcctttggtat

SEQ ID NO: 602 - DNA, 20 bp, synthetic construct
  source 1..20: mol_type = other DNA
atcgcttgttgcctttggta

SEQ ID NO: 603 - DNA, 20 bp, synthetic construct
  source 1..20: mol_type = other DNA
gatcgcttgttgcctttggt

SEQ ID NO: 604 - DNA, 20 bp, synthetic construct
  source 1..20: mol_type = other DNA
cgttcgggatcgcttgttgc

SEQ ID NO: 605 - DNA, 20 bp, synthetic construct
  source 1..20: mol_type = other DNA
tcgttcgggatcgcttgttg

SEQ ID NO: 606 - DNA, 20 bp, synthetic construct
  source 1..20: mol_type = other DNA
ctcgttcgggatcgcttgtt

SEQ ID NO: 607 - DNA, 20 bp, synthetic construct
  source 1..20: mol_type = other DNA
cctcgttcgggatcgcttgt

SEQ ID NO: 608 - DNA, 20 bp, synthetic construct
  source 1..20: mol_type = other DNA
tcctcgttcgggatcgcttg

SEQ ID NO: 609 - DNA, 20 bp, synthetic construct
  source 1..20: mol_type = other DNA
gtcctcgttcgggatcgctt

SEQ ID NO: 610 - DNA, 20 bp, synthetic construct
  source 1..20: mol_type = other DNA
gtaggaatccgtcctcgttc

SEQ ID NO: 611 - DNA, 20 bp, synthetic construct
  source 1..20: mol_type = other DNA
agtaggaatccgtcctcgtt

SEQ ID NO: 612 - DNA, 20 bp, synthetic construct
  source 1..20: mol_type = other DNA
gagtaggaatccgtcctcgt

SEQ ID NO: 613 - DNA, 20 bp, synthetic construct
  source 1..20: mol_type = other DNA
gtcgcttgtttgatgagcag

SEQ ID NO: 614 - DNA, 20 bp, synthetic construct
  source 1..20: mol_type = other DNA
tgtcgcttgtttgatgagca

SEQ ID NO: 615 - DNA, 20 bp, synthetic construct
  source 1..20: mol_type = other DNA
gtttcagtttagtggaagca

SEQ ID NO: 616 - DNA, 20 bp, synthetic construct
  source 1..20: mol_type = other DNA
ggtttcagtttagtggaagc

SEQ ID NO: 617 - DNA, 20 bp, synthetic construct
  source 1..20: mol_type = other DNA
tggtttcagtttagtggaag

SEQ ID NO: 618 - DNA, 20 bp, synthetic construct
  source 1..20: mol_type = other DNA
catggtttcagtttagtgga

SEQ ID NO: 619 - DNA, 20 bp, synthetic construct
  source 1..20: mol_type = other DNA
ccatggtttcagtttagtgg

SEQ ID NO: 620 - DNA, 20 bp, synthetic construct
  source 1..20: mol_type = other DNA
gtttcactgtcctgggaacc

SEQ ID NO: 621 - DNA, 20 bp, synthetic construct
  source 1..20: mol_type = other DNA
tgtttcactgtcctgggaac

SEQ ID NO: 622 - DNA, 20 bp, synthetic construct
  source 1..20: mol_type = other DNA
atgtttcactgtcctgggaa

SEQ ID NO: 623 - DNA, 20 bp, synthetic construct
  source 1..20: mol_type = other DNA
tatgtttcactgtcctggga

SEQ ID NO: 624 - DNA, 20 bp, synthetic construct
  source 1..20: mol_type = other DNA
ctatgtttcactgtcctggg

SEQ ID NO: 625 - DNA, 20 bp, synthetic construct
  source 1..20: mol_type = other DNA
actatgtttcactgtcctgg

SEQ ID NO: 626 - DNA, 20 bp, synthetic construct
  source 1..20: mol_type = other DNA
tactatgtttcactgtcctg

SEQ ID NO: 627 - DNA, 20 bp, synthetic construct
  source 1..20: mol_type = other DNA
gttatggatcttctgttccc

SEQ ID NO: 628 - DNA, 20 bp, synthetic construct
  source 1..20: mol_type = other DNA
agttatggatcttctgttcc

SEQ ID NO: 629 - DNA, 20 bp, synthetic construct
  source 1..20: mol_type = other DNA
gctgtgctgagtttgctgta

SEQ ID NO: 630 - DNA, 20 bp, synthetic construct
  source 1..20: mol_type = other DNA
ggctgtgctgagtttgctgt

SEQ ID NO: 631 - DNA, 20 bp, synthetic construct
  source 1..20: mol_type = other DNA
tggctgtgctgagtttgctg

SEQ ID NO: 632 - DNA, 20 bp, synthetic construct
  source 1..20: mol_type = other DNA
ttggctgtgctgagtttgct

SEQ ID NO: 633 - DNA, 20 bp, synthetic construct
  source 1..20: mol_type = other DNA
cttggctgtgctgagtttgc

SEQ ID NO: 634 - DNA, 20 bp, synthetic construct
  source 1..20: mol_type = other DNA
tcttggctgtgctgagtttg

SEQ ID NO: 635 - DNA, 20 bp, synthetic construct
  source 1..20: mol_type = other DNA
ttcttggctgtgctgagttt

SEQ ID NO: 636 - DNA, 20 bp, synthetic construct
  source 1..20: mol_type = other DNA
cctaatccatgttctccacg

SEQ ID NO: 637 - DNA, 20 bp, synthetic construct
  source 1..20: mol_type = other DNA
tcctaatccatgttctccac

SEQ ID NO: 638 - DNA, 20 bp, synthetic construct
  source 1..20: mol_type = other DNA
atcctaatccatgttctcca

SEQ ID NO: 639 - DNA, 20 bp, synthetic construct
  source 1..20: mol_type = other DNA
gatcctaatccatgttctcc

SEQ ID NO: 640 - DNA, 20 bp, synthetic construct
  source 1..20: mol_type = other DNA
ggatcctaatccatgttctc

SEQ ID NO: 641 - DNA, 20 bp, synthetic construct
  source 1..20: mol_type = other DNA
aggatcctaatccatgttct

SEQ ID NO: 642 - DNA, 20 bp, synthetic construct
  source 1..20: mol_type = other DNA
gtcagcatcttctgtgtctt

SEQ ID NO: 643 - DNA, 20 bp, synthetic construct
  source 1..20: mol_type = other DNA
ggtcagcatcttctgtgtct

SEQ ID NO: 644 - DNA, 20 bp, synthetic construct
  source 1..20: mol_type = other DNA
cttttgtactcattttggcc

SEQ ID NO: 645 - DNA, 20 bp, synthetic construct
  source 1..20: mol_type = other DNA
ccttttgtactcattttggc

SEQ ID NO: 646 - DNA, 20 bp, synthetic construct
  source 1..20: mol_type = other DNA
aattgaagcgtgtgagtcgc

SEQ ID NO: 647 - DNA, 20 bp, synthetic construct
  source 1..20: mol_type = other DNA
gaattgaagcgtgtgagtcg

SEQ ID NO: 648 - DNA, 20 bp, synthetic construct
  source 1..20: mol_type = other DNA
tgaattgaagcgtgtgagtc

SEQ ID NO: 649 - DNA, 20 bp, synthetic construct
  source 1..20: mol_type = other DNA
ttgaattgaagcgtgtgagt

SEQ ID NO: 650 - DNA, 20 bp, synthetic construct
  source 1..20: mol_type = other DNA
gtcttgaattgaagcgtgtg

SEQ ID NO: 651 - DNA, 20 bp, synthetic construct
  source 1..20: mol_type = other DNA
ggtcttgaattgaagcgtgt

SEQ ID NO: 652 - DNA, 20 bp, synthetic construct
  source 1..20: mol_type = other DNA
aggtcttgaattgaagcgtg

SEQ ID NO: 653 - DNA, 20 bp, synthetic construct
  source 1..20: mol_type = other DNA
caggtcttgaattgaagcgt

SEQ ID NO: 654 - DNA, 20 bp, synthetic construct
  source 1..20: mol_type = other DNA
gttgagtatttgttctgctc

SEQ ID NO: 655 - DNA, 20 bp, synthetic construct
  source 1..20: mol_type = other DNA
acatggtgtctagacatggc

SEQ ID NO: 656 - DNA, 20 bp, synthetic construct
  source 1..20: mol_type = other DNA
ctgatgtcatttattggcac

SEQ ID NO: 657 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
cacacctgatcaaatttctc

SEQ ID NO: 658 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
cgaggttttgttccgaagcc

SEQ ID NO: 659 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
ggggtaatgtgacgaggttt

SEQ ID NO: 660 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
ttaatattcgcgcggcttcg

SEQ ID NO: 661 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
tttcctttaatattcgcgcg

SEQ ID NO: 662 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
gtttcctttaatattcgcgc

SEQ ID NO: 663 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
ttcggctcacgctgggttgt

SEQ ID NO: 664 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
gggtaatgtgacgaggtttt

SEQ ID NO: 665 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
gcacacctgatcaaatttct

SEQ ID NO: 666 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
ctgctcagtcttcagaaaat

SEQ ID NO: 667 - DNA, 18 bp, synthetic construct
  source 1..18: mol_type = other DNA
cggcatgtctattttgta

SEQ ID NO: 668 - DNA, 20 bp, synthetic construct
  source 1..20: mol_type = other DNA
gttcgtttagagaacagatc

SEQ ID NO: 669 - DNA, 21 bp, synthetic construct
  source 1..21: mol_type = other DNA
taaagttcgtttagagaacag

SEQ ID NO: 670 - DNA, 15 bp, synthetic construct
  source 1..15: mol_type = other DNA
agccctgtatacgac

SEQ ID NO: 671 - DNA, 19 bp, synthetic construct
  source 1..19: mol_type = other DNA
gtaggtaaaaacctaatat

SEQ ID NO: 672 - DNA, 20 bp, synthetic construct
  source 1..20: mol_type = other DNA
cgtttagagaacagatctac

SEQ ID NO: 673 - DNA, 20 bp, synthetic construct
  source 1..20: mol_type = other DNA
cattgtagatgtcaaaagcc

SEQ ID NO: 674 - DNA, 21 bp, synthetic construct
  source 1..21: mol_type = other DNA
ctccctcatggtggcagttga

SEQ ID NO: 675 - DNA, 18 bp, synthetic construct
  source 1..18: mol_type = other DNA
tttgttcctggatgactc

SEQ ID NO: 676 - DNA, 18 bp, synthetic construct
  source 1..18: mol_type = other DNA
gtttgttcctggatgact

SEQ ID NO: 677 - DNA, 18 bp, synthetic construct
  source 1..18: mol_type = other DNA
agtttgttcctggatgac

SEQ ID NO: 678 - DNA, 18 bp, synthetic construct
  source 1..18: mol_type = other DNA
cagtttgttcctggatga

SEQ ID NO: 679 - DNA, 18 bp, synthetic construct
  source 1..18: mol_type = other DNA
tcagtttgttcctggatg

SEQ ID NO: 680 - DNA, 18 bp, synthetic construct
  source 1..18: mol_type = other DNA
ctcagtttgttcctggat

SEQ ID NO: 681 - DNA, 18 bp, synthetic construct
  source 1..18: mol_type = other DNA
tgtgtgtgtgtgcgtgtg

SEQ ID NO: 682 - DNA, 18 bp, synthetic construct
  source 1..18: mol_type = other DNA
gtgtgtgtgtgtgcgtgt

SEQ ID NO: 683 - DNA, 18 bp, synthetic construct
  source 1..18: mol_type = other DNA
tgtgtgtgtgtgtgcgtg

SEQ ID NO: 684 - DNA, 18 bp, synthetic construct
  source 1..18: mol_type = other DNA
gtgtgtgtgtgtgtgcgt

SEQ ID NO: 685 - DNA, 18 bp, synthetic construct
  source 1..18: mol_type = other DNA
tgtgtgtgtgtgtgtgcg

SEQ ID NO: 686 - DNA, 18 bp, synthetic construct
  source 1..18: mol_type = other DNA
gtgtgtgtgtgtgtgtgc

SEQ ID NO: 687 - DNA, 18 bp, synthetic construct
  source 1..18: mol_type = other DNA
tgtgtgtgtgtgtgtgtg

SEQ ID NO: 688 - DNA, 18 bp, synthetic construct
  source 1..18: mol_type = other DNA
gtgtgtgtgtgtgtgtgt

SEQ ID NO: 689 - DNA, 18 bp, synthetic construct
  source 1..18: mol_type = other DNA
tgtgtgtgtgtgtgtgtg

SEQ ID NO: 690 - DNA, 18 bp, synthetic construct
  source 1..18: mol_type = other DNA
gtgtgtgtgtgtgtgtgt

SEQ ID NO: 691 - DNA, 18 bp, synthetic construct
  source 1..18: mol_type = other DNA
tgtgtgtgtgtgtgtgtg

SEQ ID NO: 692 - DNA, 18 bp, synthetic construct
  source 1..18: mol_type = other DNA
gtgtgtgtgtgtgtgtgt

SEQ ID NO: 693 - DNA, 18 bp, synthetic construct
  source 1..18: mol_type = other DNA
tgtgtgtgtgtgtgtgtg

SEQ ID NO: 694 - DNA, 18 bp, synthetic construct
  source 1..18: mol_type = other DNA
gtgtgtgtgtgtgtgtgt

SEQ ID NO: 695 - DNA, 18 bp, synthetic construct
  source 1..18: mol_type = other DNA
cgtgtgtgtgtgtgtgtg

SEQ ID NO: 696 - DNA, 18 bp, synthetic construct
  source 1..18: mol_type = other DNA
gcgtgtgtgtgtgtgtgt

SEQ ID NO: 697 - DNA, 18 bp, synthetic construct
  source 1..18: mol_type = other DNA
tgcgtgtgtgtgtgtgtg

SEQ ID NO: 698 - DNA, 18 bp, synthetic construct
  source 1..18: mol_type = other DNA
gtgcgtgtgtgtgtgtgt

SEQ ID NO: 699 - DNA, 18 bp, synthetic construct
  source 1..18: mol_type = other DNA
agtggcggatctgaactc

SEQ ID NO: 700 - DNA, 18 bp, synthetic construct
  source 1..18: mol_type = other DNA
gagtggcggatctgaact

SEQ ID NO: 701 - DNA, 18 bp, synthetic construct
  source 1..18: mol_type = other DNA
gagtgtgctgcaggtaga

SEQ ID NO: 702 - DNA, 18 bp, synthetic construct
  source 1..18: mol_type = other DNA
tcgagtgtgctgcaggta

SEQ ID NO: 703 - DNA, 18 bp, synthetic construct
  source 1..18: mol_type = other DNA
atcgagtgtgctgcaggt

SEQ ID NO: 704 - DNA, 18 bp, synthetic construct
  source 1..18: mol_type = other DNA
gtgctgttgtagatggaa

SEQ ID NO: 705 - DNA, 18 bp, synthetic construct
  source 1..18: mol_type = other DNA
ggtgctgttgtagatgga

SEQ ID NO: 706 - DNA, 18 bp, synthetic construct
  source 1..18: mol_type = other DNA
tggtgctgttgtagatgg

SEQ ID NO: 707 - DNA, 18 bp, synthetic construct
  source 1..18: mol_type = other DNA
ctggtgctgttgtagatg

SEQ ID NO: 708 - DNA, 18 bp, synthetic construct
  source 1..18: mol_type = other DNA
cctggtgctgttgtagat

SEQ ID NO: 709 - DNA, 18 bp, synthetic construct
  source 1..18: mol_type = other DNA
ccctggtgctgttgtaga

SEQ ID NO: 710 - DNA, 18 bp, synthetic construct
  source 1..18: mol_type = other DNA
tccctggtgctgttgtag

SEQ ID NO: 711 - DNA, 18 bp, synthetic construct
  source 1..18: mol_type = other DNA
gtccctggtgctgttgta

SEQ ID NO: 712 - DNA, 18 bp, synthetic construct
  source 1..18: mol_type = other DNA
agtccctggtgctgttgt

SEQ ID NO: 713 - DNA, 18 bp, synthetic construct
  source 1..18: mol_type = other DNA
ctgggttggagatgttaa

SEQ ID NO: 714 - DNA, 18 bp, synthetic construct
  source 1..18: mol_type = other DNA
gctgggttggagatgtta

SEQ ID NO: 715 - DNA, 18 bp, synthetic construct
  source 1..18: mol_type = other DNA
cgctgggttggagatgtt

SEQ ID NO: 716 - DNA, 18 bp, synthetic construct
  source 1..18: mol_type = other DNA
tagcgctgggttggagat

SEQ ID NO: 717 - DNA, 18 bp, synthetic construct
  source 1..18: mol_type = other DNA
atgtagcgctgggttgga

SEQ ID NO: 718 - DNA, 18 bp, synthetic construct
  source 1..18: mol_type = other DNA
agccattcgccttctgct

SEQ ID NO: 719 - DNA, 18 bp, synthetic construct
  source 1..18: mol_type = other DNA
gtctttatggtgaagcca

SEQ ID NO: 720 - DNA, 18 bp, synthetic construct
  source 1..18: mol_type = other DNA
tgtctttatggtgaagcc

SEQ ID NO: 721 - DNA, 18 bp, synthetic construct
  source 1..18: mol_type = other DNA
ctgtctttatggtgaagc

SEQ ID NO: 722 - DNA, 18 bp, synthetic construct
  source 1..18: mol_type = other DNA
cctgtctttatggtgaag

SEQ ID NO: 723 - DNA, 18 bp, synthetic construct
  source 1..18: mol_type = other DNA
gttcctgtctttatggtg

SEQ ID NO: 724 - DNA, 18 bp, synthetic construct
  source 1..18: mol_type = other DNA
ggttcctgtctttatggt

SEQ ID NO: 725 - DNA, 18 bp, synthetic construct
  source 1..18: mol_type = other DNA
aggttcctgtctttatgg

SEQ ID NO: 726 - DNA, 18 bp, synthetic construct
  source 1..18: mol_type = other DNA
caggttcctgtctttatg

SEQ ID NO: 727 - DNA, 18 bp, synthetic construct
  source 1..18: mol_type = other DNA
ccaggttcctgtctttat

SEQ ID NO: 728 - DNA, 18 bp, synthetic construct
  source 1..18: mol_type = other DNA
ggtcttcccactgttttt

SEQ ID NO: 729 - DNA, 18 bp, synthetic construct
  source 1..18: mol_type = other DNA
aggagatgtggggtcttc

SEQ ID NO: 730 - DNA, 18 bp, synthetic construct
  source 1..18: mol_type = other DNA
caggagatgtggggtctt

SEQ ID NO: 731 - DNA, 18 bp, synthetic construct
  source 1..18: mol_type = other DNA
ggttggtctgttgtgact

SEQ ID NO: 732 - DNA, 18 bp, synthetic construct
  source 1..18: mol_type = other DNA
cggttggtctgttgtgac

SEQ ID NO: 733 - DNA, 18 bp, synthetic construct
  source 1..18: mol_type = other DNA
ccggttggtctgttgtga

SEQ ID NO: 734 - DNA, 18 bp, synthetic construct
  source 1..18: mol_type = other DNA
gagaatggttagaggttc

SEQ ID NO: 735 - DNA, 18 bp, synthetic construct
  source 1..18: mol_type = other DNA
gtagagaatggttagagg

SEQ ID NO: 736 - DNA, 18 bp, synthetic construct
  source 1..18: mol_type = other DNA
ggtgttttgccaatgtag

SEQ ID NO: 737 - DNA, 18 bp, synthetic construct
  source 1..18: mol_type = other DNA
gggtgttttgccaatgta

SEQ ID NO: 738 - DNA, 18 bp, synthetic construct
  source 1..18: mol_type = other DNA
tgggtgttttgccaatgt

SEQ ID NO: 739 - DNA, 18 bp, synthetic construct
  source 1..18: mol_type = other DNA
catcatcgttgtcgtcgt

SEQ ID NO: 740 - DNA, 18 bp, synthetic construct
  source 1..18: mol_type = other DNA
gaacggtacgtacagcaa

SEQ ID NO: 741 - DNA, 18 bp, synthetic construct
  source 1..18: mol_type = other DNA
ggaacggtacgtacagca

SEQ ID NO: 742 - DNA, 18 bp, synthetic construct
  source 1..18: mol_type = other DNA
aggaacggtacgtacagc

SEQ ID NO: 743 - DNA, 18 bp, synthetic construct
  source 1..18: mol_type = other DNA
taggaacggtacgtacag

SEQ ID NO: 744 - DNA, 18 bp, synthetic construct
  source 1..18: mol_type = other DNA
ataggaacggtacgtaca

SEQ ID NO: 745 - DNA, 18 bp, synthetic construct
  source 1..18: mol_type = other DNA
gataggaacggtacgtac

SEQ ID NO: 746 - DNA, 18 bp, synthetic construct
  source 1..18: mol_type = other DNA
ggataggaacggtacgta

SEQ ID NO: 747 - DNA, 18 bp, synthetic construct
  source 1..18: mol_type = other DNA
gggataggaacggtacgt

SEQ ID NO: 748 - DNA, 18 bp, synthetic construct
  source 1..18: mol_type = other DNA
gggtgcctattgcatagc

SEQ ID NO: 749 - DNA, 18 bp, synthetic construct
  source 1..18: mol_type = other DNA
agggtgcctattgcatag

SEQ ID NO: 750 - DNA, 18 bp, synthetic construct
  source 1..18: mol_type = other DNA
aagggtgcctattgcata

SEQ ID NO: 751 - DNA, 18 bp, synthetic construct
  source 1..18: mol_type = other DNA
gaagggtgcctattgcat

SEQ ID NO: 752 - DNA, 18 bp, synthetic construct
  source 1..18: mol_type = other DNA
ggaagggtgcctattgca

SEQ ID NO: 753 - DNA, 18 bp, synthetic construct
  source 1..18: mol_type = other DNA
tgggaagggtgcctattg

SEQ ID NO: 754 - DNA, 18 bp, synthetic construct
  source 1..18: mol_type = other DNA
atgggaagggtgcctatt

SEQ ID NO: 755 - DNA, 18 bp, synthetic construct
  source 1..18: mol_type = other DNA
aatgggaagggtgcctat

SEQ ID NO: 756 - DNA, 18 bp, synthetic construct
  source 1..18: mol_type = other DNA
gaatgggaagggtgccta

SEQ ID NO: 757 - DNA, 18 bp, synthetic construct
  source 1..18: mol_type = other DNA
agaatgggaagggtgcct

SEQ ID NO: 758 - DNA, 18 bp, synthetic construct
  source 1..18: mol_type = other DNA
aagaatgggaagggtgcc

SEQ ID NO: 759 - DNA, 18 bp, synthetic construct
  source 1..18: mol_type = other DNA
taagaatgggaagggtgc

SEQ ID NO: 760 - DNA, 18 bp, synthetic construct
  source 1..18: mol_type = other DNA
gtaagaatgggaagggtg

SEQ ID NO: 761 - DNA, 18 bp, synthetic construct
  source 1..18: mol_type = other DNA
agtaagaatgggaagggt

SEQ ID NO: 762 - DNA, 18 bp, synthetic construct
  source 1..18: mol_type = other DNA
gagtaagaatgggaaggg

SEQ ID NO: 763 - DNA, 18 bp, synthetic construct
  source 1..18: mol_type = other DNA
cagactttctcggtcata

SEQ ID NO: 764 - DNA, 18 bp, synthetic construct
  source 1..18: mol_type = other DNA
gcagactttctcggtcat

SEQ ID NO: 765 - DNA, 18 bp, synthetic construct
  source 1..18: mol_type = other DNA
tgcagactttctcggtca

SEQ ID NO: 766 - DNA, 18 bp, synthetic construct
  source 1..18: mol_type = other DNA
atgcagactttctcggtc

SEQ ID NO: 767 - DNA, 18 bp, synthetic construct
  source 1..18: mol_type = other DNA
aatgcagactttctcggt

SEQ ID NO: 768 - DNA, 18 bp, synthetic construct
  source 1..18: mol_type = other DNA
taatgcagactttctcgg

SEQ ID NO: 769 - DNA, 18 bp, synthetic construct
  source 1..18: mol_type = other DNA
gacctggacttttttccc

SEQ ID NO: 770 - DNA, 18 bp, synthetic construct
  source 1..18: mol_type = other DNA
tgacctggacttttttcc

SEQ ID NO: 771 - DNA, 18 bp, synthetic construct
  source 1..18: mol_type = other DNA
ctgacctggacttttttc

SEQ ID NO: 772 - DNA, 18 bp, synthetic construct
  source 1..18: mol_type = other DNA
gctgacctggactttttt

SEQ ID NO: 773 - DNA, 18 bp, synthetic construct
  source 1..18: mol_type = other DNA
ctgcaatgatgtggcaaa

SEQ ID NO: 774 - DNA, 18 bp, synthetic construct
  source 1..18: mol_type = other DNA
tctgcaatgatgtggcaa

SEQ ID NO: 775 - DNA, 18 bp, synthetic construct
  source 1..18: mol_type = other DNA
ttctgcaatgatgtggca

SEQ ID NO: 776 - DNA, 18 bp, synthetic construct
  source 1..18: mol_type = other DNA
cttctgcaatgatgtggc

SEQ ID NO: 777 - DNA, 18 bp, synthetic construct
  source 1..18: mol_type = other DNA
gctgcccacttgcatact

SEQ ID NO: 778 - DNA, 18 bp, synthetic construct
  source 1..18: mol_type = other DNA
gttggcagaacatagaac

SEQ ID NO: 779 - DNA, 18 bp, synthetic construct
  source 1..18: mol_type = other DNA
cgttggcagaacatagaa

SEQ ID NO: 780 - DNA, 18 bp, synthetic construct
  source 1..18: mol_type = other DNA
gcgttggcagaacataga

SEQ ID NO: 781 - DNA, 18 bp, synthetic construct
  source 1..18: mol_type = other DNA
atggggctacaggggata

SEQ ID NO: 782 - DNA, 18 bp, synthetic construct
  source 1..18: mol_type = other DNA
tatggggctacaggggat

SEQ ID NO: 783 - DNA, 18 bp, synthetic construct
  source 1..18: mol_type = other DNA
ttatggggctacagggga

SEQ ID NO: 784 - DNA, 18 bp, synthetic construct
  source 1..18: mol_type = other DNA
agttatggggctacaggg

SEQ ID NO: 785 - DNA, 18 bp, synthetic construct
  source 1..18: mol_type = other DNA
aagttatggggctacagg

SEQ ID NO: 786 - DNA, 18 bp, synthetic construct
  source 1..18: mol_type = other DNA
caagttatggggctacag

SEQ ID NO: 787 - DNA, 18 bp, synthetic construct
  source 1..18: mol_type = other DNA
ccaagttatggggctaca

SEQ ID NO: 788 - DNA, 18 bp, synthetic construct
  source 1..18: mol_type = other DNA
tccaagttatggggctac

SEQ ID NO: 789 - DNA, 18 bp, synthetic construct
  source 1..18: mol_type = other DNA
atccaagttatggggcta

SEQ ID NO: 790 - DNA, 18 bp, synthetic construct
  source 1..18: mol_type = other DNA
tatccaagttatggggct

SEQ ID NO: 791 - DNA, 18 bp, synthetic construct
  source 1..18: mol_type = other DNA
ctatccaagttatggggc

SEQ ID NO: 792 - DNA, 18 bp, synthetic construct
  source 1..18: mol_type = other DNA
attggaggaaatagggtg

SEQ ID NO: 793 - DNA, 18 bp, synthetic construct
  source 1..18: mol_type = other DNA
gtcttgtaggtagcagcc

SEQ ID NO: 794 - DNA, 18 bp, synthetic construct
  source 1..18: mol_type = other DNA
ggtcttgtaggtagcagc

SEQ ID NO: 795 - DNA, 18 bp, synthetic construct
  source 1..18: mol_type = other DNA
tggtcttgtaggtagcag

SEQ ID NO: 796 - DNA, 18 bp, synthetic construct
  source 1..18: mol_type = other DNA
ctggtcttgtaggtagca

SEQ ID NO: 797 - DNA, 18 bp, synthetic construct
  source 1..18: mol_type = other DNA
tctggtcttgtaggtagc

SEQ ID NO: 798 - DNA, 18 bp, synthetic construct
  source 1..18: mol_type = other DNA
gtctggtcttgtaggtag

SEQ ID NO: 799 - DNA, 18 bp, synthetic construct
  source 1..18: mol_type = other DNA
agtctggtcttgtaggta

SEQ ID NO: 800 - DNA, 18 bp, synthetic construct
  source 1..18: mol_type = other DNA
gagtctggtcttgtaggt

SEQ ID NO: 801 - DNA, 18 bp, synthetic construct
  source 1..18: mol_type = other DNA
ggagtctggtcttgtagg

SEQ ID NO: 802 - DNA, 18 bp, synthetic construct
  source 1..18: mol_type = other DNA
aggagtctggtcttgtag

SEQ ID NO: 803 - DNA, 18 bp, synthetic construct
  source 1..18: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
cggcatgtctattttgta

SEQ ID NO: 804 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
gttcgtttagagaacagatc

SEQ ID NO: 805 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
taaagttcgtttagagaacag

SEQ ID NO: 806 - DNA, 15 bp, synthetic construct
  source 1..15: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
agccctgtatacgac

SEQ ID NO: 807 - DNA, 19 bp, synthetic construct
  source 1..19: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
gtaggtaaaaacctaatat

SEQ ID NO: 808 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
cgtttagagaacagatctac

SEQ ID NO: 809 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
cattgtagatgtcaaaagcc

SEQ ID NO: 810 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
ctccctcatggtggcagttga

(19) United States
(12) Patent Application Publication
(10) Pub. No.: US 2026/0265745 A1
(43) Pub. Date: Sep. 10, 2026

(54) TGFB2-IRF5 THERAPEUTICS FOR CANCER

(71) Applicant: GMP BIOTECHNOLOGY LIMITED, Hong Kong (CN)

(72) Inventor: Vuong TRIEU, Agoura Hills, CA (US)

(21) Appl. No.: 19/403,128

(22) Filed: Nov. 27, 2025

Related U.S. Application Data
(63) Continuation of application No. PCT/US2024/032478, filed on Jun. 5, 2024.
(60) Provisional application No. 63/471,168, filed on Jun. 5, 2023.
(60) Provisional application No. 63/535,431, filed on Aug. 30, 2023.
(60) Provisional application No. 63/567,406, filed on Mar. 19, 2024.

(51) Int. Cl. C12N 15/113; A61K 31/277; A61K 31/282; A61K 31/337; A61K 31/436; A61K 31/4745; A61K 31/495; A61K 31/506; A61K 31/513; A61K 31/517; A61K 31/519; A61K 31/5377; A61K 31/7068; A61K 39/395; A61P 35/00

(52) U.S. Cl. CPC C12N 15/113; A61K 31/277; A61K 31/282; A61K 31/337; A61K 31/436; A61K 31/4745; A61K 31/495; A61K 31/506; A61K 31/513; A61K 31/517; A61K 31/519; A61K 31/5377; A61K 31/7068; A61K 39/3955; A61P 35/00; C12N 2310/11; C12N 2310/351; C12N 2310/3515

(57) ABSTRACT

This invention relates to agents, uses, and methods for treating cancer with an agent for suppressing expression of IRF5. Synergistic therapies include agents, uses, and methods for treating cancer with an agent for suppressing expression of IRF5 in combination with agents for suppressing expression of TGF-β2. One or more biomarkers can be used to select subjects who benefit from the methods, agents or uses, including IFNGR2, JAK1, and STAT1, as well as TGF-β2 and one or more of IRF5, TLR9, FOXP3, CCL22, CREB5, CD8a, CD86, CC14, CD163, ITGAX, and CD11c. The agents and compositions can be used in combination with chemotherapy and other standard of care therapies.

### SEQUENCE LISTING

This application includes a sequence listing submitted electronically as an ST.26 file created on Nov. 25, 2025, named 018988-015US1_SL.xml, which is 904,128 bytes in size.

### TECHNICAL FIELD

This invention relates to agents, uses, and methods for treating cancer with an agent for suppressing expression of IRF5. Synergistic therapies further include agents, uses, and methods for treating cancer with an agent for suppressing expression of IRF5 in combination with agents for suppressing expression of TGF-β2. One or more biomarkers can be used to select subjects who benefit from the methods, agents or uses, including IFNGR2, JAK1, and STAT1, as well as TGF-β2 and one or more of IRF5, TLR9, FOXP3, CCL22, CREB5, CD8a, CD86, CC14, CD163, ITGAX, and CD11c. The agents and compositions can be used in combination with chemotherapy and other standard-of-care therapies.

### BACKGROUND

Cancer is a complex pathology involving multiple variant cellular pathways. Because of this complexity, many anti-cancer drugs have limited or partial therapeutic effectiveness.

Drawbacks of conventional therapies include lack of efficacy as determined by overall survival.

Further drawbacks of conventional therapies include significant unwanted side effects such as killing healthy cells in addition to killing cancer cells.

Additional drawbacks of anti-cancer agents include high toxicity at required levels of therapeutic administration.

What is needed are methods, agents and uses for cancer diseases to increase efficacy, and reduce toxicity and unwanted side effects.

Therapeutic compositions of different agents are needed to supply significant anti-tumor effects and cancer immunotherapeutic effects and which can reduce side effects and adverse health effects. There is an urgent need for improved guidance for use of such compositions by using appropriate biomarkers to select synergistic effects of the compositions.

### BRIEF SUMMARY

This invention relates to agents, uses, and methods for treating cancer with an agent for inhibiting or suppressing expression of IRF5, alone or in combination with other agents.

Embodiments of this invention provide synergistic therapies which include agents, uses, and methods for treating cancer with an agent for inhibiting or suppressing expression of IRF5 in combination with agents for inhibiting or suppressing expression of TGF-β2.

This disclosure further encompasses use of biomarkers to select subjects who benefit from the methods, agents or uses disclosed herein, including IFNGR2, JAK1, and STAT1, as well as TGF-β2 and one or more of IRF5, TLR9, FOXP3, CCL22, CREB5, CD8a, CD86, CC14, CD163, ITGAX, and CD11c.

Anti-cancer agents and compositions of this disclosure can also be used in combination with chemotherapy and other standard of care therapies for cancer.

In some embodiments, methods and therapeutic strategies of this invention can increase efficacy, as well as reduce toxic side effects and adverse health effects in cancer treatment.

In further embodiments, methods and therapeutic strategies of this invention can improve guidance of the therapy using appropriate biomarkers to select synergistic effects of the compositions.

Embodiments of this invention include the following:

An agent for inhibiting or suppressing expression of IRF5 for treating or ameliorating the symptoms of cancer in a subject.

Use of a composition comprising an agent for inhibiting or suppressing expression of IRF5 in the preparation of a medicament for treating or ameliorating the symptoms of cancer in a subject.

A method for treating or ameliorating the symptoms of cancer in a subject in need, the method comprising: preparing a pharmaceutical composition comprising an agent for inhibiting or suppressing expression of IRF5; and administering a therapeutically sufficient amount of the composition to the subject.

The agent, use or method above, wherein the cancer is a glioma, low grade glioma, glioblastoma, diffuse intrinsic pontine glioma (DIPG), diffuse midline glioma (DMG), leptomeningeal or brain metastasis, brain or spinal cancer, or CNS tumors.

The agent, use or method above, wherein the cancer is a pancreatic cancer.

The agent, use or method above, comprising using one or more biomarkers to select the subjects who benefit from the agent, use or method, wherein the biomarkers are an elevated level of TGF-β2 and an elevated level of one or more of IFNGR2, JAK1, and STAT1.

The agent, use or method above, comprising using one or more biomarkers to select the subjects who benefit from the agent, use or method, wherein the biomarkers are levels of TGF-β2 and one or more of IRF5, TLR9, FOXP3, CCL22, CREB5, CD8a, CD86, CC14, CD163, ITGAX, and CD11c.

The agent, use or method above, wherein the agent, medicament or administration comprises one or more IRF5-specific antisense oligonucleotides complementary to a IRF5 transcript and 15-30 nucleotides in length.

The agent, use or method above, wherein the agent, medicament or administration comprises one or more IRF5-specific antisense oligonucleotides complementary to a IRF5 pre-RNA, pre-mRNA or mRNA and 18-21 nucleotides in length.

The agent, use or method above, wherein the agent, medicament or administration comprises one or more IRF5-specific antisense oligonucleotides complementary to a IRF5 transcript as in any of Table 1, Table 2, and Table 3.

The agent, use or method above, wherein the agent, medicament or administration comprises IRF5-specific antisense oligonucleotides CACACCTGATCAAATTTCTC SEQ ID NO: 1 or C*A*C*A*C*C*T*G*A*T*C*A*A*A*T*T*T*C*T*C SEQ ID NO:657.

The agent, use or method above, comprising a IRF5-specific antisense oligonucleotide as in any of Table 1, Table 2, and Table 3 having one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the agent is conjugated to a polyethylene glycol, a lipid, or a triantenarry N-acteyl-galactosamine.

The agent, use or method above, comprising a carrier of sterile water for injection, saline, isotonic saline, phosphate buffered saline, or a combination thereof.

The agent, use or method above, wherein the agent, medicament or administration is substantially free of excipients.

The agent, use or method above, wherein the agent, medicament or administration is stable for at least 14 days in carrier at 37° C.

The agent, use or method above, wherein the agent, medicament or administration is combined with a standard of care treatment for the cancer.

The agent, use or method above, wherein the agent is administered by infusion, injection, or intracranial continuous infusion.

The agent, use or method above, comprising any one or more additional medicaments comprising a targeted cancer drug, a cancer growth blocker, or an EGFR inhibitor, erlotinib, gefitinib, afatinib, osimertinib, dacomitininb, and combinations thereof.

The agent, use or method above, comprising any one or more additional medicaments which are targeted cancer drugs selected from bevacizumab, everolimus, belzutifan, dabrafenib, trametinib, and combinations thereof.

The agent, use or method above, comprising any one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, comprising any one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

The agent, use or method above, wherein the agent, medicament or administration decreases mortality rate of subjects at month 6, 12, 18, 24, 30, or 36.

The agent, use or method above, wherein the agent, medicament or administration increases survival rate of subjects at month 6, 12, 18, 24, 30, or 36.

A kit comprising: an agent described above and a carrier.

An agent for inhibiting or suppressing expression of IRF5 in combination with an agent for inhibiting or suppressing expression of TGF-β2 for treating or ameliorating the symptoms of cancer in a subject.

Use of a composition comprising an agent for inhibiting or suppressing expression of IRF5 in combination with an agent for inhibiting or suppressing expression of TGF-β2 in the preparation of a medicament for treating or ameliorating the symptoms of cancer in a subject.

A method for treating or ameliorating the symptoms of cancer in a subject in need, the method comprising: preparing a pharmaceutical composition comprising an agent for inhibiting or suppressing expression of IRF5 in combination with an agent for inhibiting or suppressing expression of TGF-β2; and administering a therapeutically sufficient amount of the composition to the subject.

The agent, use or method above, wherein the cancer is a glioma, low grade glioma, glioblastoma, diffuse intrinsic pontine glioma (DIPG), diffuse midline glioma (DMG), leptomeningeal or brain metastasis, brain or spinal cancer, or CNS tumors.

The agent, use or method above, wherein the cancer is a pancreatic cancer.

The agent, use or method above, comprising using one or more biomarkers to select subjects who benefit from the agent, use or method, wherein the biomarkers are levels of TGF-β2 and one or more of IFNGR2, STAT1, IRF1, IRF5, CD276, and CD204.

The agent, use or method above, wherein the cancer is low grade glioma having tumor cells exhibiting wild type IDH1 or IDH2 and one or more of upregulated IFNGR2, upregulated STAT1, upregulated IRF1, upregulated IRF5, upregulated CD276, and upregulated CD204.

The agent, use or method above, wherein the IRF5 agent, medicament or administration comprises one or more IRF5-specific antisense oligonucleotides complementary to a IRF5 transcript and 15-30 nucleotides in length.

The agent, use or method above, wherein the IRF5 agent, medicament or administration comprises one or more IRF5-specific antisense oligonucleotides complementary to a IRF5 pre-RNA, pre-mRNA or mRNA and 18-21 nucleotides in length.

The agent, use or method above, wherein the IRF5 agent, medicament or administration comprises one or more IRF5-specific antisense oligonucleotides complementary to a IRF5 transcript as in any of Table 1, Table 2, and Table 3.

The agent, use or method above, wherein the agent for inhibiting or suppressing expression of IRF5 is YE6144 (S,E)-N1-(6-Fluoro-3-(2-(6-morpholinopyridazin-3-yl) vinyl)-1H-indazol-5-yl) butane-1,2-diamine Hydrochloride, a cell-penetrating peptide inhibitor of IRF5 dimerization, an NLS peptide mimic, or a decoy peptide.

The agent, use or method above, wherein the TGF-β2 agent, medicament or administration comprises one or more TGF-β2-specific antisense oligonucleotides complementary to a TGF-β2 transcript and 15-30 nucleotides in length.

The agent, use or method above, wherein the TGF-β2 agent, medicament or administration comprises one or more TGF-β2-specific antisense oligonucleotides complementary to a TGF-β2 pre-RNA, pre-mRNA or mRNA and 18-21 nucleotides in length. The agent, use or method above, wherein the TGF-β2 agent, medicament or

administration comprises one or more TGF-β2-specific antisense oligonucleotides complementary to a TGF-β2 transcript as in any of Table 4 and Table 5.

The agent, use or method above, wherein the agent, medicament or administration comprises the combination CACACCTGATCAAATTTCTC SEQ ID NO: 1 and CGGCATGTCTATTTTGTA SEQ ID NO:667, or C*A*C*A*C*C*T*G*A*T*C*A*A*A*T*T*T*C*T*C SEQ ID NO:657 and C*G*G*C*A*T*G*T*C*T*A*T*T*T*T*G*T*A SEQ ID NO:803.

The agent, use or method above, wherein the antisense oligonucleotides are as in any of Table 1, Table 2, Table 3, Table 4 and Table 5 and comprise one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the agent is conjugated to a polyethylene glycol, a lipid, or a triantenarry N-acteyl-galactosamine.

The agent, use or method above, comprising a carrier of sterile water for injection, saline, isotonic saline, phosphate buffered saline, or a combination thereof.

The agent, use or method above, wherein the agent, medicament or administration is substantially free of excipients.

The agent, use or method above, wherein the agent, medicament or administration is stable for at least 14 days in carrier at 37° C.

The agent, use or method above, wherein the agent, medicament or administration is combined with a standard of care treatment for the cancer.

The agent, use or method above, wherein the agent for inhibiting or suppressing expression of IRF5 and the agent for inhibiting or suppressing expression of TGF-β2 are administered concurrently, simultaneously, sequentially, or separately in time.

The agent, use or method above, wherein the agents are administered by infusion, injection, or intracranial continuous infusion.

The agent, use or method above, comprising any one or more additional medicaments comprising a targeted cancer drug, a cancer growth blocker, or an EGFR inhibitor, erlotinib, gefitinib, afatinib, osimertinib, dacomitininb, and combinations thereof.

The agent, use or method above, comprising any one or more additional medicaments which are targeted cancer drugs selected from bevacizumab, everolimus, belzutifan, dabrafenib, trametinib, and combinations thereof.

The agent, use or method above, comprising any one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, comprising any one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

The agent, use or method above, wherein the agent, medicament or administration decreases mortality rate of subjects at month 6, 12, 18, 24, 30, or 36.

The agent, use or method above, wherein the agent, medicament or administration increases survival rate of subjects at month 6, 12, 18, 24, 30, or 36.

A kit comprising: an agent described above and a carrier.

### BRIEF DESCRIPTION OF THE DRAWINGS

FIG. 1 shows results of a study of clinical outcomes for pancreatic cancer patients (PDAC) and the beneficial impact on overall survival for pancreatic cancer patients of the therapeutic use of an IRF5-specific antisense agent in combination with a TGF-β2-specific antisense agent. The Kaplan-Meier chart of FIG. 1 (KM Plotter) shows that overall survival was significantly improved for patients below median IRF5 and having low TGF-β2. This study established a basis for therapeutic use of an IRF5-specific antisense agent in combination with a TGF-2-specific antisense agent for treating pancreatic cancer. The median overall survival time for patients from the IRF5 (low)-TGFβ2 (low) group was 38 months (logrank P=0.00059), which was significantly and surprisingly increased over the 16 months observed for patients of the IRF5 (low)-TGFβ2 (high) group.

FIG. 2 shows results of a study of clinical outcomes for 513 low grade glioma patients (cBioPortal) and the beneficial impact on overall survival for low grade glioma patients of the therapeutic use of an IRF5-specific antisense agent. The Kaplan-Meier chart of FIG. 2 shows that overall survival was significantly improved for patients below median IRF5. This study established a basis for therapeutic use of an IRF5-specific antisense agent for treating low grade glioma. The median overall survival time for patients from the IRF5 (low) group was 95 months (logrank P<0.0001), which was significantly and surprisingly increased over the 64 months observed for patients of the IRF5 (high) group.

FIG. 3 shows results of a study of clinical outcomes for 513 low grade glioma patients (cBioPortal) and the beneficial impact on overall survival for low grade glioma patients of the therapeutic use of an IRF5-specific antisense agent in combination with a TGF-β2-specific antisense agent. The Kaplan-Meier chart of FIG. 3 shows that overall survival was significantly improved for patients below median IRF5 having low TGF-β2. This study established a basis for therapeutic use of an IRF5-specific antisense agent in combination with a TGF-β2-specific antisense agent for treating low grade glioma. The median overall survival time for patients from the IRF5 (low)-TGFβ2 (low) group was 105 months (logrank P<0.0001), which was significantly and surprisingly increased over the 27 months observed for patients of the IRF5 (high)-TGFβ2 (high) group.

FIG. 4 shows results of a study of pediatric DIPG tumor samples. mRNA expression levels were obtained for IFNGR2 (N=45), JAK1 (N=45), and STAT1 (N=45), represented as log 2-transformed transcripts per million (TPM), for primary tumor samples. IFNGR2 mRNA levels were significantly upregulated in DIPG samples as compared to normal pons tissue (1.58-fold increase; P=0.0006). The pediatric DIPG patients whose brain tumors were localized to pons/brainstem included molecular subtype classifications DMG, H3K27M (N=23); DMG, H3K27M, TP53 (N=8); HGG, H3 wildtype (N=2); HGG, H3 wildtype, TP53 (N=1); HGG, to be classified (N=10) and 1 not determined. The mRNA expression levels in DIPG samples were compared to levels in normal pons samples from 29 pons regions (21 subjects). The bar charts illustrate mean expression levels for mRNA in tumor specimens (dark grey bars) as compared to normal pons samples (light grey bars). The statistical significance of differences in mRNA expression levels was assessed using two-way ANOVA.

FIG. 5 shows results of a study of pediatric DIPG tumor samples. Antigen-presenting cell mRNA expression levels in pediatric DIPG tumors were downregulated as compared to normal brainstem tissue. CD14 (N=45), CD163 (N=45), CD86 (N=45), and ITGAX (N=45) mRNA expression levels were obtained (log 2 TPM) in pediatric DIPG samples and compared to the expression in normal pons samples. The bar charts illustrate mean expression levels for mRNA in tumor specimens from pediatric DIPG patients (dark grey bars) compared to normal pons samples (light grey bars). Compared to normal brainstem/pons tissue, CD14, CD163, and ITGAX mRNA expression in pediatric DIPG patients was significantly decreased by 1.64-fold (P=0.037), 1.75-fold (P=0.019), and 3.33-fold (P<0.0001), respectively. Differences in mRNA expression levels were assessed using two-way ANOVA.

### DETAILED DESCRIPTION OF THE DISCLOSURE

This invention relates to methods, compositions and uses thereof for treating or ameliorating the symptoms of cancer in a human or animal subject with pharmaceutical compositions designed to promote anti-tumor effects.

Embodiments of this invention contemplate suppressing expression of IRF5 mRNA, for example with antisense oligonucleotides.

This invention relates to agents, compositions, uses, drug products and methods for treating cancer with agents for inhibiting or suppressing expression of IRF5, agents for inhibiting or suppressing expression of TGF-β2, and a combination thereof. These agents may be used in combination with a checkpoint inhibitor.

Synergistic therapies include combinations of agents for inhibiting or suppressing IRF5, alone or with agents for inhibiting or suppressing expression of TGF-β2.

In certain embodiments, an agent for suppressing expression of IRF5 can be used alone or in combination with an agent for suppressing expression of TGF-β2 for treating or ameliorating the symptoms of cancer in a subject.

Further embodiments include use of a composition comprising an agent for suppressing expression of IRF5 alone or in combination with an agent for suppressing expression of TGF-β2 in the preparation of a medicament for treating or ameliorating the symptoms of cancer in a subject.

Additional embodiments include methods for treating or ameliorating the symptoms of cancer in a subject in need, the method comprising: preparing a pharmaceutical composition comprising an agent for suppressing expression of IRF5 alone or in combination with an agent for suppressing expression of TGF-β2; and administering the composition to the subject.

Without wishing to be bound by theory, Applicants have discovered that of the three isoforms of TGF-β, only TGF-β2 was found to be associated with cancer pathology. Thus, embodiments of this invention provide agents and methods for inhibiting or suppressing expression of TGF-β2, in combination with other agents, which provides surprising improvement of overall survival in cancer patients.

In some embodiments, one or more biomarkers can be used to select subjects who benefit from the method, agent or use, including IRF5 and/or ITGAM. Additional biomarkers include IFNGR2, JAKI, and STAT1, as well as IRF5, TLR9, FOXP3, CCL22, CREB5, CD8a, CD86, IFNGR2, CC14, CD163, ITGAX, and CD11c.

As used herein, the term agent can refer to one or more active compounds, a combination of active compounds, or a composition containing one or more active compounds and a carrier, and/or a solvent, and/or any number of excipients. In some embodiments, the composition may be a pharmaceutical composition. In certain embodiments, the composition may be a pharmaceutical composition containing a therapeutically effective amount of one or more active compounds. Some examples of excipients are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980. Methods for determining a therapeutically effective amount of a compound are known in the art.

In some embodiments, methods and therapeutic strategies of this invention can increase efficacy, as well as reduce toxic side effects and adverse health effects in cancer treatment.

In further embodiments, methods and therapeutic strategies of this invention can improve guidance of the therapy using appropriate biomarkers to select synergistic effects of the compositions.

### Human IRF5-Specific Antisense Oligodeoxynucleotides

Embodiments of this invention further include pharmaceutical compositions for inhibiting or suppressing expression of IRF5, or for treating or ameliorating the symptoms of cancer in a human or animal. The pharmaceutical compositions may contain a pharmaceutically acceptable salt form, an ester form, or a polymorph or stereoisomer of any active agent of this disclosure, as well as a carrier. The IRF5 inhibitor may be selected from IRF5-specific antisense oligonucleotides. The carrier may be sterile water for injection, saline, isotonic saline, or a combination thereof.

IRF5 antisense may be chemically-modified in the same manner as described below for TGF-beta-2 antisense.

Examples of agents of this disclosure for inhibiting or suppressing expression of IRF5 include IRF5-specific antisense oligonucleotides given in Table 1.

TABLE 1
IRF5-specific antisense oligonucleotides

| SEQ ID | | ANTISENSE SEQUENCE |
|---|---|---|
| NO: | Ref. | (5-3) |
| 1 | 1250 1269 | CACACCTGATCAAATTTCTC |
| 2 | 1061 1080 | CGAGGTTTTGTTCCGAAGCC |
| 3 | 1073 1092 | GGGGTAATGTGACGAGGTTT |
| 4 | 96 115 | TTAATATTCGCGCGGCTTCG |
| 5 | 102 121 | TTTCCTTTAATATTCGCGCG |
| 6 | 103 122 | GTTTCCTTTAATATTCGCGC |
| 7 | 806 825 | TTCGGCTCACGCTGGGTTGT |
| 8 | 1072 1091 | GGGTAATGTGACGAGGTTTT |
| 9 | 1251 1270 | GCACACCTGATCAAATTTCT |
| 10 | 2757 2776 | CTGCTCAGTCTTCAGAAAAT |
| 11 | 4207 4226 | AGTTTAAGAGGGTCTGTGTG |
| 12 | 4208 4227 | AAGTTTAAGAGGGTCTGTGT |
| 13 | 5202 5221 | TGTTGGCAGAGGATGGTCAA |
| 14 | 5204 5223 | ACTGTTGGCAGAGGATGGTC |
| 15 | 5554 5573 | GGTTCTGATGTTTGTAGTGT |
| 16 | 5555 5574 | TGGTTCTGATGTTTGTAGTG |
| 17 | 5560 5579 | GGGCTTGGTTCTGATGTTTG |
| 18 | 5561 5580 | CGGGCTTGGTTCTGATGTTT |
| 19 | 6568 6587 | TGGGGTCATATGTTCCTGGC |
| 20 | 6569 6588 | TTGGGGTCATATGTTCCTGG |

In some embodiments, the following criteria can be used for an antisense oligonucleotide:

$$\begin{matrix}A ) & \\ 40 \% <= GC \% <= 60 \% ; & \end{matrix}$$

B) No GGGG in the target sequence;
C) Average unpaired probability for target site nucleotides>=0.5;
D) For each peak in the accessibility profile that can be above the threshold probability of 0.5, all sites targeted to this same peak may be ranked by their average unpaired probability (the higher the better) and at most n sites can be selected for each peak, where n is determined by max ([width of peak/site length], 2);
E) Among sites satisfying criteria A-D, the top 20 unique ones with the highest average unpaired probability may be listed.

In certain embodiments, the average unpaired probability can be used in filter criteria C, D and E to cut down the number of reported sites in order to make the disruption energy calculation as manageable.

The IRF5 antisense sequences can be chemically-modified to provide active variants thereof, LNA variants thereof, as well as gapmer variants thereof, as known in the art. The sequences can be used in any combination as active agents, such as pooling combinations.

In some embodiments, the IRF5 antisense sequences can be n-M-n RNA (2′-OMe)*-DNA*-RNA (2′-OMe)* gapmers, where n is from 3-7 and M is from 6-12. In certain embodiments, the IRF5 antisense sequences can be 3-10-3 or 5-10-5 LNA*-DNA*-LNA* or cEt*-DNA*-cEt* gapmers.

It is understood that additional antisense oligonucleotides in various formats can be constructed based on the IRF5 gene sequence.

Additional examples of agents of this disclosure for inhibiting or suppressing expression of IRF5 include IRF5-specific antisense oligonucleotides given in Table 2.

TABLE 2
IRF5-specific antisense oligonucleotides

| SEQ ID | | ANTISENSE SEQUENCE |
|---|---|---|
| NO: | Ref. | (5-3) |
| 21. | 94-113 | AATATTCGCGCGGCTTCGGC |
| 22. | 95-114 | TAATATTCGCGCGGCTTCGG |
| 23. | 96-115 | TTAATATTCGCGCGGCTTCG |
| 24. | 97-116 | TTTAATATTCGCGCGGCTTC |
| 25. | 98-117 | CTTTAATATTCGCGCGGCTT |
| 26. | 100-119 | TCCTTTAATATTCGCGCGGC |
| 27. | 101-120 | TTCCTTTAATATTCGCGCGG |
| 28. | 102-121 | TTTCCTTTAATATTCGCGCG |
| 29. | 103-122 | GTTTCCTTTAATATTCGCGC |
| 30. | 141-160 | GGTTCCAAGCATCTTGCCCT |
| 31. | 142-161 | CGGTTCCAAGCATCTTGCCC |
| 32. | 143-162 | CCGGTTCCAAGCATCTTGCC |
| 33. | 144-163 | TCCGGTTCCAAGCATCTTGC |
| 34. | 145-164 | GTCCGGTTCCAAGCATCTTG |
| 35. | 146-165 | GGTCCGGTTCCAAGCATCTT |
| 36. | 147-166 | AGGTCCGGTTCCAAGCATCT |
| 37. | 148-167 | CAGGTCCGGTTCCAAGCATC |
| 38. | 149-168 | GCAGGTCCGGTTCCAAGCAT |
| 39. | 230-249 | TCATTCCGGCTCTTTAGGCC |
| 40. | 231-250 | CTCATTCCGGCTCTTTAGGC |
| 41. | 232-251 | CCTCATTCCGGCTCTTTAGG |
| 42. | 233-252 | TCCTCATTCCGGCTCTTTAG |
| 43. | 234-253 | TTCCTCATTCCGGCTCTTTA |
| 44. | 235-254 | TTTCCTCATTCCGGCTCTTT |
| 45. | 236-255 | GTTTCCTCATTCCGGCTCTT |
| 46. | 237-256 | GGTTTCCTCATTCCGGCTCT |
| 47. | 238-257 | TGGTTTCCTCATTCCGGCTC |
| 48. | 239-258 | CTGGTTTCCTCATTCCGGCT |
| 49. | 240-259 | CCTGGTTTCCTCATTCCGGC |
| 50. | 241-260 | CCCTGGTTTCCTCATTCCGG |
| 51. | 242-261 | GCCCTGGTTTCCTCATTCCG |
| 52. | 243-262 | GGCCCTGGTTTCCTCATTCC |
| 53. | 382-401 | CTTTCCTCTCATTGGCATCT |
| 54. | 383-402 | CCTTTCCTCTCATTGGCATC |
| 55. | 384-403 | ACCTTTCCTCTCATTGGCAT |
| 56. | 799-818 | CACGCTGGGTTGTGAGAATC |
| 57. | 800-819 | TCACGCTGGGTTGTGAGAAT |
| 58. | 801-820 | CTCACGCTGGGTTGTGAGAA |
| 59. | 802-821 | GCTCACGCTGGGTTGTGAGA |
| 60. | 806-825 | TTCGGCTCACGCTGGGTTGT |
| 61. | 898-917 | GATTCATGCCCTTCTCTTTG |
| 62 | 899-918 | CGATTCATGCCCTTCTCTTT |
| 63. | 900-919 | CCGATTCATGCCCTTCTCTT |
| 64. | 901-920 | CCCGATTCATGCCCTTCTCT |
| 65. | 902-921 | TCCCGATTCATGCCCTTCTC |
| 66. | 903-922 | ATCCCGATTCATGCCCTTCT |
| 67. | 904-923 | CATCCCGATTCATGCCCTTC |
| 68. | 905-924 | TCATCCCGATTCATGCCCTT |
| 69 | 906-925 | ATCATCCCGATTCATGCCCT |
| 70. | 907-926 | GATCATCCCGATTCATGCCC |
| 71. | 908-927 | CGATCATCCCGATTCATGCC |
| 72. | 917-936 | CCATGGATCCGATCATCCCG |
| 73. | 918-937 | TCCATGGATCCGATCATCCC |
| 74. | 1020-1039 | GTCGTGTACCAGCTGCTGCT |
| 75. | 1021-1040 | TGTCGTGTACCAGCTGCTGC |
| 76. | 1022-1041 | TTGTCGTGTACCAGCTGCTG |
| 77. | 1023-1042 | CTTGTCGTGTACCAGCTGCT |
| 78. | 1024-1043 | ACTTGTCGTGTACCAGCTGC |
| 79. | 1029-1048 | GCAGTACTTGTCGTGTACCA |
| 80. | 1030-1049 | TGCAGTACTTGTCGTGTACC |
| 81. | 1031-1050 | TTGCAGTACTTGTCGTGTAC |
| 82. | 1032-1051 | TTTGCAGTACTTGTCGTGTA |
| 83. | 1033-1052 | CTTTGCAGTACTTGTCGTGT |
| 84. | 1034-1053 | TCTTTGCAGTACTTGTCGTG |
| 85. | 1035-1054 | ATCTTTGCAGTACTTGTCGT |
| 86. | 1036-1055 | GATCTTTGCAGTACTTGTCG |
| 87. | 1058-1077 | GGTTTTGTTCCGAAGCCCAT |
| 88. | 1059-1078 | AGGTTTTGTTCCGAAGCCCA |
| 89. | 1060-1079 | GAGGTTTTGTTCCGAAGCCC |
| 90. | 1061-1080 | CGAGGTTTTGTTCCGAAGCC |
| 91. | 1062-1081 | ACGAGGTTTTGTTCCGAAGC |
| 92. | 1063-1082 | GACGAGGTTTTGTTCCGAAG |
| 93. | 1064-1083 | TGACGAGGTTTTGTTCCGAA |
| 94. | 1065-1084 | GTGACGAGGTTTTGTTCCGA |
| 95. | 1066-1085 | TGTGACGAGGTTTTGTTCCG |
| 96. | 1070-1089 | GTAATGTGACGAGGTTTTGT |
| 97. | 1071-1090 | GGTAATGTGACGAGGTTTTG |
| 98. | 1072-1091 | GGGTAATGTGACGAGGTTTT |
| 99. | 1073-1092 | GGGGTAATGTGACGAGGTTT |
| 100. | 1074-1093 | GGGGGTAATGTGACGAGGTT |
| 101. | 1075-1094 | AGGGGGTAATGTGACGAGGT |
| 102. | 1076-1095 | AAGGGGGTAATGTGACGAGG |
| 103. | 1077-1096 | GAAGGGGGTAATGTGACGAG |
| 104. | 1078-1097 | TGAAGGGGGTAATGTGACGA |
| 105. | 1171-1190 | AGGTCCCAAATCCCATGAGG |
| 106. | 1172-1191 | GAGGTCCCAAATCCCATGAG |
| 107. | 1173-1192 | GGAGGTCCCAAATCCCATGA |
| 108. | 1248-1267 | CACCTGATCAAATTTCTCCT |
| 109. | 1249-1268 | ACACCTGATCAAATTTCTCC |
| 110. | 1250-1269 | CACACCTGATCAAATTTCTC |
| 111. | 1251-1270 | GCACACCTGATCAAATTTCT |
| 112. | 1252-1271 | GGCACACCTGATCAAATTTC |
| 113. | 1253-1272 | TGGCACACCTGATCAAATTT |
| 114. | 1254-1273 | CTGGCACACCTGATCAAATT |
| 115. | 1255-1274 | ACTGGCACACCTGATCAAAT |
| 116. | 1256-1275 | CACTGGCACACCTGATCAAA |
| 117. | 1257-1276 | CCACTGGCACACCTGATCAA |
| 118. | 1258-1277 | CCCACTGGCACACCTGATCA |
| 119. | 1297-1316 | TTTGGATCAGCGAGTTCTTG |
| 120. | 1298-1317 | ATTTGGATCAGCGAGTTCTT |
| 121. | 1299-1318 | CATTTGGATCAGCGAGTTCT |
| 122. | 1393-1412 | TTAGGACATGGTTCATGGTA |
| 123. | 1394-1413 | CTTAGGACATGGTTCATGGT |
| 124. | 1395-1414 | GCTTAGGACATGGTTCATGG |
| 125. | 1396-1415 | AGCTTAGGACATGGTTCATG |
| 126. | 1397-1416 | CAGCTTAGGACATGGTTCAT |
| 127. | 1398-1417 | ACAGCTTAGGACATGGTTCA |
| 128. | 1399-1418 | CACAGCTTAGGACATGGTTC |
| 129. | 1400-1419 | ACACAGCTTAGGACATGGTT |
| 130. | 1401-1420 | GACACAGCTTAGGACATGGT |
| 131. | 1402-1421 | TGACACAGCTTAGGACATGG |
| 132. | 1403-1422 | TTGACACAGCTTAGGACATG |
| 133. | 1404-1423 | CTTGACACAGCTTAGGACAT |
| 134. | 1539-1558 | ATGGGCGAAGTCCTTTGGGG |
| 135. | 1540-1559 | TATGGGCGAAGTCCTTTGGG |
| 136. | 1541-1560 | TTATGGGCGAAGTCCTTTGG |
| 137. | 1637-1656 | TCCTTGATATCCTGCTGGAT |
| 138. | 1638-1657 | CTCCTTGATATCCTGCTGGA |
| 139. | 1639-1658 | GCTCCTTGATATCCTGCTGG |
| 140. | 1640-1659 | AGCTCCTTGATATCCTGCTG |
| 141. | 1641-1660 | CAGCTCCTTGATATCCTGCT |
| 142. | 1642-1661 | GCAGCTCCTTGATATCCTGC |
| 143. | 1643-1662 | AGCAGCTCCTTGATATCCTG |
| 144. | 1644-1663 | CAGCAGCTCCTTGATATCCT |
| 145. | 1645-1664 | CCAGCAGCTCCTTGATATCC |
| 146. | 1646-1665 | TCCAGCAGCTCCTTGATATC |
| 147. | 1647-1666 | CTCCAGCAGCTCCTTGATAT |
| 148. | 1648-1667 | GCTCCAGCAGCTCCTTGATA |
| 149. | 1649-1668 | GGCTCCAGCAGCTCCTTGAT |
| 150. | 1683-1702 | AGTGAGGGCAGGGCTTAGTC |
| 151. | 1684-1703 | CAGTGAGGGCAGGGCTTAGT |
| 152. | 1686-1705 | TGCAGTGAGGGCAGGGCTTA |
| 153. | 1892-1911 | GTTCGGAGGGCAAGAGTGAT |
| 154. | 1893-1912 | CGTTCGGAGGGCAAGAGTGA |
| 155. | 1895-1914 | AGCGTTCGGAGGGCAAGAGT |
| 156. | 1896-1915 | AAGCGTTCGGAGGGCAAGAG |
| 157. | 1930-1949 | GGGTCAGAGAGTGGCCTTCA |
| 158. | 1931-1950 | TGGGTCAGAGAGTGGCCTTC |
| 159. | 1932-1951 | TTGGGTCAGAGAGTGGCCTT |
| 160. | 1933-1952 | ATTGGGTCAGAGAGTGGCCT |
| 161. | 1934-1953 | AATTGGGTCAGAGAGTGGCC |
| 162. | 1935-1954 | AAATTGGGTCAGAGAGTGGC |
| 163. | 1936-1955 | CAAATTGGGTCAGAGAGTGG |
| 164. | 1937-1956 | ACAAATTGGGTCAGAGAGTG |
| 165. | 1938-1957 | AACAAATTGGGTCAGAGAGT |
| 166. | 1939-1958 | GAACAAATTGGGTCAGAGAG |
| 167. | 1972-1991 | GCTCACTGTTCAGGAAATGA |
| 168. | 1973-1992 | TGCTCACTGTTCAGGAAATG |
| 169. | 1974-1993 | GTGCTCACTGTTCAGGAAAT |
| 170. | 1975-1994 | TGTGCTCACTGTTCAGGAAA |
| 171. | 1976-1995 | TTGTGCTCACTGTTCAGGAA |
| 172. | 1977-1996 | CTTGTGCTCACTGTTCAGGA |
| 173. | 1978-1997 | CCTTGTGCTCACTGTTCAGG |
| 174. | 1979-1998 | TCCTTGTGCTCACTGTTCAG |
| 175. | 1980-1999 | CTCCTTGTGCTCACTGTTCA |
| 176. | 1981-2000 | TCTCCTTGTGCTCACTGTTC |
| 177. | 1982-2001 | ATCTCCTTGTGCTCACTGTT |
| 178. | 1983-2002 | GATCTCCTTGTGCTCACTGT |
| 179. | 2037-2056 | GAGGTGGATGGAGGGTGTGA |
| 180. | 2038-2057 | TGAGGTGGATGGAGGGTGTG |
| 181. | 2039-2058 | ATGAGGTGGATGGAGGGTGT |
| 182. | 2040-2059 | GATGAGGTGGATGGAGGGTG |
| 183. | 2041-2060 | TGATGAGGTGGATGGAGGGT |
| 184. | 2042-2061 | CTGATGAGGTGGATGGAGGG |
| 185. | 2043-2062 | ACTGATGAGGTGGATGGAGG |
| 186. | 2148-2167 | GTAGCGAATGTCAGGGTCAG |
| 187. | 2149-2168 | AGTAGCGAATGTCAGGGTCA |
| 188. | 2150-2169 | CAGTAGCGAATGTCAGGGTC |
| 189. | 2151-2170 | ACAGTAGCGAATGTCAGGGT |
| 190. | 2152-2171 | CACAGTAGCGAATGTCAGGG |
| 191. | 2296-2315 | GGTTCATGCTACTGAGTCGG |
| 192. | 2297-2316 | GGGTTCATGCTACTGAGTCG |
| 193. | 2298-2317 | AGGGTTCATGCTACTGAGTC |
| 194. | 2299-2318 | CAGGGTTCATGCTACTGAGT |
| 195. | 2300-2319 | GCAGGGTTCATGCTACTGAG |
| 196. | 2301-2320 | GGCAGGGTTCATGCTACTGA |
| 197. | 2302-2321 | AGGCAGGGTTCATGCTACTG |
| 198. | 2303-2322 | AAGGCAGGGTTCATGCTACT |
| 199. | 2304-2323 | AAAGGCAGGGTTCATGCTAC |
| 200. | 2305-2324 | CAAAGGCAGGGTTCATGCTA |
| 201. | 2306-2325 | ACAAAGGCAGGGTTCATGCT |
| 202. | 2307-2326 | GACAAAGGCAGGGTTCATGC |
| 203. | 2502-2521 | GTTTGGATCAGGGTCTGGAT |
| 204. | 2503-2522 | GGTTTGGATCAGGGTCTGGA |
| 205. | 2504-2523 | GGGTTTGGATCAGGGTCTGG |
| 206. | 2505-2524 | TGGGTTTGGATCAGGGTCTG |
| 207. | 2506-2525 | CTGGGTTTGGATCAGGGTCT |
| 208. | 2507-2526 | CCTGGGTTTGGATCAGGGTC |
| 209. | 2508-2527 | ACCTGGGTTTGGATCAGGGT |
| 210. | 2509-2528 | CACCTGGGTTTGGATCAGGG |
| 211. | 2510-2529 | ACACCTGGGTTTGGATCAGG |
| 212. | 2755-2774 | GCTCAGTCTTCAGAAAATTC |
| 213. | 2757-2776 | CTGCTCAGTCTTCAGAAAAT |
| 214. | 2758-2777 | TCTGCTCAGTCTTCAGAAAA |
| 215. | 2759-2778 | TTCTGCTCAGTCTTCAGAAA |
| 216. | 2760-2779 | GTTCTGCTCAGTCTTCAGAA |
| 217. | 2761-2780 | GGTTCTGCTCAGTCTTCAGA |
| 218. | 2762-2781 | TGGTTCTGCTCAGTCTTCAG |
| 219. | 2763-2782 | CTGGTTCTGCTCAGTCTTCA |
| 220. | 2764-2783 | CCTGGTTCTGCTCAGTCTTC |
| 221. | 2954-2973 | GGCAAGTTTCCCATGTTGAC |
| 222. | 2955-2974 | AGGCAAGTTTCCCATGTTGA |
| 223. | 2956-2975 | GAGGCAAGTTTCCCATGTTG |
| 224. | 2957-2976 | AGAGGCAAGTTTCCCATGTT |
| 225. | 2958-2977 | CAGAGGCAAGTTTCCCATGT |
| 226. | 3024-3043 | AGAGAGTGACTGGTCTCGGA |
| 227. | 3025-3044 | GAGAGAGTGACTGGTCTCGG |
| 228. | 3027-3046 | ATGAGAGAGTGACTGGTCTC |
| 229. | 3028-3047 | GATGAGAGAGTGACTGGTCT |
| 230. | 3029-3048 | TGATGAGAGAGTGACTGGTC |
| 231. | 3030-3049 | ATGATGAGAGAGTGACTGGT |
| 232. | 3031-3050 | GATGATGAGAGAGTGACTGG |
| 233. | 3032-3051 | TGATGATGAGAGAGTGACTG |
| 234. | 3033-3052 | GTGATGATGAGAGAGTGACT |
| 235. | 3034-3053 | TGTGATGATGAGAGAGTGAC |
| 236. | 3035-3054 | GTGTGATGATGAGAGAGTGA |
| 237. | 3036-3055 | GGTGTGATGATGAGAGAGTG |
| 238. | 3037-3056 | TGGTGTGATGATGAGAGAGT |
| 239. | 3038-3057 | ATGGTGTGATGATGAGAGAG |
| 240. | 3039-3058 | CATGGTGTGATGATGAGAGA |
| 241. | 3047-3066 | TGGACAACCATGGTGTGATG |
| 242. | 3058-3077 | AGGTGATGGCCTGGACAACC |
| 243. | 3059-3078 | AAGGTGATGGCCTGGACAAC |
| 244. | 3060-3079 | GAAGGTGATGGCCTGGACAA |
| 245. | 3061-3080 | TGAAGGTGATGGCCTGGACA |
| 246. | 3062-3081 | ATGAAGGTGATGGCCTGGAC |
| 247. | 3063-3082 | GATGAAGGTGATGGCCTGGA |
| 248. | 3064-3083 | AGATGAAGGTGATGGCCTGG |
| 249. | 3066-3085 | GAAGATGAAGGTGATGGCCT |
| 250. | 3067-3086 | TGAAGATGAAGGTGATGGCC |
| 251. | 3068-3087 | TTGAAGATGAAGGTGATGGC |
| 252. | 3069-3088 | CTTGAAGATGAAGGTGATGG |
| 253. | 3070-3089 | ACTTGAAGATGAAGGTGATG |
| 254. | 3071-3090 | GACTTGAAGATGAAGGTGAT |
| 255. | 3072-3091 | GGACTTGAAGATGAAGGTGA |
| 256. | 3073-3092 | GGGACTTGAAGATGAAGGTG |
| 257. | 3074-3093 | AGGGACTTGAAGATGAAGGT |
| 258. | 3075-3094 | CAGGGACTTGAAGATGAAGG |
| 259. | 3110-3129 | ATGACCTGGGGCAGGAACTG |
| 260. | 3111-3130 | CATGACCTGGGGCAGGAACT |
| 261. | 3134-3153 | CGAATGACGTTAAGGAACGT |
| 262. | 3135-3154 | TCGAATGACGTTAAGGAACG |
| 263. | 3136-3155 | CTCGAATGACGTTAAGGAAC |
| 264. | 3137-3156 | ACTCGAATGACGTTAAGGAA |
| 265. | 3138-3157 | GACTCGAATGACGTTAAGGA |
| 266. | 3139-3158 | AGACTCGAATGACGTTAAGG |
| 267. | 3364-3383 | GCATGTGTGGGATCAGCTGG |
| 268. | 3365-3384 | AGCATGTGTGGGATCAGCTG |
| 269. | 3366-3385 | CAGCATGTGTGGGATCAGCT |
| 270. | 3367-3386 | GCAGCATGTGTGGGATCAGC |
| 271. | 3368-3387 | CGCAGCATGTGTGGGATCAG |
| 272. | 3369-3388 | ACGCAGCATGTGTGGGATCA |
| 273. | 3370-3389 | CACGCAGCATGTGTGGGATC |
| 274. | 3371-3390 | ACACGCAGCATGTGTGGGAT |
| 275. | 3372-3391 | GACACGCAGCATGTGTGGGA |
| 276. | 3373-3392 | AGACACGCAGCATGTGTGGG |
| 277. | 3512-3531 | GGAGCTTCAGGGGCATCAAA |
| 278. | 3513-3532 | TGGAGCTTCAGGGGCATCAA |
| 279. | 3514-3533 | GTGGAGCTTCAGGGGCATCA |
| 280. | 3515-3534 | AGTGGAGCTTCAGGGGCATC |
| 281. | 3516-3535 | CAGTGGAGCTTCAGGGGCAT |
| 282. | 3520-3539 | ATGGCAGTGGAGCTTCAGGG |
| 283. | 3521-3540 | GATGGCAGTGGAGCTTCAGG |
| 284. | 3523-3542 | GAGATGGCAGTGGAGCTTCA |
| 285. | 3524-3543 | CGAGATGGCAGTGGAGCTTC |
| 286. | 3525-3544 | TCGAGATGGCAGTGGAGCTT |
| 287. | 3526-3545 | TTCGAGATGGCAGTGGAGCT |
| 288. | 3527-3546 | TTTCGAGATGGCAGTGGAGC |
| 289. | 3528-3547 | CTTTCGAGATGGCAGTGGAG |
| 290. | 3529-3548 | CCTTTCGAGATGGCAGTGGA |
| 291. | 3588-3607 | GGAGGCATAGTCAGTGAAAT |
| 292. | 3589-3608 | GGGAGGCATAGTCAGTGAAA |
| 293. | 3590-3609 | CGGGAGGCATAGTCAGTGAA |
| 294. | 3591-3610 | CCGGGAGGCATAGTCAGTGA |
| 295. | 3592-3611 | TCCGGGAGGCATAGTCAGTG |
| 296. | 3593-3612 | ATCCGGGAGGCATAGTCAGT |
| 297. | 3594-3613 | GATCCGGGAGGCATAGTCAG |
| 298. | 3603-3622 | AGGGTGAATGATCCGGGAGG |
| 299. | 3604-3623 | TAGGGTGAATGATCCGGGAG |
| 300. | 3605-3624 | ATAGGGTGAATGATCCGGGA |
| 301. | 3606-3625 | AATAGGGTGAATGATCCGGG |
| 302. | 4067-4086 | ATCGGGTTGTAGGCCTGTGC |
| 303. | 4068-4087 | CATCGGGTTGTAGGCCTGTG |
| 304. | 4069-4088 | CCATCGGGTTGTAGGCCTGT |
| 305. | 4121-4140 | TCATTCAGTTCAGACCAGCA |
| 306. | 4122-4141 | TTCATTCAGTTCAGACCAGC |
| 307. | 4123-4142 | CTTCATTCAGTTCAGACCAG |
| 308. | 4124-4143 | TCTTCATTCAGTTCAGACCA |
| 309. | 4125-4144 | ATCTTCATTCAGTTCAGACC |
| 310. | 4126-4145 | GATCTTCATTCAGTTCAGAC |
| 311. | 4190-4209 | GTGACTTCAGCGATGTCTTG |
| 312. | 4191-4210 | TGTGACTTCAGCGATGTCTT |
| 313. | 4192-4211 | GTGTGACTTCAGCGATGTCT |
| 314. | 4193-4212 | TGTGTGACTTCAGCGATGTC |
| 315. | 4194-4213 | CTGTGTGACTTCAGCGATGT |
| 316. | 4197-4216 | GGTCTGTGTGACTTCAGCGA |
| 317. | 4198-4217 | GGGTCTGTGTGACTTCAGCG |
| 318. | 4199-4218 | AGGGTCTGTGTGACTTCAGC |
| 319. | 4200-4219 | GAGGGTCTGTGTGACTTCAG |
| 320. | 4201-4220 | AGAGGGTCTGTGTGACTTCA |
| 321. | 4202-4221 | AAGAGGGTCTGTGTGACTTC |
| 322. | 4203-4222 | TAAGAGGGTCTGTGTGACTT |
| 323. | 4204-4223 | TTAAGAGGGTCTGTGTGACT |
| 324. | 4205-4224 | TTTAAGAGGGTCTGTGTGAC |
| 325. | 4206-4225 | GTTTAAGAGGGTCTGTGTGA |
| 326. | 4207-4226 | AGTTTAAGAGGGTCTGTGTG |
| 327. | 4208-4227 | AAGTTTAAGAGGGTCTGTGT |
| 328. | 4209-4228 | CAAGTTTAAGAGGGTCTGTG |
| 329. | 4210-4229 | CCAAGTTTAAGAGGGTCTGT |
| 330. | 4211-4230 | GCCAAGTTTAAGAGGGTCTG |
| 331. | 4212-4231 | AGCCAAGTTTAAGAGGGTCT |
| 332. | 4213-4232 | CAGCCAAGTTTAAGAGGGTC |
| 333. | 4214-4233 | TCAGCCAAGTTTAAGAGGGT |
| 334. | 4215-4234 | TTCAGCCAAGTTTAAGAGGG |
| 335. | 4322-4341 | TGTAGTGCTTTGGCATATGC |
| 336. | 4323-4342 | GTGTAGTGCTTTGGCATATG |
| 337. | 4324-4343 | AGTGTAGTGCTTTGGCATAT |
| 338. | 4325-4344 | TAGTGTAGTGCTTTGGCATA |
| 339. | 4326-4345 | GTAGTGTAGTGCTTTGGCAT |
| 340. | 4327-4346 | TGTAGTGTAGTGCTTTGGCA |
| 341. | 4328-4347 | TTGTAGTGTAGTGCTTTGGC |
| 342. | 4329-4348 | TTTGTAGTGTAGTGCTTTGG |
| 343. | 4330-4349 | CTTTGTAGTGTAGTGCTTTG |
| 344. | 4333-4352 | GTTCTTTGTAGTGTAGTGCT |
| 345. | 4334-4353 | AGTTCTTTGTAGTGTAGTGC |
| 346. | 4335-4354 | CAGTTCTTTGTAGTGTAGTG |
| 347. | 4336-4355 | CCAGTTCTTTGTAGTGTAGT |
| 348. | 4337-4356 | TCCAGTTCTTTGTAGTGTAG |
| 349. | 4338-4357 | CTCCAGTTCTTTGTAGTGTA |
| 350. | 4437-4456 | GGCATATTCTAACACTCCGG |
| 351. | 4438-4457 | TGGCATATTCTAACACTCCG |
| 352. | 4439-4458 | ATGGCATATTCTAACACTCC |
| 353. | 4440-4459 | CATGGCATATTCTAACACTC |
| 354. | 4453-4472 | CTCCAAAGTGTTTCATGGCA |
| 355. | 4454-4473 | TCTCCAAAGTGTTTCATGGC |
| 356. | 4490-4509 | TGCAGTTTCTCATACCAGGT |
| 357. | 4491-4510 | GTGCAGTTTCTCATACCAGG |
| 358. | 4492-4511 | CGTGCAGTTTCTCATACCAG |
| 359. | 4493-4512 | TCGTGCAGTTTCTCATACCA |
| 360. | 4494-4513 | CTCGTGCAGTTTCTCATACC |
| 361. | 4495-4514 | ACTCGTGCAGTTTCTCATAC |
| 362. | 4496-4515 | CACTCGTGCAGTTTCTCATA |
| 363. | 4497-4516 | CCACTCGTGCAGTTTCTCAT |
| 364. | 4498-4517 | CCCACTCGTGCAGTTTCTCA |
| 365. | 4499-4518 | TCCCACTCGTGCAGTTTCTC |
| 366. | 4500-4519 | CTCCCACTCGTGCAGTTTCT |
| 367. | 4501-4520 | CCTCCCACTCGTGCAGTTTC |
| 368. | 4502-4521 | TCCTCCCACTCGTGCAGTTT |
| 369. | 4503-4522 | ATCCTCCCACTCGTGCAGTT |
| 370. | 4504-4523 | CATCCTCCCACTCGTGCAGT |
| 371. | 4533-4552 | GTCCATTTTCTTGTCATAGG |
| 372. | 4536-4555 | GGTGTCCATTTTCTTGTCAT |
| 373. | 4537-4556 | TGGTGTCCATTTTCTTGTCA |
| 374. | 4538-4557 | TTGGTGTCCATTTTCTTGTC |
| 375. | 4539-4558 | GTTGGTGTCCATTTTCTTGT |
| 376. | 4540-4559 | TGTTGGTGTCCATTTTCTTG |
| 377. | 4542-4561 | CTTGTTGGTGTCCATTTTCT |
| 378. | 4543-4562 | CCTTGTTGGTGTCCATTTTC |
| 379. | 4652-4671 | GTCTCATCATTAACCAGGGT |
| 380. | 4653-4672 | GGTCTCATCATTAACCAGGG |
| 381. | 4654-4673 | GGGTCTCATCATTAACCAGG |
| 382. | 4655-4674 | TGGGTCTCATCATTAACCAG |
| 383. | 4656-4675 | TTGGGTCTCATCATTAACCA |
| 384. | 4657-4676 | CTTGGGTCTCATCATTAACC |
| 385. | 4658-4677 | GCTTGGGTCTCATCATTAAC |
| 386. | 4661-4680 | TTGGCTTGGGTCTCATCATT |
| 387. | 4662-4681 | CTTGGCTTGGGTCTCATCAT |
| 388. | 4663-4682 | TCTTGGCTTGGGTCTCATCA |
| 389. | 4947-4966 | TTTGTACTGGATAACCTCCT |
| 390. | 4948-4967 | GTTTGTACTGGATAACCTCC |
| 391. | 4949-4968 | AGTTTGTACTGGATAACCTC |
| 392. | 5192-5211 | GGATGGTCAAGTTGCCGAGA |
| 393. | 5193-5212 | AGGATGGTCAAGTTGCCGAG |
| 394. | 5194-5213 | GAGGATGGTCAAGTTGCCGA |
| 395. | 5195-5214 | AGAGGATGGTCAAGTTGCCG |
| 396. | 5196-5215 | CAGAGGATGGTCAAGTTGCC |
| 397. | 5197-5216 | GCAGAGGATGGTCAAGTTGC |
| 398. | 5198-5217 | GGCAGAGGATGGTCAAGTTG |
| 399. | 5199-5218 | TGGCAGAGGATGGTCAAGTT |
| 400. | 5200-5219 | TTGGCAGAGGATGGTCAAGT |
| 401. | 5201-5220 | GTTGGCAGAGGATGGTCAAG |
| 402. | 5202-5221 | TGTTGGCAGAGGATGGTCAA |
| 403. | 5203-5222 | CTGTTGGCAGAGGATGGTCA |
| 404. | 5204-5223 | ACTGTTGGCAGAGGATGGTC |
| 405. | 5205-5224 | AACTGTTGGCAGAGGATGGT |
| 406. | 5206-5225 | GAACTGTTGGCAGAGGATGG |
| 407. | 5207-5226 | TGAACTGTTGGCAGAGGATG |
| 408. | 5208-5227 | GTGAACTGTTGGCAGAGGAT |
| 409. | 5209-5228 | GGTGAACTGTTGGCAGAGGA |
| 410. | 5210-5229 | GGGTGAACTGTTGGCAGAGG |
| 411. | 5211-5230 | AGGGTGAACTGTTGGCAGAG |
| 412. | 5212-5231 | GAGGGTGAACTGTTGGCAGA |
| 413. | 5213-5232 | TGAGGGTGAACTGTTGGCAG |
| 414. | 5214-5233 | CTGAGGGTGAACTGTTGGCA |
| 415. | 5304-5323 | CTGCATGGTCTGGACAAAAT |
| 416. | 5305-5324 | GCTGCATGGTCTGGACAAAA |
| 417. | 5306-5325 | TGCTGCATGGTCTGGACAAA |
| 418. | 5307-5326 | TTGCTGCATGGTCTGGACAA |
| 419. | 5308-5327 | GTTGCTGCATGGTCTGGACA |
| 420. | 5309-5328 | TGTTGCTGCATGGTCTGGAC |
| 421. | 5310-5329 | CTGTTGCTGCATGGTCTGGA |
| 422. | 5437-5456 | GGATTGTGCTCTCATTGATG |
| 423. | 5438-5457 | GGGATTGTGCTCTCATTGAT |
| 424. | 5439-5458 | GGGGATTGTGCTCTCATTGA |
| 425. | 5440-5459 | TGGGGATTGTGCTCTCATTG |
| 426. | 5441-5460 | TTGGGGATTGTGCTCTCATT |
| 427. | 5442-5461 | TTTGGGGATTGTGCTCTCAT |
| 428. | 5443-5462 | CTTTGGGGATTGTGCTCTCA |
| 429. | 5444-5463 | ACTTTGGGGATTGTGCTCTC |
| 430. | 5445-5464 | CACTTTGGGGATTGTGCTCT |
| 431. | 5446-5465 | GCACTTTGGGGATTGTGCTC |
| 432. | 5447-5466 | AGCACTTTGGGGATTGTGCT |
| 433. | 5448-5467 | CAGCACTTTGGGGATTGTGC |
| 434. | 5449-5468 | GCAGCACTTTGGGGATTGTG |
| 435. | 5450-5469 | TGCAGCACTTTGGGGATTGT |
| 436. | 5451-5470 | CTGCAGCACTTTGGGGATTG |
| 437. | 5452-5471 | ACTGCAGCACTTTGGGGATT |
| 438. | 5542-5561 | TGTAGTGTAGCACAGCTTCG |
| 439. | 5543-5562 | TTGTAGTGTAGCACAGCTTC |
| 440. | 5544-5563 | TTTGTAGTGTAGCACAGCTT |
| 441. | 5545-5564 | GTTTGTAGTGTAGCACAGCT |
| 442. | 5546-5565 | TGTTTGTAGTGTAGCACAGC |
| 443. | 5547-5566 | ATGTTTGTAGTGTAGCACAG |
| 444. | 5548-5567 | GATGTTTGTAGTGTAGCACA |
| 445. | 5549-5568 | TGATGTTTGTAGTGTAGCAC |
| 446. | 5550-5569 | CTGATGTTTGTAGTGTAGCA |
| 447. | 5551-5570 | TCTGATGTTTGTAGTGTAGC |
| 448. | 5554-5573 | GGTTCTGATGTTTGTAGTGT |
| 449. | 5555-5574 | TGGTTCTGATGTTTGTAGTG |
| 450. | 5557-5576 | CTTGGTTCTGATGTTTGTAG |
| 451. | 5558-5577 | GCTTGGTTCTGATGTTTGTA |
| 452. | 5559-5578 | GGCTTGGTTCTGATGTTTGT |
| 453. | 5560-5579 | GGGCTTGGTTCTGATGTTTG |
| 454. | 5561-5580 | CGGGCTTGGTTCTGATGTTT |
| 455. | 5562-5581 | GCGGGCTTGGTTCTGATGTT |
| 456. | 5563-5582 | CGCGGGCTTGGTTCTGATGT |
| 457. | 5564-5583 | TCGCGGGCTTGGTTCTGATG |
| 458. | 5565-5584 | ATCGCGGGCTTGGTTCTGAT |
| 459. | 5566-5585 | CATCGCGGGCTTGGTTCTGA |
| 460. | 5567-5586 | TCATCGCGGGCTTGGTTCTG |
| 461. | 5568-5587 | CTCATCGCGGGCTTGGTTCT |
| 462. | 5569-5588 | TCTCATCGCGGGCTTGGTTC |
| 463. | 5570-5589 | TTCTCATCGCGGGCTTGGTT |
| 464. | 5571-5590 | CTTCTCATCGCGGGCTTGGT |
| 465. | 5837-5856 | GTATCCTGGAGGTTGTTGCC |
| 466. | 5838-5857 | TGTATCCTGGAGGTTGTTGC |
| 467. | 5839-5858 | GTGTATCCTGGAGGTTGTTG |
| 468. | 5840-5859 | AGTGTATCCTGGAGGTTGTT |
| 469. | 5841-5860 | GAGTGTATCCTGGAGGTTGT |
| 470. | 5962-5981 | TTGCAATGAGCTGAGGTATA |
| 471. | 6014-6033 | GTGAGAAGCTGGTGAATGAG |
| 472. | 6015-6034 | TGTGAGAAGCTGGTGAATGA |
| 473. | 6016-6035 | CTGTGAGAAGCTGGTGAATG |
| 474. | 6017-6036 | TCTGTGAGAAGCTGGTGAAT |
| 475. | 6018-6037 | GTCTGTGAGAAGCTGGTGAA |
| 476. | 6019-6038 | TGTCTGTGAGAAGCTGGTGA |
| 477. | 6020-6039 | ATGTCTGTGAGAAGCTGGTG |
| 478. | 6021-6040 | AATGTCTGTGAGAAGCTGGT |
| 479. | 6022-6041 | CAATGTCTGTGAGAAGCTGG |
| 480. | 6023-6042 | CCAATGTCTGTGAGAAGCTG |
| 481. | 6024-6043 | ACCAATGTCTGTGAGAAGCT |
| 482. | 6025-6044 | GACCAATGTCTGTGAGAAGC |
| 483. | 6026-6045 | CGACCAATGTCTGTGAGAAG |
| 484. | 6086-6105 | GCTGTCGTGGTAGACTTAGA |
| 485. | 6087-6106 | GGCTGTCGTGGTAGACTTAG |
| 486. | 6088-6107 | GGGCTGTCGTGGTAGACTTA |
| 487. | 6089-6108 | CGGGCTGTCGTGGTAGACTT |
| 488. | 6106-6125 | TGTTGGCTGCATTGTGCCGG |
| 489. | 6107-6126 | TTGTTGGCTGCATTGTGCCG |
| 490. | 6108-6127 | CTTGTTGGCTGCATTGTGCC |
| 491. | 6109-6128 | TCTTGTTGGCTGCATTGTGC |
| 492. | 6110-6129 | ATCTTGTTGGCTGCATTGTG |
| 493. | 6111-6130 | AATCTTGTTGGCTGCATTGT |
| 494. | 6112-6131 | GAATCTTGTTGGCTGCATTG |
| 495. | 6113-6132 | AGAATCTTGTTGGCTGCATT |
| 496. | 6114-6133 | CAGAATCTTGTTGGCTGCAT |
| 497. | 6135-6154 | GCTGTGCTCACACATGTTCT |
| 498. | 6136-6155 | TGCTGTGCTCACACATGTTC |
| 499. | 6137-6156 | TTGCTGTGCTCACACATGTT |
| 500. | 6138-6157 | GTTGCTGTGCTCACACATGT |
| 501. | 6139-6158 | TGTTGCTGTGCTCACACATG |
| 502. | 6140-6159 | GTGTTGCTGTGCTCACACAT |
| 503. | 6141-6160 | GGTGTTGCTGTGCTCACACA |
| 504. | 6142-6161 | GGGTGTTGCTGTGCTCACAC |
| 505. | 6143-6162 | AGGGTGTTGCTGTGCTCACA |
| 506. | 6144-6163 | CAGGGTGTTGCTGTGCTCAC |
| 507. | 6145-6164 | CCAGGGTGTTGCTGTGCTCA |
| 508. | 6146-6165 | ACCAGGGTGTTGCTGTGCTC |
| 509. | 6147-6166 | GACCAGGGTGTTGCTGTGCT |
| 510. | 6149-6168 | TGGACCAGGGTGTTGCTGTG |
| 511. | 6150-6169 | CTGGACCAGGGTGTTGCTGT |
| 512. | 6152-6171 | TGCTGGACCAGGGTGTTGCT |
| 513. | 6332-6351 | GTTTCCTTCAGAGTCTGGGG |
| 514. | 6333-6352 | TGTTTCCTTCAGAGTCTGGG |
| 515. | 6405-6424 | CCCTGATTTCATGTACTTCC |
| 516. | 6406-6425 | TCCCTGATTTCATGTACTTC |
| 517. | 6409-6428 | CATTCCCTGATTTCATGTAC |
| 518. | 6414-6433 | CTTGACATTCCCTGATTTCA |
| 519. | 6415-6434 | CCTTGACATTCCCTGATTTC |
| 520. | 6416-6435 | TCCTTGACATTCCCTGATTT |
| 521. | 6417-6436 | GTCCTTGACATTCCCTGATT |
| 522. | 6418-6437 | GGTCCTTGACATTCCCTGAT |
| 523. | 6419-6438 | AGGTCCTTGACATTCCCTGA |
| 524. | 6420-6439 | GAGGTCCTTGACATTCCCTG |
| 525. | 6421-6440 | TGAGGTCCTTGACATTCCCT |
| 526. | 6562-6581 | CATATGTTCCTGGCACAGCC |
| 527. | 6563-6582 | TCATATGTTCCTGGCACAGC |
| 528. | 6564-6583 | GTCATATGTTCCTGGCACAG |
| 529. | 6565-6584 | GGTCATATGTTCCTGGCACA |
| 530. | 6566-6585 | GGGTCATATGTTCCTGGCAC |
| 531. | 6567-6586 | GGGGTCATATGTTCCTGGCA |
| 532. | 6568-6587 | TGGGGTCATATGTTCCTGGC |
| 533. | 6569-6588 | TTGGGGTCATATGTTCCTGG |
| 534. | 6570-6589 | GTTGGGGTCATATGTTCCTG |
| 535. | 6571-6590 | GGTTGGGGTCATATGTTCCT |
| 536. | 6572-6591 | TGGTTGGGGTCATATGTTCC |
| 537. | 6573-6592 | CTGGTTGGGGTCATATGTTC |
| 538. | 6574-6593 | GCTGGTTGGGGTCATATGTT |
| 539. | 6575-6594 | GGCTGGTTGGGGTCATATGT |
| 540. | 6879-6898 | GATGAGGGCGTGCAGTGTGT |
| 541. | 6893-6912 | TCCCTGTAGTCCCGGATGAG |
| 542. | 6894-6913 | CTCCCTGTAGTCCCGGATGA |
| 543. | 6895-6914 | TCTCCCTGTAGTCCCGGATG |
| 544. | 6903-6922 | CTTCTTCTTCTCCCTGTAGT |
| 545. | 6904-6923 | TCTTCTTCTTCTCCCTGTAG |
| 546. | 6905-6924 | ATCTTCTTCTTCTCCCTGTA |
| 547. | 6906-6925 | GATCTTCTTCTTCTCCCTGT |
| 548. | 6907-6926 | GGATCTTCTTCTTCTCCCTG |
| 549. | 6908-6927 | AGGATCTTCTTCTTCTCCCT |
| 550. | 6915-6934 | GTTGAGAAGGATCTTCTTCT |
| 551. | 6916-6935 | TGTTGAGAAGGATCTTCTTC |
| 552. | 6918-6937 | GATGTTGAGAAGGATCTTCT |
| 553. | 6919-6938 | CGATGTTGAGAAGGATCTTC |
| 554. | 6920-6939 | TCGATGTTGAGAAGGATCTT |
| 555. | 6930-6949 | GATGCGATGCTCGATGTTGA |
| 556. | 6931-6950 | TGATGCGATGCTCGATGTTG |
| 557. | 6932-6951 | ATGATGCGATGCTCGATGTT |
| 558. | 6933-6952 | CATGATGCGATGCTCGATGT |
| 559. | 7158-7177 | GTTGGATGGGTGTCTATCTC |
| 560. | 7159-7178 | GGTTGGATGGGTGTCTATCT |
| 561. | 7160-7179 | AGGTTGGATGGGTGTCTATC |
| 562. | 7161-7180 | CAGGTTGGATGGGTGTCTAT |
| 563. | 7162-7181 | TCAGGTTGGATGGGTGTCTA |
| 564. | 7163-7182 | ATCAGGTTGGATGGGTGTCT |
| 565. | 7164-7183 | CATCAGGTTGGATGGGTGTC |
| 566. | 7165-7184 | GCATCAGGTTGGATGGGTGT |
| 567. | 7195-7214 | TGTGCAGGATCTTCCCACTC |
| 568. | 7196-7215 | ATGTGCAGGATCTTCCCACT |
| 569. | 7197-7216 | AATGTGCAGGATCTTCCCAC |
| 570. | 7198-7217 | CAATGTGCAGGATCTTCCCA |
| 571. | 7199-7218 | TCAATGTGCAGGATCTTCCC |
| 572. | 7200-7219 | GTCAATGTGCAGGATCTTCC |
| 573. | 7201-7220 | AGTCAATGTGCAGGATCTTC |
| 574. | 7202-7221 | AAGTCAATGTGCAGGATCTT |
| 575. | 7203-7222 | AAAGTCAATGTGCAGGATCT |
| 576. | 7204-7223 | CAAAGTCAATGTGCAGGATC |
| 577. | 7205-7224 | CCAAAGTCAATGTGCAGGAT |
| 578. | 7321-7340 | TTCTGTAGTTGCCATCCAGG |
| 579. | 7322-7341 | ATTCTGTAGTTGCCATCCAG |
| 580. | 7323-7342 | GATTCTGTAGTTGCCATCCA |
| 581. | 7324-7343 | TGATTCTGTAGTTGCCATCC |
| 582. | 7325-7344 | GTGATTCTGTAGTTGCCATC |
| 583. | 7326-7345 | TGTGATTCTGTAGTTGCCAT |
| 584. | 7327-7346 | ATGTGATTCTGTAGTTGCCA |
| 585. | 7328-7347 | CATGTGATTCTGTAGTTGCC |
| 586. | 7329-7348 | GCATGTGATTCTGTAGTTGC |
| 587. | 7330-7349 | GGCATGTGATTCTGTAGTTG |
| 588. | 7331-7350 | TGGCATGTGATTCTGTAGTT |
| 589. | 7332-7351 | GTGGCATGTGATTCTGTAGT |
| 590. | 7333-7352 | TGTGGCATGTGATTCTGTAG |
| 591. | 7334-7353 | GTGTGGCATGTGATTCTGTA |
| 592. | 7335-7354 | TGTGTGGCATGTGATTCTGT |
| 593. | 7336-7355 | CTGTGTGGCATGTGATTCTG |
| 594. | 7337-7356 | ACTGTGTGGCATGTGATTCT |
| 595. | 7445-7464 | TTGCCTTTGGTATTTGTGTC |
| 596. | 7446-7465 | GTTGCCTTTGGTATTTGTGT |
| 597. | 7447-7466 | TGTTGCCTTTGGTATTTGTG |
| 598. | 7449-7468 | CTTGTTGCCTTTGGTATTTG |
| 599. | 7450-7469 | GCTTGTTGCCTTTGGTATTT |
| 600. | 7451-7470 | CGCTTGTTGCCTTTGGTATT |
| 601. | 7452-7471 | TCGCTTGTTGCCTTTGGTAT |
| 602. | 7453-7472 | ATCGCTTGTTGCCTTTGGTA |
| 603. | 7454-7473 | GATCGCTTGTTGCCTTTGGT |
| 604. | 7461-7480 | CGTTCGGGATCGCTTGTTGC |
| 605. | 7462-7481 | TCGTTCGGGATCGCTTGTTG |
| 606. | 7463-7482 | CTCGTTCGGGATCGCTTGTT |
| 607. | 7464-7483 | CCTCGTTCGGGATCGCTTGT |
| 608. | 7465-7484 | TCCTCGTTCGGGATCGCTTG |
| 609. | 7466-7485 | GTCCTCGTTCGGGATCGCTT |
| 610. | 7476-7495 | GTAGGAATCCGTCCTCGTTC |
| 611. | 7477-7496 | AGTAGGAATCCGTCCTCGTT |
| 612. | 7478-7497 | GAGTAGGAATCCGTCCTCGT |
| 613. | 7727-7746 | GTCGCTTGTTTGATGAGCAG |
| 614. | 7728-7747 | TGTCGCTTGTTTGATGAGCA |
| 615. | 7855-7874 | GTTTCAGTTTAGTGGAAGCA |
| 616. | 7856-7875 | GGTTTCAGTTTAGTGGAAGC |
| 617. | 7857-7876 | TGGTTTCAGTTTAGTGGAAG |
| 618. | 7859-7878 | CATGGTTTCAGTTTAGTGGA |
| 619. | 7860-7879 | CCATGGTTTCAGTTTAGTGG |
| 620. | 8010-8029 | GTTTCACTGTCCTGGGAACC |
| 621. | 8011-8030 | TGTTTCACTGTCCTGGGAAC |
| 622. | 8012-8031 | ATGTTTCACTGTCCTGGGAA |
| 623. | 8013-8032 | TATGTTTCACTGTCCTGGGA |
| 624. | 8014-8033 | CTATGTTTCACTGTCCTGGG |
| 625. | 8015-8034 | ACTATGTTTCACTGTCCTGG |
| 626. | 8016-8035 | TACTATGTTTCACTGTCCTG |
| 627. | 8065-8084 | GTTATGGATCTTCTGTTCCC |
| 628. | 8066-8085 | AGTTATGGATCTTCTGTTCC |
| 629. | 8178-8197 | GCTGTGCTGAGTTTGCTGTA |
| 630. | 8179-8198 | GGCTGTGCTGAGTTTGCTGT |
| 631. | 8180-8199 | TGGCTGTGCTGAGTTTGCTG |
| 632. | 8181-8200 | TTGGCTGTGCTGAGTTTGCT |
| 633. | 8182-8201 | CTTGGCTGTGCTGAGTTTGC |
| 634. | 8183-8202 | TCTTGGCTGTGCTGAGTTTG |
| 635. | 8184-8203 | TTCTTGGCTGTGCTGAGTTT |
| 636. | 8213-8232 | CCTAATCCATGTTCTCCACG |
| 637. | 8214-8233 | TCCTAATCCATGTTCTCCAC |
| 638. | 8215-8234 | ATCCTAATCCATGTTCTCCA |
| 639. | 8216-8235 | GATCCTAATCCATGTTCTCC |
| 640. | 8217-8236 | GGATCCTAATCCATGTTCTC |
| 641. | 8218-8237 | AGGATCCTAATCCATGTTCT |
| 642. | 8246-8265 | GTCAGCATCTTCTGTGTCTT |
| 643. | 8247-8266 | GGTCAGCATCTTCTGTGTCT |
| 644. | 8333-8352 | CTTTTGTACTCATTTTGGCC |
| 645. | 8334-8353 | CCTTTTGTACTCATTTTGGC |
| 646. | 8382-8401 | AATTGAAGCGTGTGAGTCGC |
| 647. | 8383-8402 | GAATTGAAGCGTGTGAGTCG |
| 648. | 8384-8403 | TGAATTGAAGCGTGTGAGTC |
| 649. | 8385-8404 | TTGAATTGAAGCGTGTGAGT |
| 650. | 8388-8407 | GTCTTGAATTGAAGCGTGTG |
| 651. | 8389-8408 | GGTCTTGAATTGAAGCGTGT |
| 652. | 8390-8409 | AGGTCTTGAATTGAAGCGTG |
| 653. | 8391-8410 | CAGGTCTTGAATTGAAGCGT |
| 654. | 8634-8653 | GTTGAGTATTTGTTCTGCTC |
| 655. | 8678-8697 | ACATGGTGTCTAGACATGGC |
| 656. | 8716-8735 | CTGATGTCATTTATTGGCAC |

Any of the unmodified antisense oligonucleotides herein can have any number and order of nucleotides modified with phosphorothioate linkages. In some embodiments, all nucleotides can be modified with phosphorothioate linkages.

Additional examples of agents of this disclosure for inhibiting or suppressing expression of IRF5 include IRF5-specific phosphorothioate antisense oligonucleotides given in Table 3. Phosphorothioate linkages are designated by an asterisk (*).

TABLE 3
IRF5-specific phosphorothioate antisense oligonucleotides

| SEQ ID | | |
|---|---|---|
| NO: | Ref. | ANTISENSE SEQUENCE (5-3) |
| 657 | 1250 1269 | C*A*C*A*C*C*T*G*A*T*C*A*A*A*T*T*T*C*T*C |
| 658 | 1061 1080 | C*G*A*G*G*T*T*T*T*G*T*T*C*C*G*A*A*G*C*C |
| 659 | 1073 1092 | G*G*G*G*T*A*A*T*G*T*G*A*C*G*A*G*G*T*T*T |
| 660 | 96 115 | T*T*A*A*T*A*T*T*C*G*C*G*C*G*G*C*T*T*C*G |
| 661 | 102 121 | T*T*T*C*C*T*T*T*A*A*T*A*T*T*C*G*C*G*C*G |
| 662 | 103 122 | G*T*T*T*C*C*T*T*T*A*A*T*A*T*T*C*G*C*G*C |
| 663 | 806 825 | T*T*C*G*G*C*T*C*A*C*G*C*T*G*G*G*T*T*G*T |
| 664 | 1072 1091 | G*G*G*T*A*A*T*G*T*G*A*C*G*A*G*G*T*T*T*T |
| 665 | 1251 1270 | G*C*A*C*A*C*C*T*G*A*T*C*A*A*A*T*T*T*C*T |
| 666 | 2757 2776 | C*T*G*C*T*C*A*G*T*C*T*T*C*A*G*A*A*A*A*T |

In some embodiments, as discussed herein, the IRF5 antisense sequences can be gapmers formed by adding 1 to 5 protected ribo-nucleotides on each flank of the phosphorothioate deoxy-nucleotide sequences in Table 3. For example, the ribo-nucleotides can be protected with 2′-OMe, 2′-OEt, or 2′-O-MOE substituents, or with LNA, cMOE, or cEt bridges, as well as phosphorothioate linkages.

### Anti-Cancer Methods and Compositions

This invention includes agents for inhibiting or suppressing expression of IRF5 for treating or ameliorating the symptoms of cancer in a subject.

In further embodiments, this invention includes uses of a composition comprising an agent for inhibiting or suppressing expression of IRF5 in the preparation of a medicament for treating or ameliorating the symptoms of cancer in a subject.

In certain embodiments, this invention includes methods for treating or ameliorating the symptoms of cancer in a subject in need, the method comprising: preparing a pharmaceutical composition comprising an agent for inhibiting or suppressing expression of IRF5; and administering a therapeutically sufficient amount of the composition to the subject.

This invention includes agents for inhibiting or suppressing expression of IRF5 for treating or ameliorating the symptoms of cancer in a subject in combination with an agent for inhibiting or suppressing expression of TGF-β2.

In further embodiments, this invention includes uses of a composition comprising an agent for inhibiting or suppressing expression of IRF5 in combination with an agent for inhibiting or suppressing expression of TGF-β2 in the preparation of a medicament for treating or ameliorating the symptoms of cancer in a subject.

In certain embodiments, this invention includes methods for treating or ameliorating the symptoms of cancer in a subject in need, the method comprising: preparing a pharmaceutical composition comprising an agent for inhibiting or suppressing expression of IRF5 in combination with an agent for inhibiting or suppressing expression of TGF-β2; and administering a therapeutically sufficient amount of the composition to the subject.

This invention also includes agents for inhibiting or suppressing expression of IRF5 for treating or ameliorating the symptoms of cancer in a subject in combination with an agent for inhibiting or suppressing expression of TGF-β2 and an immune checkpoint inhibitor.

In further embodiments, this invention includes uses of a composition comprising an agent for inhibiting or suppressing expression of IRF5 in combination with an agent for inhibiting or suppressing expression of TGF-β2 and an immune checkpoint inhibitor in the preparation of a medicament for treating or ameliorating the symptoms of cancer in a subject.

In certain embodiments, this invention includes methods for treating or ameliorating the symptoms of cancer in a subject in need, the method comprising: preparing a pharmaceutical composition comprising an agent for inhibiting or suppressing expression of IRF5 in combination with an agent for inhibiting or suppressing expression of TGF-β2 and an immune checkpoint inhibitor; and administering a therapeutically sufficient amount of the composition to the subject.

This invention includes methods for treating or ameliorating the symptoms of cancer in a human or animal subject in need, by administering a therapeutically sufficient amount of a pharmaceutical composition comprising an agent for inhibiting or suppressing expression of TGF-β2 to the subject; and administering a therapeutically sufficient amount of a pharmaceutical composition comprising a checkpoint inhibitor to the subject.

In certain embodiments, this invention includes agents for inhibiting or suppressing expression of TGF-β2 in combination with an immune checkpoint inhibitor for use in treating or ameliorating the symptoms of cancer in a human subject or animal.

In additional aspects, this invention includes uses of a composition comprising an agent for inhibiting or suppressing expression of TGF-β2 in the preparation of a medicament for treating or ameliorating the symptoms of a cancer in a human subject or animal in combination with an immune checkpoint inhibitor.

Any of the foregoing therapies may be combined with an immune checkpoint inhibitor.

Any of the foregoing therapies may be combined with a standard of care treatment for cancer. Anti-cancer therapies and agents can be administered by infusion, injection, or intracranial continuous infusion.

Any of the foregoing therapies may be combined with one or more additional medicaments comprising a targeted cancer drug, a cancer growth blocker, or an EGFR inhibitor, erlotinib, gefitinib, afatinib, osimertinib, dacomitininb, and combinations thereof.

In some embodiments, any of the foregoing therapies may be combined with one or more additional medicaments which are targeted cancer drugs selected from bevacizumab, everolimus, belzutifan, dabrafenib, trametinib, and combinations thereof.

In further embodiments, any of the foregoing therapies may be combined with one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

In certain embodiments, any of the foregoing therapies may be combined with one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

### Human TGF-β2-Specific Phosphorothioate Antisense Oligodeoxynucleotide

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide (OT-101; AP 12009; Trabedersen), hereafter referred to as OT-101 or AP 12009, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 was designed to be complementary to a specific sequence of human TGF-β2 mRNA following expression of the gene.

OT-101 can be supplied as a lyophilized powder in 50 mL glass vials in three different quantities. Each vial is identified by the name of the investigational product, trial number, dosing group, mode of application, quantity of OT-101 contained (in mg), total volume after dissolving (in mL) and resulting concentration (in μM), name of sponsor, name of manufacturer, batch number, vial number, storage temperature, and expiry date. The study medication can be provided in closed units, packaged separately for each concentration. The packages may contain the appropriate vial(s) and all necessary components of the application system (i.e., syringes, tube, and filter). OT-101 lyophilized powder is dissolved in isotonic (0.9%) aqueous sodium chloride prior to use. A leaflet can be enclosed in the packaging with instructions on how to prepare the product for administration of the desired concentration.

Examples of agents of this disclosure for inhibiting or suppressing expression of TGF-β include antisense oligonucleotides specific for TGF-β1, TGF-β2, or TGF-β3.

Examples of agents of this disclosure for inhibiting or suppressing expression of TGF-β2 include TGF-β2-specific antisense oligonucleotides given in SEQ ID NOs: 667-802 in Table 4.

TABLE 4
TGF-β2-specific antisense oligonucleotides

| SEQ ID | | |
|---|---|---|
| NO: | Ref. | ANTISENSE SEQUENCE |
| 667 | 1676-1693 | CGGCATGTCT ATTTTGTA (OT-101) |
| 668 | — | GTTCGTTTAG AGAACAGATC |
| 669 | — | TAAAGTTCGT TTAGAGAACA G |
| 670 | — | AGCCCTGTAT ACGAC |
| 671 | — | GTAGGTAAAA ACCTAATAT |
| 672 | — | CGTTTAGAGA ACAGATCTAC |
| 673 | — | CATTGTAGAT GTCAAAAGCC |
| 674 | — | CTCCCTCATG GTGGCAGTTG A |
| 675 | 178-195 | TTTGTTCCTG GATGACTC |
| 676 | 179-196 | GTTTGTTCCT GGATGACT |
| 677 | 180-197 | AGTTTGTTCC TGGATGAC |
| 678 | 181-198 | CAGTTTGTTC CTGGATGA |
| 679 | 182-199 | TCAGTTTGTT CCTGGATG |
| 680 | 183-200 | CTCAGTTTGT TCCTGGAT |
| 681 | 716-733 | TGTGTGTGTG TGCGTGTG |
| 682 | 717-734 | GTGTGTGTGT GTGCGTGT |
| 683 | 718-735 | TGTGTGTGTG TGTGCGTG |
| 684 | 719-736 | GTGTGTGTGT GTGTGCGT |
| 685 | 720-737 | TGTGTGTGTG TGTGTGCG |
| 686 | 721-738 | GTGTGTGTGT GTGTGTGC |
| 687 | 722-739 | TGTGTGTGTG TGTGTGTG |
| 688 | 723-740 | GTGTGTGTGT GTGTGTGT |
| 689 | 724-741 | TGTGTGTGTG TGTGTGTG |
| 690 | 725-742 | GTGTGTGTGT GTGTGTGT |
| 691 | 726-743 | TGTGTGTGTG TGTGTGTG |
| 692 | 727-744 | GTGTGTGTGT GTGTGTGT |
| 693 | 728-745 | TGTGTGTGTG TGTGTGTG |
| 694 | 729-746 | GTGTGTGTGT GTGTGTGT |
| 695 | 730-747 | CGTGTGTGTG TGTGTGTG |
| 696 | 731-748 | GCGTGTGTGT GTGTGTGT |
| 697 | 732-749 | TGCGTGTGTG TGTGTGTG |
| 698 | 733-750 | GTGCGTGTGT GTGTGTGT |
| 699 | 971-988 | AGTGGCGGAT CTGAACTC |
| 700 | 972-989 | GAGTGGCGGA TCTGAACT |
| 701 | 1430-1447 | GAGTGTGCTG CAGGTAGA |
| 702 | 1432-1449 | TCGAGTGTGC TGCAGGTA |
| 703 | 1433-1450 | ATCGAGTGTG CTGCAGGT |
| 704 | 1570-1587 | GTGCTGTTGT AGATGGAA |
| 705 | 1571-1588 | GGTGCTGTTG TAGATGGA |
| 706 | 1572-1589 | TGGTGCTGTT GTAGATGG |
| 707 | 1573-1590 | CTGGTGCTGT TGTAGATG |
| 708 | 1574-1591 | CCTGGTGCTG TTGTAGAT |
| 709 | 1575-1592 | CCCTGGTGCT GTTGTAGA |
| 710 | 1576-1593 | TCCCTGGTGC TGTTGTAG |
| 711 | 1577-1594 | GTCCCTGGTG CTGTTGTA |
| 712 | 1578-1595 | AGTCCCTGGT GCTGTTGT |
| 713 | 1895-1912 | CTGGGTTGGA GATGTTAA |
| 714 | 1896-1913 | GCTGGGTTGG AGATGTTA |
| 715 | 1897-1914 | CGCTGGGTTG GAGATGTT |
| 716 | 1900-1917 | TAGCGCTGGG TTGGAGAT |
| 717 | 1903-1920 | ATGTAGCGCT GGGTTGGA |
| 718 | 1945-1962 | AGCCATTCGC CTTCTGCT |
| 719 | 1994-2011 | GTCTTTATGG TGAAGCCA |
| 720 | 1995-2012 | TGTCTTTATG GTGAAGCC |
| 721 | 1996-2013 | CTGTCTTTAT GGTGAAGC |
| 722 | 1997-2014 | CCTGTCTTTA TGGTGAAG |
| 723 | 2000-2017 | GTTCCTGTCT TTATGGTG |
| 724 | 2001-2018 | GGTTCCTGTC TTTATGGT |
| 725 | 2002-2019 | AGGTTCCTGT CTTTATGG |
| 726 | 2003-2020 | CAGGTTCCTG TCTTTATG |
| 727 | 2004-2021 | CCAGGTTCCT GTCTTTAT |
| 728 | 2183-2200 | GGTCTTCCCA CTGTTTTT |
| 729 | 2194-2211 | AGGAGATGTG GGGTCTTC |
| 730 | 2195-2212 | CAGGAGATGT GGGGTCTT |
| 731 | 2241-2258 | GGTTGGTCTG TTGTGACT |
| 732 | 2242-2259 | CGGTTGGTCT GTTGTGAC |
| 733 | 2243-2260 | CCGGTTGGTC TGTTGTGA |
| 734 | 2522-2539 | GAGAATGGTT AGAGGTTC |
| 735 | 2525-2542 | GTAGAGAATG GTTAGAGG |
| 736 | 2542-2559 | GGTGTTTTGC CAATGTAG |
| 737 | 2543-2560 | GGGTGTTTTG CCAATGTA |
| 738 | 2544-2561 | TGGGTGTTTT GCCAATGT |
| 739 | 2666-2683 | CATCATCGTT GTCGTCGT |
| 740 | 2979-2996 | GAACGGTACG TACAGCAA |
| 741 | 2980-2997 | GGAACGGTAC GTACAGCA |
| 742 | 2981-2998 | AGGAACGGTA CGTACAGC |
| 743 | 2982-2999 | TAGGAACGGT ACGTACAG |
| 744 | 2983-3000 | ATAGGAACGG TACGTACA |
| 745 | 2984-3001 | GATAGGAACG GTACGTAC |
| 746 | 2985-3002 | GGATAGGAAC GGTACGTA |
| 747 | 2986-3003 | GGGATAGGAA CGGTACGT |
| 748 | 3029-3046 | GGGTGCCTAT TGCATAGC |
| 749 | 3030-3047 | AGGGTGCCTA TTGCATAG |
| 750 | 3031-3048 | AAGGGTGCCT ATTGCATA |
| 751 | 3032-3049 | GAAGGGTGCC TATTGCAT |
| 752 | 3033-3050 | GGAAGGGTGC CTATTGCA |
| 753 | 3035-3052 | TGGGAAGGGT GCCTATTG |
| 754 | 3036-3053 | ATGGGAAGGG TGCCTATT |
| 755 | 3037-3054 | AATGGGAAGG GTGCCTAT |
| 756 | 3038-3055 | GAATGGGAAG GGTGCCTA |
| 757 | 3039-3056 | AGAATGGGAA GGGTGCCT |
| 758 | 3040-3057 | AAGAATGGGA AGGGTGCC |
| 759 | 3041-3058 | TAAGAATGGG AAGGGTGC |
| 760 | 3042-3059 | GTAAGAATGG GAAGGGTG |
| 761 | 3043-3060 | AGTAAGAATG GGAAGGGT |
| 762 | 3044-3061 | GAGTAAGAAT GGGAAGGG |
| 763 | 3259-3276 | CAGACTTTCT CGGTCATA |
| 764 | 3260-3277 | GCAGACTTTC TCGGTCAT |
| 765 | 3261-3278 | TGCAGACTTT CTCGGTCA |
| 766 | 3262-3279 | ATGCAGACTT TCTCGGTC |
| 767 | 3263-3280 | AATGCAGACT TTCTCGGT |
| 768 | 3264-3281 | TAATGCAGAC TTTCTCGG |
| 769 | 4281-4298 | GACCTGGACT TTTTTCCC |
| 770 | 4282-4299 | TGACCTGGAC TTTTTTCC |
| 771 | 4283-4300 | CTGACCTGGA CTTTTTTC |
| 772 | 4284-4301 | GCTGACCTGG ACTTTTTT |
| 773 | 4467-4484 | CTGCAATGAT GTGGCAAA |
| 774 | 4468-4485 | TCTGCAATGA TGTGGCAA |
| 775 | 4469-4486 | TTCTGCAATG ATGTGGCA |
| 776 | 4470-4487 | CTTCTGCAAT GATGTGGC |
| 777 | 5062-5079 | GCTGCCCACT TGCATACT |
| 778 | 5569-5586 | GTTGGCAGAA CATAGAAC |
| 779 | 5570-5587 | CGTTGGCAGA ACATAGAA |
| 780 | 5571-5588 | GCGTTGGCAG AACATAGA |
| 781 | 5616-5633 | ATGGGGCTAC AGGGGATA |
| 782 | 5617-5634 | TATGGGGCTA CAGGGGAT |
| 783 | 5618-5635 | TTATGGGGCT ACAGGGGA |
| 784 | 5620-5637 | AGTTATGGGG CTACAGGG |
| 785 | 5621-5638 | AAGTTATGGG GCTACAGG |
| 786 | 5622-5639 | CAAGTTATGG GGCTACAG |
| 787 | 5623-5640 | CCAAGTTATG GGGCTACA |
| 788 | 5624-5641 | TCCAAGTTAT GGGGCTAC |
| 789 | 5625-5642 | ATCCAAGTTA TGGGGCTA |
| 790 | 5626-5643 | TATCCAAGTT ATGGGGCT |
| 791 | 5627-5644 | CTATCCAAGT TATGGGGC |
| 792 | 5759-5776 | ATTGGAGGAA ATAGGGTG |
| 793 | 5783-5800 | GTCTTGTAGG TAGCAGCC |
| 794 | 5784-5801 | GGTCTTGTAG GTAGCAGC |
| 795 | 5785-5802 | TGGTCTTGTA GGTAGCAG |
| 796 | 5786-5803 | CTGGTCTTGT AGGTAGCA |
| 797 | 5787-5804 | TCTGGTCTTG TAGGTAGC |
| 798 | 5788-5805 | GTCTGGTCTT GTAGGTAG |
| 799 | 5789-5806 | AGTCTGGTCT TGTAGGTA |
| 800 | 5790-5807 | GAGTCTGGTC TTGTAGGT |
| 801 | 5791-5808 | GGAGTCTGGT CTTGTAGG |
| 802 | 5792-5809 | AGGAGTCTGG TCTTGTAG |

The sequences of Table 4 can be chemically-modified to provide active variants thereof, LNA variants thereof, as well as gapmer variants thereof, as known in the art. The sequences of Table 4 can be used in any combination as active agents, such as pooling combinations.

It is understood that additional antisense oligonucleotides of this disclosure can be constructed based on the TGF-β2 gene sequence.

In some embodiments, an agent of antisense sequences can be gapmers formed by adding 1 to 5 protected ribo-nucleotides on each flank of the phosphorothioate deoxy-nucleotide sequences in Table 4. For example, the ribo-nucleotides can be protected with 2′-OMe, 2′-OEt, or 2′-O-MOE substituents, or with LNA, cMOE, or cEt bridges, as well as phosphorothioate linkages.

In some embodiments, an agent of antisense sequences can be a n-M-n RNA (2′-OMe)*-DNA*-RNA (2′-OMe)* gapmer, where n is from 3-7 and M is from 6-12. In certain embodiments, the gapmer can be a 3-10-3 or 5-10-5 LNA*-DNA*-LNA* or cEt*-DNA*-cEt* gapmer (* designates phosphorothioate linkages).

Examples of agents of this disclosure for inhibiting or suppressing expression of TGF-β2 include TGF-β2-specific antisense oligonucleotides given in SEQ ID NOs: 803-810 in Table 5.

TABLE 5
TGF-β2-specific phosphorothioate antisense oligonucleotides

| SEQ ID | | |
|---|---|---|
| NO: | Ref. | ANTISENSE SEQUENCE |
| 803 | 1676-1693 | C*G*G*C*A*T*G*T*C*T*A*T*T*T*T*G*T*A (OT-101) |
| 804 | — | G*T*T*C*G*T*T*T*A*G*A*G*A*A*C*A*G*A*T*C |
| 805 | — | T*A*A*A*G*T*T*C*G*T*T*T*A*G*A*G*A*A*C*A*G |
| 806 | — | A*G*C*C*C*T*G*T*A*T*A*C*G*A*C |
| 807 | — | G*T*A*G*G*T*A*A*A*A*A*C*C*T*A*A*T*A*T |
| 808 | — | C*G*T*T*T*A*G*A*G*A*A*C*A*G*A*T*C*T*A*C |
| 809 | — | C*A*T*T*G*T*A*G*A*T*G*T*C*A*A*A*A*G*C*C |
| 810 | — | C*T*C*C*C*T*C*A*T*G*G*T*G*G*C*A*G*T*T*G*A |

Embodiments of this invention further include pharmaceutical compositions for inhibiting or suppressing expression of TGF-β, or for treating or ameliorating the symptoms of cancer in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-2-specific antisense oligonucleotides. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

### OT-101 Antisense Oligonucleotide Drug Product

The API trabedersen/OT-101 is a synthetic 18-mer S-ODN consisting of the bases adenine (A), thymine (T), guanine (G), and cytosine (C), with all 3′-5′ linkages modified to phosphorothioates. This sulfur modification makes the drug more resistant to degradation, resulting in an increased stability in vitro and in vivo. Its molecular structure (nucleotide sequence) was designed to be complementary to a specific sequence of human transforming growth factor-beta 2 (TGF-2) mRNA. This sequence was selected among related molecules for its superior chemical and structural properties, biological activity, and specificity to achieve the best antisense effects in vitro and in vivo.

The chemical structure, exemplary of the phosphorothioate moieties (C-A-G), and the physical characteristics of trabedersen are shown in Table 6.

TABLE 6
Chemical and Physical Characteristics of Trabedersen

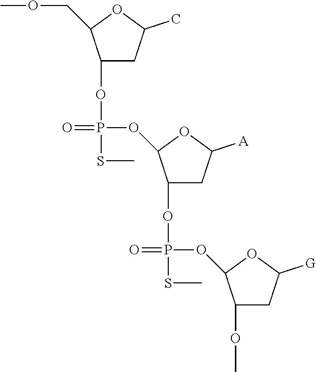

Phosphorothioate linkages
(C-A-G)

| Appearance: | White to off-white solid |
|---|---|
| Molecular Formula (sodium salt, | C₁₇₇N₂₀₈N₆₀O₉₄P₁₇S₁₇Na₁₇ |
| anhydrous): | |
| Molecular Weight (sodium salt, | 6143 g/mol |
| anhydrous): | |
| Number of moieties: | 18 |
| Backbone characteristics: | Phosphorothioates |
| Polymorphism: | No crystalline morphology in the solid |
| Water content: | Up to 15% |
| Solubility: | Very soluble in water (>50 mg/mL) |
| | Soluble in methanol (>8.5 mg/mL) |
| | Insoluble in iso-propanol (0.01 mg/mL) |
| pH of solution: | 6-8 |

The IMP is supplied as a sterile lyophilizate for solution for infusion in 50H glass vials (primary container) containing 7.37 mg trabedersen (intratumoral treatment) and in 20R glass vials (primary container) containing 250 mg trabedersen (intravenous treatment), respectively. No excipients are in the finished drug product. These glass vials are commonly used for parenterals. Sterile rubber stoppers appropriate for lyophilization seal the glass vial. The stopper is sealed with a crimping capsule that includes a colored flip-off cap. For clinical use, each vial is provided within a white-colored folding box to protect the vials from light exposure and damage during transport. Both, the glass vials and the folding boxes are labeled according to local requirements. The primary as well as secondary containers of the closure system fulfill international quality standards for the packaging of sterile solid drug products for injections.

### Checkpoint Inhibitor Agents

As referred to herein, checkpoint inhibitors as known in the art are immune checkpoint inhibitor agents. Checkpoint inhibitors are immunotherapy drugs which block checkpoint proteins from binding with their partner proteins. This prevents an “off” signal from being sent, which allows T cells to kill cancer cells. More particularly, checkpoint proteins, such as PD-1 on T cells, keep immune responses in check. Binding of PD-L1 to PD-1 keeps T cells from killing tumor cells. Thus, blocking the binding of PD-L1 to PD-1 with an immune checkpoint inhibitor may allow the T cells to kill tumor cells. The immune system is essentially turned back on so that T cells can attack cancer cells.

In some embodiments, a checkpoint inhibitor of this disclosure may be an inhibitor of CTLA-4, PD-1, or PD-L1.

In certain embodiments, a checkpoint inhibitor of this disclosure may be an inhibitor of PD-1.

In certain embodiments, a checkpoint inhibitor of this disclosure may be pembrolizumab.

In certain embodiments, a checkpoint inhibitor of this disclosure may be pembrolizumab, nivolumab, cemiplimab, spartalizumab, atezolizumab, avelumab, or durvalumab.

Without wishing to be bound by theory, the PD-1 receptor-ligand interaction can be a major pathway hijacked by tumors to suppress immune control. The normal function of PD-1, expressed on the cell surface of activated T cells under healthy conditions, is to down-modulate unwanted or excessive immune responses, including autoimmune reactions. Following T-cell stimulation, PD-1 recruits the tyrosine phosphatases, SHP-1 and SHP-2, to the immunoreceptor tyrosine-based switch motif within its cytoplasmic tail, leading to the dephosphorylation of effector molecules such as CD3 zeta (CD3ζ), protein kinase C-theta (PKCθ), and zeta-chain-associated protein kinase (ZAP70), which are involved in the CD3 T-cell signaling cascade.

Numbered embodiments of this invention include the following:

1) An agent for inhibiting or suppressing expression of IRF5 for treating or ameliorating the symptoms of cancer in a subject.

2) Use of a composition comprising an agent for inhibiting or suppressing expression of IRF5 in the preparation of a medicament for treating or ameliorating the symptoms of cancer in a subject.

3) A method for treating or ameliorating the symptoms of cancer in a subject in need, the method comprising:
preparing a pharmaceutical composition comprising an agent for inhibiting or suppressing expression of IRF5; and
administering a therapeutically sufficient amount of the composition to the subject.

4) The agent, use or method of any of embodiments 1-3, wherein the cancer is a glioma, low grade glioma, glioblastoma, diffuse intrinsic pontine glioma (DIPG), diffuse midline glioma (DMG), leptomeningeal or brain metastasis, brain or spinal cancer, or CNS tumors.

5) The agent, use or method of any of embodiments 1-4, wherein the cancer is a pancreatic cancer.

6) The agent, use or method of any of embodiments 1-5, comprising using one or more biomarkers to select the subjects who benefit from the agent, use or method, wherein the biomarkers are an elevated level of TGF-β2 and an elevated level of one or more of IFNGR2, JAK1, and STAT1.

7) The agent, use or method of any of embodiments 1-6, comprising using one or more biomarkers to select the subjects who benefit from the agent, use or method, wherein the biomarkers are levels of TGF-β2 and one or more of IRF5, TLR9, FOXP3, CCL22, CREB5, CD8a, CD86, CC14, CD163, ITGAX, and CD11c.

8) The agent, use or method of any of embodiments 1-7, wherein the agent, medicament or administration comprises one or more IRF5-specific antisense oligonucleotides complementary to a IRF5 transcript and 15-30 nucleotides in length.

9) The agent, use or method of any of embodiments 1-8, wherein the agent, medicament or administration comprises one or more IRF5-specific antisense oligonucleotides complementary to a IRF5 pre-RNA, pre-mRNA or mRNA and 18-21 nucleotides in length.

10) The agent, use or method of any of embodiments 1-9, wherein the agent, medicament or administration comprises one or more IRF5-specific antisense oligonucleotides complementary to a IRF5 transcript as in any of Table 1, Table 2, and Table 3.

11) The agent, use or method of any of embodiments 1-10, wherein the agent, medicament or administration comprises IRF5-specific antisense oligonucleotides SEQ ID NO:1 or SEQ ID NO:657.

12) The agent, use or method of any of embodiments 1-11, comprising a IRF5-specific antisense oligonucleotide as in any of Table 1, Table 2, and Table 3 having one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

13) The agent, use or method of any of embodiments 1-12, wherein the agent is conjugated to a polyethylene glycol, a lipid, or a triantenarry N-acteyl-galactosamine.

14) The agent, use or method of any of embodiments 1-13, comprising a carrier of sterile water for injection, saline, isotonic saline, phosphate buffered saline, or a combination thereof.

15) The agent, use or method of any of embodiments 1-14, wherein the agent, medicament or administration is substantially free of excipients.

16) The agent, use or method of any of embodiments 1-15, wherein the agent, medicament or administration is stable for at least 14 days in carrier at 37° C.

17) The agent, use or method of any of embodiments 1-16, wherein the agent, medicament or administration is combined with a standard of care treatment for the cancer.

18) The agent, use or method of any of embodiments 1-17, wherein the agent is administered by infusion, injection, or intracranial continuous infusion.

19) The agent, use or method of any of embodiments 1-18, comprising any one or more additional medicaments comprising a targeted cancer drug, a cancer growth blocker, or an EGFR inhibitor, erlotinib, gefitinib, afatinib, osimertinib, dacomitininb, and combinations thereof.

20) The agent, use or method of any of embodiments 1-19, comprising any one or more additional medicaments which are targeted cancer drugs selected from bevacizumab, everolimus, belzutifan, dabrafenib, trametinib, and combinations thereof.

21) The agent, use or method of any of embodiments 1-20, comprising any one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

22) The agent, use or method of any of embodiments 1-21, comprising any one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

23) The agent, use or method of any of embodiments 1-22, wherein the agent, medicament or administration decreases mortality rate of subjects at month 6, 12, 18, 24, 30, or 36.

24) The agent, use or method of any of embodiments 1-23, wherein the agent, medicament or administration increases survival rate of subjects at month 6, 12, 18, 24, 30, or 36.

25) A kit comprising:
an agent of any of embodiments 1-24; and
a carrier.

26) An agent for inhibiting or suppressing expression of IRF5 in combination with an agent for inhibiting or suppressing expression of TGF-β2 for treating or ameliorating the symptoms of cancer in a subject.

27) Use of a composition comprising an agent for inhibiting or suppressing expression of IRF5 in combination with an agent for inhibiting or suppressing expression of TGF-β2 in the preparation of a medicament for treating or ameliorating the symptoms of cancer in a subject.

28) A method for treating or ameliorating the symptoms of cancer in a subject in need, the method comprising:
preparing a pharmaceutical composition comprising an agent for inhibiting or suppressing expression of IRF5 in combination with an agent for inhibiting or suppressing expression of TGF-β2; and
administering a therapeutically sufficient amount of the composition to the subject.

29) The agent, use or method of any of embodiments 26-28, wherein the cancer is a glioma, low grade glioma, glioblastoma, diffuse intrinsic pontine glioma (DIPG), diffuse midline glioma (DMG), leptomeningeal or brain metastasis, brain or spinal cancer, or CNS tumors.

30) The agent, use or method of any of embodiments 26-29, wherein the cancer is a pancreatic cancer.

31) The agent, use or method of any of embodiments 26-30, comprising using one or more biomarkers to select subjects who benefit from the agent, use or method, wherein the biomarkers are levels of TGF-β2 and one or more of IFNGR2, STAT1, IRF1, IRF5, CD276, and CD204.

32) The agent, use or method of any of embodiments 26-31, wherein the cancer is low grade glioma having tumor cells exhibiting wild type IDH1 or IDH2 and one or more of upregulated IFNGR2, upregulated STAT1, upregulated IRF1, upregulated IRF5, upregulated CD276, and upregulated CD204.

33) The agent, use or method of any of embodiments 26-32, wherein the IRF5 agent, medicament or administration comprises one or more IRF5-specific antisense oligonucleotides complementary to a IRF5 transcript and 15-30 nucleotides in length.

34) The agent, use or method of any of embodiments 26-33, wherein the IRF5 agent, medicament or administration comprises one or more IRF5-specific antisense oligonucleotides complementary to a IRF5 pre-RNA, pre-mRNA or mRNA and 18-21 nucleotides in length.

35) The agent, use or method of any of embodiments 26-34, wherein the IRF5 agent, medicament or administration comprises one or more IRF5-specific antisense oligonucleotides complementary to a IRF5 transcript as in any of Table 1, Table 2, and Table 3.

36) The agent, use or method of any of embodiments 26-35, wherein the agent for inhibiting or suppressing expression of IRF5 is YE6144 (S,E)-N1-(6-Fluoro-3-(2-(6-morpholinopyridazin-3-yl) vinyl)-1H-indazol-5-yl) butane-1,2-diamine Hydrochloride, a cell-penetrating peptide inhibitor of IRF5 dimerization, an NLS peptide mimic, or a decoy peptide.

37) The agent, use or method of any of embodiments 26-36, wherein the TGF-β2 agent, medicament or administration comprises one or more TGF-β2-specific antisense oligonucleotides complementary to a TGF-β2 transcript and 15-30 nucleotides in length.

38) The agent, use or method of any of embodiments 26-37, wherein the TGF-β2 agent, medicament or administration comprises one or more TGF-β2-specific antisense oligonucleotides complementary to a TGF-β2 pre-RNA, pre-mRNA or mRNA and 18-21 nucleotides in length.

39) The agent, use or method of any of embodiments 26-38, wherein the TGF-β2 agent, medicament or administration comprises one or more TGF-β2-specific antisense oligonucleotides complementary to a TGF-β2 transcript as in any of Table 4 and Table 5.

40) The agent, use or method of any of embodiments 26-39, wherein the agent, medicament or administration comprises the combination SEQ ID NO:1 and SEQ ID NO:667, or SEQ ID NO:657 and SEQ ID NO:803.

41) The agent, use or method of any of embodiments 26-40, wherein the antisense oligonucleotides as in any of Table 4 and Table 5 comprise one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

42) The agent, use or method of any of embodiments 26-41, wherein the agent is conjugated to a polyethylene glycol, a lipid, or a triantenarry N-acteyl-galactosamine.

43) The agent, use or method of any of embodiments 26-42, comprising a carrier of sterile water for injection, saline, isotonic saline, phosphate buffered saline, or a combination thereof.

44) The agent, use or method of any of embodiments 26-43, wherein the agent, medicament or administration is substantially free of excipients.

45) The agent, use or method of any of embodiments 26-44, wherein the agent, medicament or administration is stable for at least 14 days in carrier at 37° C.

46) The agent, use or method of any of embodiments 26-45, wherein the agent, medicament or administration is combined with a standard of care treatment for the cancer.

47) The agent, use or method of any of embodiments 26-46, wherein the agent for inhibiting or suppressing expression of IRF5 and the agent for inhibiting or suppressing expression of TGF-β2 are administered concurrently, simultaneously, sequentially, or separately in time.

48) The agent, use or method of any of embodiments 26-47, wherein the agents are administered by infusion, injection, or intracranial continuous infusion.

49) The agent, use or method of any of embodiments 26-48, comprising any one or more additional medicaments comprising a targeted cancer drug, a cancer growth blocker, or an EGFR inhibitor, erlotinib, gefitinib, afatinib, osimertinib, dacomitininb, and combinations thereof.

50) The agent, use or method of any of embodiments 26-49, comprising any one or more additional medicaments which are targeted cancer drugs selected from bevacizumab, everolimus, belzutifan, dabrafenib, trametinib, and combinations thereof.

51) The agent, use or method of any of embodiments 26-50, comprising any one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

52) The agent, use or method of any of embodiments 26-51, comprising any one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

53) The agent, use or method of any of embodiments 26-52, wherein the agent, medicament or administration decreases mortality rate of subjects at month 6, 12, 18, 24, 30, or 36.

54) The agent, use or method of any of embodiments 26-53, wherein the agent, medicament or administration increases survival rate of subjects at month 6, 12, 18, 24, 30, or 36.

55) A kit comprising:
the agents of any of embodiments 26-54; and
a carrier.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The designations of agents, compounds and structures of this disclosure are meant to encompass all possible isomers, stereoisomers, diastereomers, enantiomers, and/or optical isomers that would be understood to exist for the specified structure, including any mixture, racemic or otherwise, thereof.

### EXAMPLES

Example 1: This example shows that clinical outcomes for pancreatic cancer patients benefitted from suppressing IRF5 in combination with suppressing TGF-β2.

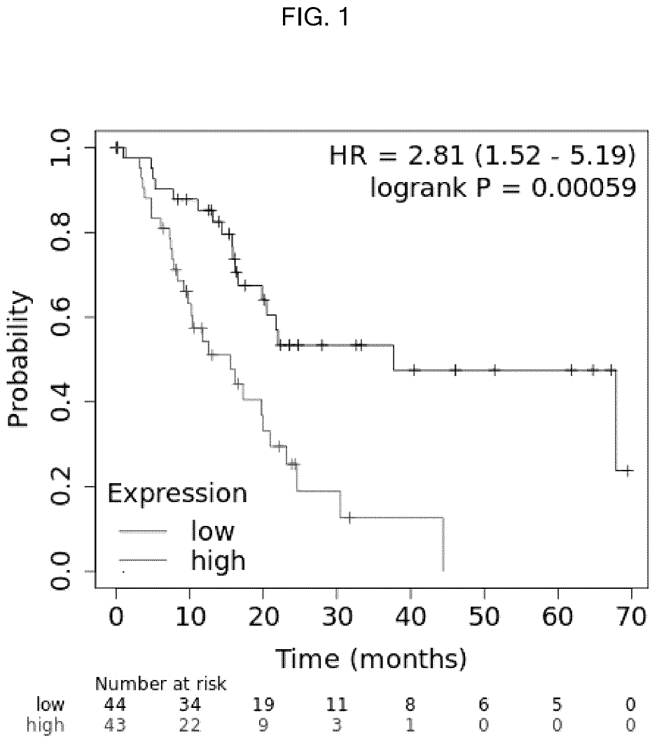

FIG. 1 shows results of a study of clinical outcomes for pancreatic cancer patients (PDAC) and the beneficial impact on overall survival for pancreatic cancer patients of the therapeutic use of an IRF5-specific antisense agent in combination with a TGF-β2-specific antisense agent. The Kaplan-Meier chart of FIG. 1 (KM Plotter) shows that overall survival was significantly improved for patients below median IRF5 and having low TGF-β2. This study established a basis for therapeutic use of an IRF5-specific antisense agent in combination with a TGF-β2-specific antisense agent for treating pancreatic cancer.

The median overall survival time for patients from the IRF5 (low)-TGFβ2 (low) group was 38 months (logrank P=0.00059), which was significantly and surprisingly increased over the 16 months observed for patients of the IRF5 (low)-TGFβ2 (high) group.

Example 2: This example shows that clinical outcomes for low grade glioma patients benefitted from suppressing IRF5.

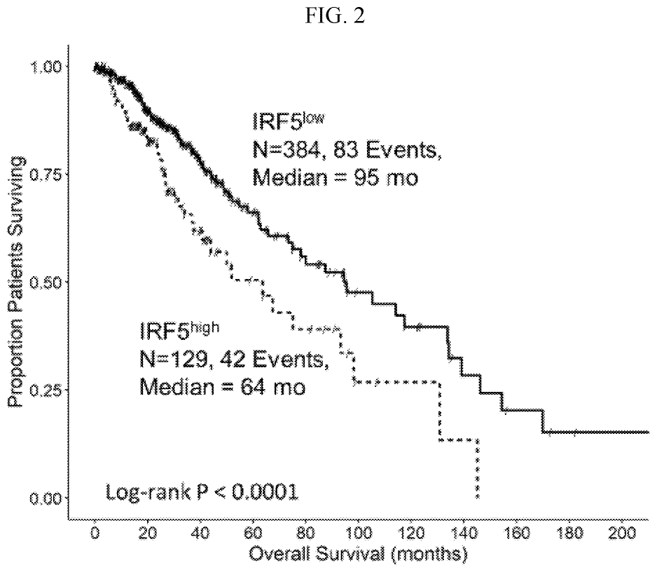

FIG. 2 shows results of a study of clinical outcomes for 513 low grade glioma patients (cBioPortal) and the beneficial impact on overall survival for low grade glioma patients of the therapeutic use of an IRF5-specific antisense agent. The Kaplan-Meier chart of FIG. 2 shows that overall survival was significantly improved for patients below median IRF5. This study established a basis for therapeutic use of an IRF5-specific antisense agent for treating low grade glioma.

The median overall survival time for patients from the IRF5 (low) group was 95 months (logrank P<0.0001), which was significantly and surprisingly increased over the 64 months observed for patients of the IRF5 (high) group.

Example 3: This example shows that clinical outcomes for low grade glioma patients benefitted from suppressing IRF5 in combination with suppressing TGF-β2.

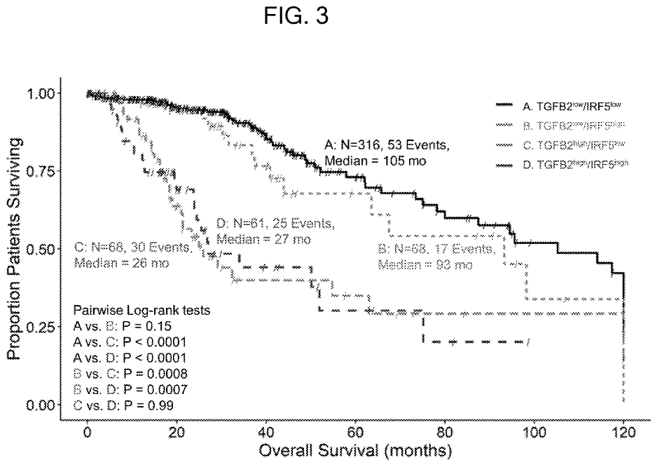

FIG. 3 shows results of a study of clinical outcomes for 513 low grade glioma patients (cBioPortal) and the beneficial impact on overall survival for low grade glioma patients of the therapeutic use of an IRF5-specific antisense agent in combination with a TGF-β2-specific antisense agent. The Kaplan-Meier chart of FIG. 3 shows that overall survival was significantly improved for patients below median IRF5 having low TGF-β2. This study established a basis for therapeutic use of an IRF5-specific antisense agent in combination with a TGF-β2-specific antisense agent for treating low grade glioma.

The median overall survival time for patients from the IRF5 (low)-TGFβ2 (low) group was 105 months (logrank P<0.0001), which was significantly and surprisingly increased over the 27 months observed for patients of the IRF5 (high)-TGFβ2 (high) group.

Example 4: This example shows that IFNGR2, JAKI, and STAT1 were biomarkers for selecting pediatric DIPG patients. The mRNA levels of IFNGR2, JAK1, and STAT1 can be used alone, or in any combination with each other or with other biomarkers and patient inclusion criterion to select pediatric DIPG patients.

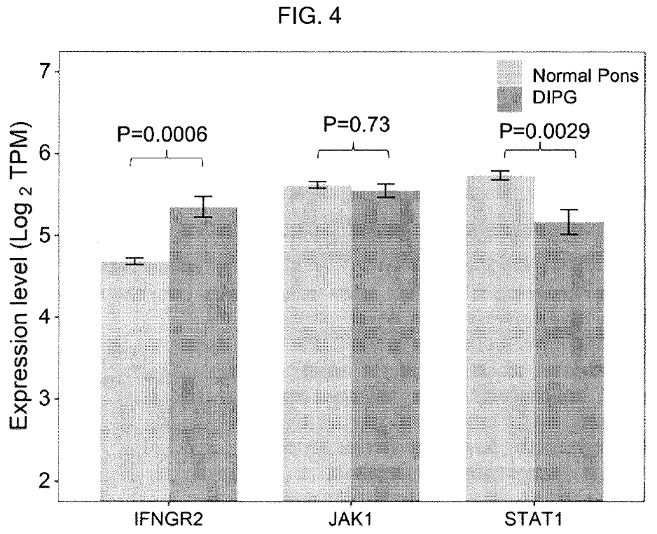

FIG. 4 shows results of a study of pediatric DIPG tumor samples. mRNA expression levels were obtained for IFNGR2 (N=45), JAK1 (N=45), and STAT1 (N=45), represented as log 2-transformed transcripts per million (TPM), for primary tumor samples. IFNGR2 mRNA levels were significantly upregulated in DIPG samples as compared to normal pons tissue (1.58-fold increase; P=0.0006). The pediatric DIPG patients whose brain tumors were localized to pons/brainstem included molecular subtype classifications DMG, H3K27M (N=23); DMG, H3K27M, TP53 (N=8); HGG, H3 wildtype (N=2); HGG, H3 wildtype, TP53 (N=1); HGG, to be classified (N=10) and 1 not determined. The mRNA expression levels in DIPG samples were compared to levels in normal pons samples from 29 pons regions (21 subjects). The bar charts illustrate mean expression levels for mRNA in tumor specimens (dark grey bars) as compared to normal pons samples (light grey bars). The statistical significance of differences in mRNA expression levels was assessed using two-way ANOVA.

Example 5: This example shows that CD14, CD163, and ITGAX were biomarkers for selecting pediatric DIPG patients. The mRNA levels of CD14, CD163, and ITGAX can be used alone, or in any combination with each other or with other biomarkers and patient inclusion criterion to select pediatric DIPG patients.

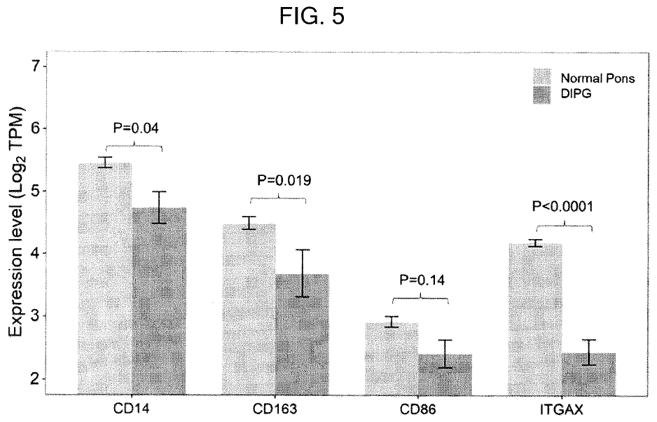

FIG. 5 shows results of a study of pediatric DIPG tumor samples. Antigen-presenting cell mRNA expression levels in pediatric DIPG tumors were downregulated as compared to normal brainstem tissue. CD14 (N=45), CD163 (N=45), CD86 (N=45), and ITGAX (N=45) mRNA expression levels were obtained (log 2 TPM) in pediatric DIPG samples and compared to the expression in normal pons samples. The bar charts illustrate mean expression levels for mRNA in tumor specimens from pediatric DIPG patients (dark grey bars) compared to normal pons samples (light grey bars). Compared to normal brainstem/pons tissue, CD14, CD163, and ITGAX mRNA expression in pediatric DIPG patients was significantly decreased by 1.64-fold (P=0.037), 1.75-fold (P=0.019), and 3.33-fold (P<0.0001), respectively. Differences in mRNA expression levels were assessed using two-way ANOVA.

1. An agent for inhibiting or suppressing expression of IRF5 for treating or ameliorating the symptoms of cancer in a subject.

2. (canceled)

3. (canceled)

4. The agent of claim 1, wherein the cancer is a glioma, low grade glioma, glioblastoma, diffuse intrinsic pontine glioma (DIPG), diffuse midline glioma (DMG), leptomeningeal or brain metastasis, brain or spinal cancer, or CNS tumors.

5. The agent of claim 1, wherein the cancer is a pancreatic cancer.

6. The agent of claim 1, comprising using one or more biomarkers to select the subjects, use or method, wherein the biomarkers are an elevated level of TGF-β2 and an elevated level of one or more of IFNGR2, JAK1, and STAT1.

7. The agent of claim 1, comprising using one or more biomarkers to select the subjects, wherein the biomarkers are levels of TGF-β2 and one or more of IRF5, TLR9, FOXP3, CCL22, CREB5, CD8a, CD86, CC14, CD163, ITGAX, and CD11c.

8. The agent of claim 1, wherein the agent comprises one or more IRF5-specific antisense oligonucleotides complementary to a IRF5 transcript and 15-30 nucleotides in length.

9. The agent of claim 1, wherein the agent comprises one or more IRF5-specific antisense oligonucleotides complementary to a IRF5 pre-RNA, pre-mRNA or mRNA and 18-21 nucleotides in length.

10. The agent of claim 1, wherein the agent comprises one or more IRF5-specific antisense oligonucleotides complementary to a IRF5 transcript as in any of Table 1, Table 2, and Table 3.

11. The agent of claim 1, wherein the agent comprises IRF5-specific antisense oligonucleotides CACACCTGATCAAATTTCTC SEQ ID NO: 1 or C*A*C*A*C*C*T*G*A*T*C*A*A*A*T*T*T*C*T*C SEQ ID NO:657.

12. The agent of claim 1, wherein the agent comprises a IRF5-specific antisense oligonucleotide as in any of Table 1, Table 2, and Table 3 having one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

13. The agent of claim 1, wherein the agent is conjugated to a polyethylene glycol, a lipid, or a triantenarry N-acteyl-galactosamine.

14. The agent of claim 1, comprising a carrier of sterile water for injection, saline, isotonic saline, phosphate buffered saline, or a combination thereof.

15. The agent of claim 1, wherein the agent is substantially free of excipients.

16. The agent of claim 1, wherein the agent is stable for at least 14 days in carrier at 37° C.

17. The agent of claim 1, wherein the agent is combined with a standard of care treatment for the cancer.

18. The agent of claim 1, wherein the agent is administered by infusion, injection, or intracranial continuous infusion.

19. The agent of claim 1, comprising any one or more additional medicaments comprising a targeted cancer drug, a cancer growth blocker, or an EGFR inhibitor, erlotinib, gefitinib, afatinib, osimertinib, dacomitininb, and combinations thereof.

20. The agent of claim 1, comprising any one or more additional medicaments which are targeted cancer drugs selected from bevacizumab, everolimus, belzutifan, dabrafenib, trametinib, and combinations thereof.

21. The agent of claim 1, comprising any one or more additional medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

22. The agent of claim 1, comprising any one or more additional medicaments for treatment of glioma selected from TMZ, radiation, and bevacizumab, or comprising any one or more additional medicaments for treatment of pancreatic cancer selected from paclitaxel, gemcitabine, 5FU, leucovrin, nal-Irinotecan, FOLFOX, FOLFIRI, FOLFIRINOX, and nal-FIRINOX.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)
